(19) United States
(12) Patent Application Publication
(10) Pub. No.: US 2025/0092097 A1
(43) Pub. Date: Mar. 20, 2025

(54) ANTIMICROBIAL TYPE-II BACTERIOCINS

(71) Applicant: Lundquist Institute for Biomedical Innovation at Harbor-UCLA Medical Center, Torrance, CA (US)

(72) Inventors: Nannette Y. Yount, San Juan Capistrano, CA (US); Michael R. Yeaman, Redondo Beach, CA (US)

(21) Appl. No.: 18/734,981

(22) Filed: Jun. 5, 2024

Related U.S. Application Data
(63) Continuation of application No. 17/468,311, filed on Sep. 7, 2021, now Pat. No. 12,037,368.
Application No. 17/468,311 is a continuation of application No. 16/612,245, filed on Nov. 8, 2019, now Pat. No. 11,124,547.
(60) Provisional application No. 62/505,015, filed on May 11, 2017.

(51) Int. Cl. C07K 14/32; A61K 38/16; A61K 45/06; C07K 14/195; C07K 14/325; G16B 30/10

(52) U.S. Cl. CPC C07K 14/32; A61K 38/164; A61K 45/06; C07K 14/195; C07K 14/325; G16B 30/10

(57) ABSTRACT

Computational systems and methods are described for identifying new type-II bacteriocins using a systemic consensus formula and other related criteria. Newly identified type-II bacteriocin peptides are tested experimentally and show potent microbiocidal activities.

### CROSS REFERENCE TO RELATED APPLICATIONS

This application is a continuation of U.S. application Ser. No. 17/468,311, filed Sep. 7, 2021, which is a continuation of U.S. application Ser. No. 16/612,245, filed Nov. 8, 2019, now U.S. Pat. No. 11,124,547, which is the U.S. national stage application of International Application No. PCT/US2018/032136, filed May 10, 2018, which claims the benefit under 35 U.S.C. § 119(e) of U.S. Provisional Application No. 62/505,015, filed May 11, 2017, the contents of which are incorporated by reference in their entirety into the present disclosure.

### SEQUENCE LISTING

The contents of the electronic sequence listing (254303.xml; Size: 1,706,125 bytes; and Date of Creation: Jun. 5, 2024) is herein incorporated by reference in its entirety.

### BACKGROUND

One of the most ancient arms of organismal host defense first arose in prokaryotes as a means to neutralize their competitors. This host defense armamentarium is likely more vast than the known microbial kingdom and is comprised of both ribosomally and non-ribosomally synthesized compounds. Of the ribosomally-synthesized compounds one of the most well characterized groups are the Class II bacteriocins which are synthesized primarily by Gram-positive organisms.

The majority of Class II bacteriocins are initially synthesized as precursors containing a relatively well conserved signal peptide domain. One characteristic feature of this domain, is the presence of a double-glycine motif which occurs just upstream of the signal peptide cleavage site. Processing of the signal peptide has been shown to be carried out by a class of dedicated membrane-bound ABC transporters which are conserved across many bacteriocin-expressing bacterial species.

### SUMMARY

It is contemplated that since Class II bacteriocins are processed via a common enzymatic process, most bacteriocin signal peptides contain elements that are conserved with a high degree of fidelity across many family members. As shown in the examples, an analysis of these conserved elements led to the creation of a consensus formula that was representative of a majority of the known class II bacteriocin families. This consensus formula was used a primary screen for previously uncharacterized class II bacteriocin families in online protein databases.

In addition to this conserved consensus, many class II bacteriocins were predicted, and/or shown, to form amphipathic helices when interacting with hydrophobic environments that mimic the microbial plasma membrane. Such amphipathic helices are common to many classes of antimicrobial peptides and are important for their membrane permeabilizing activities. With this discovery, as a means to selectively enrich for antimicrobial compounds, a new amphipathic pattern-search component is disclosed as a component of the search method.

This multi-component search method of the present technology recovered a large number of the known class II bacteriocins from a broad spectrum of organisms. Moreover, the method identified many putative bacteriocin sequences, some from organisms for which no bacteriocin peptides have been identified to date. A number of these putative bacteriocins were synthesized and found to have potent microbicidal activity against a broad spectrum of prototypic Gram-positive, Gram-negative and fungal organisms.

One embodiment of the present disclosure provides a peptide comprising an amino acid sequence selected from the group consisting of FKVIVTDAGHYPREWGKQLGKWIGSKIK (SEQ ID NO: 5), KRNYSIEKYVKNYIDFIKKAIDIFRPMPI (SEQ ID NO: 6), KTIATNATYYPNKWAKSAGKWIASKIK (SEQ ID NO: 7), QYDKTGYKIGKTVGTIVRKGFEIWSIFK (SEQ ID NO: 8), and an amino acid derived from SEQ ID NO: 5, 6, 7 or 8 with one amino acid substitution, wherein the peptide is not longer than 45 amino acid residues in length.

In some embodiments, the peptide has antimicrobial activity. In some embodiments, the peptide comprises the amino acid sequence of SEQ ID NO: 5, 6, 7, or 8.

In some embodiments, the peptide is not longer than 40 amino acid residues in length. In some embodiments, the peptide is not longer than 35 amino acid residues in length.

Also provided, in one embodiment, is a peptide comprising an amino acid sequence of Table 2 or 3 or SEQ ID NO: 328-1884 or an amino acid derived from a sequence of Table 2 or 3 or SEQ ID NO:328-1884 with one amino acid substitution, wherein the peptide is not longer than 45 amino acid residues in length.

In some embodiments, the peptide includes one or more non-natural amino acid residue.

Also provided, in one embodiment, is a composition comprising the peptide and a pharmaceutically acceptable carrier. In some embodiments, the composition further comprises an antimicrobial agent. In some embodiments, the antimicrobial agent is selected from the group consisting of imipenem, ceftazidime, colistin, chloroquine, artemisinin, vancomycin and daptomycin.

Also provided, in one embodiment, is a method of treating an infection in a patient in need thereof, comprising administering to the patient an effective amount of a peptide comprising an amino acid sequence of Table 2 or 3 or SEQ ID NO:328-1884 or an amino acid derived from a sequence of Table 2 or 3 or SEQ ID NO:328-1884 with one amino acid substitution. In some embodiments, the infection is caused by a Gram-negative bacterium, a Gram-positive bacterium or a fungus.

Yet another embodiment provides a computer-implemented method of identifying a type-II bacteriocin, comprising: searching in a protein database, with one or more processors, for proteins comprising a fragment matching a first consensus formula for type-II bacteriocin; filtering the searched proteins to remove proteins that do not contain an alpha-helical domain; calculating a score for the fragment in each of the searched proteins for one or more parameters selected from hydrophobic moment, mean hydrophobicity, net charge, frequencies or ratio of K and R, or isoelectric point; and identifying a type-II bacteriocin based on the scores.

In some embodiments, the first consensus formula comprises [LI]-[KREDNQSTYH]-X-[KREDNQSTYH]-X-[MLV]-X-X-[IVLT]-X-G-G, wherein X denotes any amino acid.

In some embodiments, the method further comprises searching in the protein database for proteins that match a hidden Markov Model built for type-II bacteriocin.

In some embodiments, the alpha-helical domain is identified with a second consensus formula comprising X-[VILMCFWYAG]-[KRHEDNQSTAG]-[KRHEDNQSTAG]-[VILMCFWYAG]-[VILMCFWYAG]-[KRHEDNQSTAG]-[KRHEDNQSTAG]-[VILMCFWYAG]-X-[KRHEDNQSTAG]-[VILMCFWYAG], wherein X denotes any amino acid residue.

In some embodiments, the parameters comprise hydrophobic moment and isoelectric point. In some embodiments, the search results are limited to proteins that are 80 amino acid residues or fewer in length and containing the fragment within the first 25 amino acid residues.

### BRIEF DESCRIPTION OF THE DRAWINGS

Certain features of various embodiments of the present technology are set forth with particularity in the appended claims. A better understanding of the features and advantages of the technology will be obtained by reference to the following detailed description that sets forth illustrative embodiments, in which the principles of the technology are utilized, and the accompanying drawings of which:

FIG. 1 illustrates an example process flow chart of a method, according to some implementations.

FIG. 2 shows the genomic environment surrounding putative bacteriocins. Analysis of 20 kb region surrounding putative bacteriocin genes. Red—putative bacteriocin; gray—hypothetical proteins; dark blue—C39 bacteriocin processing peptidase; medium blue—exinuclease ABC subunit; light blue—ABC transporter, ATP binding protein; green other enzyme; purple—polymerase related protein.

FIG. 3 shows positional and spatial amphipathic residue frequency. Percentages of individual residues on either the polar or non-polar peptide face of study peptides are represented as various color blocks. Residues above the x-axis are found on the polar face of retrieved peptides and residues below the axis are found on the non-polar face.

FIG. 4, with panels A-D, shows helical wheel analysis and biophysical properties of putative bacteriocins. Putative bacteriocins synthesized for assessment of antimicrobial activity. Arrows hydrophobic moment and direction. A. Peptide 1: A0RKV8 (+4.5), PI-10.7; Bacillus thuringiensis (G+); FKVIVTDAGHYPREWGKQLGKWIGSKIK, SEQ ID NO: 5 (28). B. Peptide 2: D6E338 (+4), PI 10.3; Eubacterium rectale; KRNYSIEKYVKNYIDFIKKAIDIFRPMPI, SEQ ID NO: 6 (29). C. Peptide 3: B3ZXE9 (+6), PI-10.9; Bacillus cereus; KTIATNATYYPNKWAKSAGKWIASKIK, SEQ ID NO: 7 (27). D. Peptide 4: R2S6C2 (+4), PI-10.5; Enterococcus pallens, QYDKTGYKIGKTVGTIVRKGFEIWSIFK, SEQ ID NO: 8 (28).

FIG. 5, with panels A and B, shows the antimicrobial activity of putative bacteriocins. Microbicidal activity of study test peptides versus a panel of prototypic gram-positive (S. aureus), gram-negative (S. typhimurium, P. aeruginosa, A. baumannii) and fungal (C. albicans) pathogens at two pH's representing: A—native physiologic (pH 7.5); or B—phagolysosomal (pH 5.5) environments.

FIG. 6 illustrates a block diagram of an example computer system in which any of the implementations described herein may be implemented.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “a peptide” includes a plurality of peptides.

### 1. Definitions

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

As used herein, the term “sequence identity” refers to a level of amino acid residue or nucleotide identity between two peptides or between two nucleic acid molecules. When a position in the compared sequence is occupied by the same base or amino acid, then the molecules are identical at that position. A peptide (or a polypeptide or peptide region) has a certain percentage (for example, at least about 60%, or at least about 65%, or at least about 70%, or at least about 75%, or at least about 80%, or at least about 83%, or at least about 85%, or at least about 90%, or at least about 95%, or at least about 98% or at least about 99%) of “sequence identity” to another sequence means that, when aligned, that percentage of bases (or amino acids) are the same in comparing the two sequences. It is noted that, for any sequence (“reference sequence”) disclosed in this application, sequences having at least about 60%, or at least about 65%, or at least about 70%, or at least about 75%, or at least about 80%, or at least about 83%, or at least about 85%, or at least about 90%, or at least about 95%, or at least about 98% or at least about 99% sequence identity to the reference sequence are also within the disclosure.

Likewise, the present disclosure also includes sequences that have one, two, three, four, or five substitution, deletion or addition of amino acid residues or nucleotides as compared to the reference sequences.

In any of the embodiments described herein, analogs of a peptide comprising any amino acid sequence described herein are also provided, which have at least about 80%, or at least about 83%, or at least about 85%, or at least about 90%, or at least about 95%, or at least about 98%, or at least about 99% sequence identity to any of reference amino acid sequences. In some embodiments, the analogs include one, two, three, four, or five substitution, deletion or addition of amino acid residues as compared to the reference sequences. In some embodiments, the substitution is a conservative substitution.

As used herein the term “amino acid” refers to either natural and/or unnatural or synthetic amino acids, including glycine and both the D and L optical isomers, amino acid analogs and peptidomimetics. In some embodiments, non-natural amino acids are useful for tuning or engineering the helix or other secondary or tertiary structures of a peptide or protein for desired antimicrobial properties.

As is well-known in the art, a “conservative substitution” of an amino acid or a “conservative substitution variant” of a peptide refers to an amino acid substitution which maintains: 1) the secondary structure of the peptide; 2) the charge or hydrophobicity of the amino acid; and 3) the bulkiness of the side chain or any one or more of these characteristics. Illustratively, the well-known terminologies “hydrophilic residues” relate to serine or threonine. “Hydrophobic residues” refer to leucine, isoleucine, phenylalanine, valine or alanine, or the like. “Positively charged residues” relate to lysine, arginine, ornithine, or histidine. “Negatively charged residues” refer to aspartic acid or glutamic acid. Residues having “bulky side chains” refer to phenylalanine, tryptophan or tyrosine, or the like. A list of illustrative conservative amino acid substitutions is given in Table A.

TABLE A

| For | |
|---|---|
| Amino | |
| Acid | Replace With |
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu |
| | Gln, D-Gln |
| Aspartic | D-Asp, D-Asn, Asn, Glu, D-Glu, |
| Acid | Gln, D-Gln |
| Cysteine | D-Cys, S-Me-Cys, Met, D-Met, Thr, |
| | D-Thr, L-Ser, D-Ser |
| Glutamine | D-Gln, Asn, D-Asn, Glu, D-Glu, |
| | Asp, D-Asp |
| Glutamic | D-Glu, D-Asp, Asp, Asn, D-Asn, |
| Acid | Gln, D-Gln |
| Glycine | Ala, D-Ala, Pro, D-Pro, Aib, β-Ala |
| Isoleucine | D-Ile, Val, D-Val, Leu, |
| | D-Leu, Met, D-Met |
| Leucine | Val, D-Val, Met, D-Met, D-Ile, |
| | D-Leu, Ile |
| Lysine | D-Lys, Arg, D-Arg, Orn, D-Orn |
| Methionine | D-Met, S-Me-Cys, Ile, D-Ile, Leu, |
| | D-Leu, Val, D-Val |
| Phenylalanine | D-Phe, Tyr, D-Tyr, His, |
| | D-His, Trp, D-Trp |
| Proline | D-Pro |
| Serine | D-Ser, Thr, D-Thr, allo-Thr, L-Cys, |
| | D-Cys |
| Threonine | D-Thr, Ser, D-Ser, allo-Thr, Met, |
| | D-Met, Val, D-Val |
| Tyrosine | D-Tyr, Phe, D-Phe, His, |
| | D-His, Trp, D-Trp |
| Valine | D-Val, Leu, D-Leu, Ile, |
| | D-Ile, Met, D-Met |

Alternatively, non-limiting examples of conservative amino acid substitutions are provided in Table B below, where a similarity score of 0 or higher indicates conservative substitution between the two amino acids.

TABLE B

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

Alternatively, non-limiting examples of conservative amino acid substitutions include substitutions of a polar amino acid with a different polar amino acid, or substitutions of a hydrophobic amino acid with a different hydrophobic amino acid, as illustrated in Table C below. Each of the polar amino acids or hydrophobic amino acids, in some embodiments, can be substituted with Ala or Gly.

TABLE C

| | |
|---|---|
| Polar amino acids | K, R, H, E, D, N, Q, S, T |
| | (or substituted with A or G) |
| Hydrophobic amino acids | V, I, L, M, C, F, W, Y |
| | (or substituted with A or G) |

As used herein, the term “composition” refers to a preparation suitable for administration to an intended patient for therapeutic purposes that contains at least one pharmaceutically active ingredient, including any solid form thereof. The composition may include at least one pharmaceutically acceptable component to provide an improved formulation of the compound, such as a suitable carrier. In certain embodiments, the composition is formulated as a film, gel, patch, or liquid solution.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

### 2. Antimicrobial Type II Bacteriocins

The present disclosure, in some embodiments, describes a computational approach for generating a systemic formula and related procedure to screen for new antimicrobial type-II bacteriocins. The identified protein and peptide families and their specific sequences are provided in Tables 2 and 3 and the appended Sequence Listing (SEQ ID NO:1-8, 9-327 and 328-1884). In some embodiments, provided is an isolated peptide comprising an amino acid sequence of Table 2 or 3 or the appended Sequence Listing (SEQ ID NO:1-1884) or an amino acid derived therefrom with one, two or three amino acid substitution. In some embodiments, the substitution is a conservative substitution. In some embodiments, the substitution is the replacement of a polar amino acid with a different polar amino acid, or the replacement of a hydrophobic amino acid with a different hydrophobic amino acid.

In some embodiments, provided is an isolated peptide comprising an amino acid sequence selected from the group consisting of EWGKQLGKWIGSKIK (SEQ ID NO: 1), KYVKNYLDFIKKAIDIFRPMPI (SEQ ID NO: 2), KWAKSAGKWIASKIK (SEQ ID NO: 3), KIGKTVGTIVRKGFEIWSIFK (SEQ ID NO: 4), and an amino acid derived from SEQ ID NO: 1, 2, 3 or 4 with one, two or three amino acid substitution. In some embodiments, the peptide comprises an amino acid sequence selected from the group consisting of FKVIVTDAGHYPREWGKQLGKWIGSKIK (SEQ ID NO: 5), KRNYSIEKYVKNYIDFIKKAIDIFRPMPI (SEQ ID NO: 6), KTIATNATYYPNKWAKSAGKWIASKIK (SEQ ID NO: 7), QYDKTGYKIGKTVGTIVRKGFEIWSIFK (SEQ ID NO: 8), and an amino acid derived from SEQ ID NO: 5, 6, 7, or 8 with one amino acid substitution. In some embodiments, the substitution is a conservative substitution. In some embodiments, the substitution is the replacement of a polar amino acid with a different polar amino acid, or the replacement of a hydrophobic amino acid with a different hydrophobic amino acid.

In some embodiments, the peptide is a fragment or fusion peptide described from natural proteins. In some embodiments, the peptide differs from natural proteins by at least an amino acid substation, addition or deletion.

In some embodiments, the peptide is not longer than 100 amino acid residues in length. In some embodiments, the peptide is not longer than 95, 90, 85, 80, 75, 70, 65, 60, 55, 50, 45, 40, 35, 30 or 25 amino acid resides in length. In some embodiments, the peptide has antimicrobial activity.

In some embodiments, the peptide has antimicrobial activity. In some embodiments, the peptides may be conjugated to therapeutic agents, prodrugs, peptides, proteins, enzymes, viruses, lipids, biological response modifiers, pharmaceutical agents, or PEG.

The peptides may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art. The peptides can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

### 3. Synthesis of Antimicrobial Peptides

The peptides described herein can be ordered from a commercial source or partially or fully synthesized using methods well known in the art (e.g., chemical and/or biotechnological methods). In certain embodiments, the peptides are synthesized according to solid phase peptide synthesis protocols that are well known in the art. In another embodiment, the peptide is synthesized on a solid support according to the well-known Fmoc protocol, cleaved from the support with trifluoroacetic acid and purified by chromatography according to methods known to persons skilled in the art. In other embodiments, the peptide is synthesized utilizing the methods of biotechnology that are well known to persons skilled in the art. In one embodiment, a DNA sequence that encodes the amino acid sequence information for the desired peptide is ligated by recombinant DNA techniques known to persons skilled in the art into an expression plasmid (for example, a plasmid that incorporates an affinity tag for affinity purification of the peptide), the plasmid is transfected into a host organism for expression, and the peptide is then isolated from the host organism or the growth medium, e.g., by affinity purification.

The peptides can be also prepared by using recombinant expression systems. Generally, this involves inserting the nucleic acid molecule into an expression system to which the molecule is heterologous (i.e., not normally present). One or more desired nucleic acid molecules encoding a peptide of the disclosure may be inserted into the vector. When multiple nucleic acid molecules are inserted, the multiple nucleic acid molecules may encode the same or different peptides. The heterologous nucleic acid molecule is inserted into the expression system or vector in proper sense (5′→3′) orientation relative to the promoter and any other 5′ regulatory molecules, and correct reading frame.

Purified peptides may be obtained by several methods. The peptide is preferably produced in purified form (preferably at least about 80% or 85% pure, more preferably at least about 90% or 95% pure) by conventional techniques. Depending on whether the recombinant host cell is made to secrete the peptide into growth medium (see U.S. Pat. No. 6,596,509 to Bauer et al., which is hereby incorporated by reference in its entirety), the peptide can be isolated and purified by centrifugation (to separate cellular components from supernatant containing the secreted peptide) followed by sequential ammonium sulfate precipitation of the supernatant. The fraction containing the peptide is subjected to gel filtration in an appropriately sized dextran or polyacrylamide column to separate the peptides from other proteins. If necessary, the peptide fraction may be further purified by HPLC.

### 4. Antimicrobial Compositions and Formulations

Compositions and formulations that include any one or more of the peptides as disclosed herein are also provided. In one embodiment, the composition includes any one or more of the peptides and a pharmaceutically acceptable carrier.

“Pharmaceutically acceptable carriers” refers to any diluents, excipients, or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The pharmaceutical compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly (ethyleneglycol), petroleum hydrocarbons, such as mineral oil and petrolatum, and water may also be used in suspension formulations.

The compositions of this disclosure are formulated for pharmaceutical administration to a mammal, preferably a human being. Such pharmaceutical compositions of the disclosure may be administered in a variety of ways, preferably parenterally.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

In addition to dosage forms described above, pharmaceutically acceptable excipients and carriers and dosage forms are generally known to those skilled in the art and are included in the disclosure. It should be understood that a specific dosage and treatment regimen for any particular patient will depend upon a variety of factors, including the activity of the specific peptide employed, the age, body weight, general health, sex and diet, renal and hepatic function of the patient, and the time of administration, rate of excretion, drug combination, judgment of the treating physician or veterinarian and severity of the particular disease being treated.

In some embodiments, the composition can further include a secondary antimicrobial agent. Non-limiting examples of such agents include imipenem, ceftazidime, colistin, chloroquine, artemisinin, vancomycin and daptomycin.

### 5. Therapeutic Methods

Methods of using the peptides, compositions and formulations of the present disclosure are also described. In one embodiment, the methods are for preventing or treating an infection of a microorganism. The microorganism can be a bacterium, such as a Gram-negative bacterium or a Gram-positive bacterium, a fungus, or a parasite.

The peptides, compositions and formulations are also useful for treating a disease or condition associated with an infection, such as wound abscess, catheter biofilm, pneumonia, and bacteremia.

In some embodiments, the treatment methods further include administration, concurrently or sequentially, of a second secondary antimicrobial agent. Non-limiting examples of such agents include imipenem, ceftazidime, colistin, chloroquine, artemisinin, vancomycin and daptomycin.

The peptides, compositions and formulations of the disclosure may be administered to the systemic circulation via parental administration. The term “parenteral” as used herein includes subcutaneous, intravenous, intramuscular, intra-articular, intra-synovial, intrasternal, intrathecal, intrahepatic, intralesional and intracranial injection or infusion techniques. However, in cases where the infection is local (e.g., on the skin), the composition may be administered locally, such as topically.

### 6. Computational System and Methods

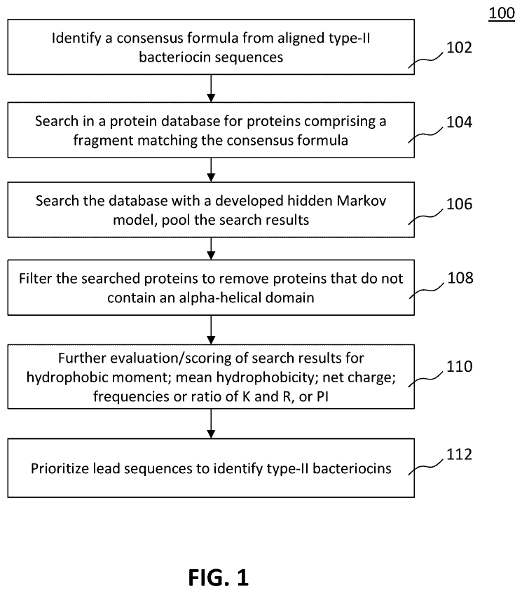

The present disclosure, in some embodiments, provides computer-implemented methods for identifying antimicrobial sequences and related systems and non-transitory computer-readable media. In one embodiment, a computer-implemented method of identifying a type-II bacteriocin is provided, as illustrated in FIG. 1 which is a process flow chart of a method 100. The various processing operations and/or data flows depicted in FIG. 1 (and in the other drawing figures) are described in greater detail herein. The described operations may be accomplished using some or all of the system components described in detail above and, in some implementations, various operations may be performed in different sequences and various operations may be omitted. Additional operations may be performed along with some or all of the operations shown in the depicted flow diagrams. One or more operations may be performed simultaneously. Accordingly, the operations as illustrated (and described in greater detail below) are exemplary by nature and, as such, should not be viewed as limiting.

In one embodiment, provided is a computer-implemented method of identifying a type-II bacteriocin. Optionally, at the first step, the method entails identifying a consensus formula from aligned type-II bacteriocin sequences (step 102). In a preferred embodiment, the consensus formula is not stringent so that more search results will be returned, which can be further filtered or screened. In some embodiment, the consensus formula comprises [LI]-[KREDNQSTYH]-X-[KREDNQSTYH]-X-[MLV]-X-X-[IVLT]-X-G-G, wherein X denotes any amino acid.

At step 104, the system or method searches in a protein database for proteins comprising a fragment matching the consensus formula for type-II bacteriocin (step 104). The protein database can be any database, data file, or data source that includes protein sequences. There are a number of publicly accessible protein databases available online. Matching a sequence to a formula can be done with methods known in the art.

At step 106, optionally, a hidden Markov model which can be developed based on known type-II bacteriocin sequences, can be used to search the database (step 106), in particular for unknown sequences. Methods of developing hidden Markov models and using them for sequence searches are known in the art. The search results from the hidden Markov model-based search can be pooled to the first search result.

It is the discovered herein that alpha-helical domain frequently co-exists with type-II bacteriocin domains in proteins. In some embodiments, accordingly, the search results are filtered to remove proteins that do not contain an alpha-helical domain (step 108). For instance, the alpha-helical domains can be identified with a consensus formula comprising X-[VILMCFWYAG]-[KRHEDNQSTAG]-[KRHEDNQSTAG]-[VILMCFWYAG]-[VILMCFWYAG]-[KRHEDNQSTAG]-[KRHEDNQSTAG]-[VILMCFWYAG]-X-[KRHEDNQSTAG]-[VILMCFWYAG], wherein X denotes any amino acid residue.

At step 110, optimally, a score can be calculated for the search results based on one or more parameters selected from hydrophobic moment, mean hydrophobicity, net charge, frequencies or ratio of K and R, or isoelectric point (step 110). In some embodiments, at least the hydrophobic moment is calculated. In some embodiments, at least the isoelectric point is calculated. In some embodiments, at least hydrophobic moment and isoelectric point are calculated, which can be optionally used for prioritizing, ranking, or selecting suitable type-II bacteriocins from the search results (step 112). Although not shown in FIG. 1, the selected type-II bacteriocins can be synthesized and tested in the lab.

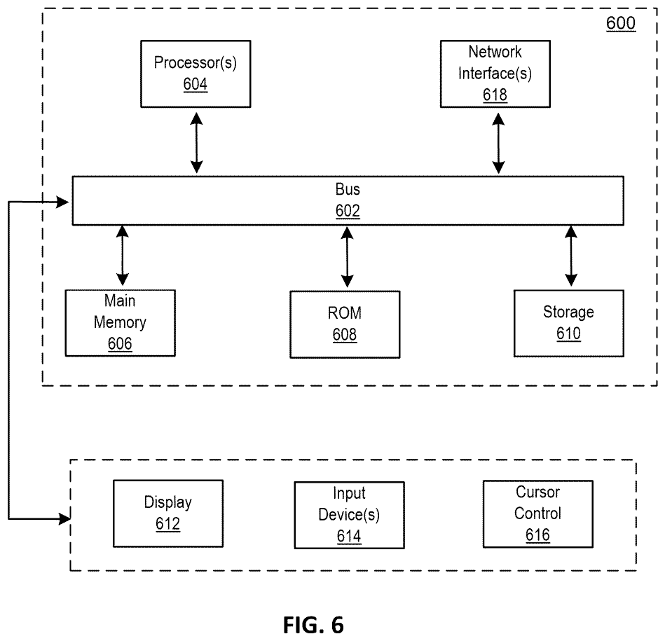

FIG. 6 depicts a block diagram of an example computer system 600 in which any of the embodiments described herein may be implemented. The computer system 600 includes a bus 602 or other communication mechanism for communicating information, one or more hardware processors 604 coupled with bus 602 for processing information. Hardware processor(s) 604 may be, for example, one or more general purpose microprocessors.

The computer system 600 also includes a main memory 606, such as a random access memory (RAM), cache and/or other dynamic storage devices, coupled to bus 602 for storing information and instructions to be executed by processor 604. Main memory 606 also may be used for storing temporary variables or other intermediate information during execution of instructions to be executed by processor 604. Such instructions, when stored in storage media accessible to processor 604, render computer system 600 into a special-purpose machine that is customized to perform the operations specified in the instructions.

The computer system 600 further includes a read only memory (ROM) 608 or other static storage device coupled to bus 602 for storing static information and instructions for processor 604. A storage device 610, such as a magnetic disk, optical disk, or USB thumb drive (Flash drive), etc., is provided and coupled to bus 602 for storing information and instructions.

The computer system 600 may be coupled via bus 602 to a display 612, such as a cathode ray tube (CRT) or LCD display (or touch screen), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

The computing system 600 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage.

The computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor(s) 604 executing one or more sequences of one or more instructions contained in main memory 606. Such instructions may be read into main memory 606 from another storage medium, such as storage device 610. Execution of the sequences of instructions contained in main memory 606 causes processor(s) 604 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 610. Volatile media includes dynamic memory, such as main memory 606. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may retrieves and executes the instructions. The instructions received by main memory 606 may optionally be stored on storage device 610 either before or after execution by processor 604.

The computer system 600 also includes a communication interface 618 coupled to bus 602. Communication interface 618 provides a two-way data communication coupling to one or more network links that are connected to one or more local networks. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN (or WAN component to communicated with a WAN). Wireless links may also be implemented. In any such implementation, communication interface 618 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet”. Local network and Internet both use electrical, electromagnetic or optical signals that carry digital data streams. The signals through the various networks and the signals on network link and through communication interface 618, which carry the digital data to and from computer system 600, are example forms of transmission media.

The computer system 600 can send messages and receive data, including program code, through the network(s), network link and communication interface 618. In the Internet example, a server might transmit a requested code for an application program through the Internet, the ISP, the local network and the communication interface 618.

The received code may be executed by processor 604 as it is received, and/or stored in storage device 610, or other non-volatile storage for later execution.

Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

### Engines, Components, and Logic

Certain embodiments are described herein as including logic or a number of components, engines, or mechanisms. Engines may constitute either software engines (e.g., code embodied on a machine-readable medium) or hardware engines. A “hardware engine” is a tangible unit capable of performing certain operations and may be configured or arranged in a certain physical manner. In various example embodiments, one or more computer systems (e.g., a standalone computer system, a client computer system, or a server computer system) or one or more hardware engines of a computer system (e.g., a processor or a group of processors) may be configured by software (e.g., an application or application portion) as a hardware engine that operates to perform certain operations as described herein.

In some embodiments, a hardware engine may be implemented mechanically, electronically, or any suitable combination thereof. For example, a hardware engine may include dedicated circuitry or logic that is permanently configured to perform certain operations. For example, a hardware engine may be a special-purpose processor, such as a Field-Programmable Gate Array (FPGA) or an Application Specific Integrated Circuit (ASIC). A hardware engine may also include programmable logic or circuitry that is temporarily configured by software to perform certain operations. For example, a hardware engine may include software executed by a general-purpose processor or other programmable processor. Once configured by such software, hardware engines become specific machines (or specific components of a machine) uniquely tailored to perform the configured functions and are no longer general-purpose processors. It will be appreciated that the decision to implement a hardware engine mechanically, in dedicated and permanently configured circuitry, or in temporarily configured circuitry (e.g., configured by software) may be driven by cost and time considerations.

Accordingly, the phrase “hardware engine” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented engine” refers to a hardware engine. Considering embodiments in which hardware engines are temporarily configured (e.g., programmed), each of the hardware engines need not be configured or instantiated at any one instance in time. For example, where a hardware engine comprises a general-purpose processor configured by software to become a special-purpose processor, the general-purpose processor may be configured as respectively different special-purpose processors (e.g., comprising different hardware engines) at different times. Software accordingly configures a particular processor or processors, for example, to constitute a particular hardware engine at one instance of time and to constitute a different hardware engine at a different instance of time.

Hardware engines can provide information to, and receive information from, other hardware engines. Accordingly, the described hardware engines may be regarded as being communicatively coupled. Where multiple hardware engines exist contemporaneously, communications may be achieved through signal transmission (e.g., over appropriate circuits and buses) between or among two or more of the hardware engines. In embodiments in which multiple hardware engines are configured or instantiated at different times, communications between such hardware engines may be achieved, for example, through the storage and retrieval of information in memory structures to which the multiple hardware engines have access. For example, one hardware engine may perform an operation and store the output of that operation in a memory device to which it is communicatively coupled. A further hardware engine may then, at a later time, access the memory device to retrieve and process the stored output. Hardware engines may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented engines that operate to perform one or more operations or functions described herein. As used herein, “processor-implemented engine” refers to a hardware engine implemented using one or more processors.

Similarly, the methods described herein may be at least partially processor-implemented, with a particular processor or processors being an example of hardware. For example, at least some of the operations of a method may be performed by one or more processors or processor-implemented engines. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across a number of machines. In some example embodiments, the processors or processor-implemented engines may be located in a single geographic location (e.g., within a home environment, an office environment, or a server farm). In other example embodiments, the processors or processor-implemented engines may be distributed across a number of geographic locations.

### Language

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the subject matter has been described with reference to specific example embodiments, various modifications and changes may be made to these embodiments without departing from the broader scope of embodiments of the present disclosure. Such embodiments of the subject matter may be referred to herein, individually or collectively, by the term “invention” merely for convenience and without intending to voluntarily limit the scope of this application to any single disclosure or concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will be appreciated that an “engine,” “system,” “data store,” and/or “database” may comprise software, hardware, firmware, and/or circuitry. In one example, one or more software programs comprising instructions capable of being executable by a processor may perform one or more of the functions of the engines, data stores, databases, or systems described herein. In another example, circuitry may perform the same or similar functions. Alternative embodiments may comprise more, less, or functionally equivalent engines, systems, data stores, or databases, and still be within the scope of present embodiments. For example, the functionality of the various systems, engines, data stores, and/or databases may be combined or divided differently.

“Open source” software is defined herein to be source code that allows distribution as source code as well as compiled form, with a well-publicized and indexed means of obtaining the source, optionally with a license that allows modifications and derived works.

The data stores described herein may be any suitable structure (e.g., an active database, a relational database, a self-referential database, a table, a matrix, an array, a flat file, a documented-oriented storage system, a non-relational No-SQL system, and the like), and may be cloud-based or otherwise.

### EXAMPLES

### Example 1. Search and Testing of Type-II Bacteriocins

This example describes a method of searching for type-II bacteriocins and confirmation of the identified type-II bacteriocins having microbiocidal activities.

Among the earliest antimicrobial compounds first arose in prokaryotes themselves as means to defend against microbial competitors. Of these, well characterized members include class II bacteriocins, ribosomally-synthesized polypeptides produced primarily by Gram-positive bacteria. While a number of class II bacteriocins have been described, an expanding universe of bioinformatic datasets affords means by which to identify novel bacteriocin sequences from newly sequenced genomes. In the current investigation, a new search method was created to rapidly identify bacteriocin sequences with a high degree of fidelity. This multi-component method integrates the application of a relaxed bacteriocin signal peptide search term along with an amphipathic pattern search tool as a means to select for bacteriocin-like sequences. When applied, this method accurately identifies virtually all families of known class II bacteriocins. Moreover, the method retrieved a large number of sequences having structural features characteristic of ancient antimicrobial bacteriocins. Selected sequences identified by this search were synthesized and demonstrated to exert potent antimicrobial efficacy in vitro against a broad spectrum of human pathogens. These results represent discovery of new sequences and putative subclasses of type II bacteriocin may serve as physicochemical templates for innovative anti-infective therapeutic agents to meet the challenge of antibiotic resistance.

### Methods and Materials

### Identification of the Type IIa Bacteriocin Consensus Formula

To identify a consensus formula that was consistent with a majority of known class IIa bacteriocins, multiple sequence alignments with prototypic representatives of this family were carried out using CLUSTAL W, and further analyzed using MEGA 6. Sites of potential conservation were scored for specific residue or chemical identity to generate a 12 residue consensus formula. Several positions within the formula were degenerate depending on sequence or chemical (polar residues) conservation at individual sites.

### Apply Method to Identify Putative Bacteriocins

Once this consensus formula was created, it was used with ScanProsite (**) to carry out a pattern search of the UniProtKB/Swiss-Prot and UniProtKB/TrEMBL databases. Search results were further limited as follows: 1) protein size (<80 residues); 2) bacterial organisms; and 3) localization of the pattern to the first 25 residues of the protein with a “<X(0,25)” logical operator.

### Search for Amphipathic α-Helices Within Retrieved Dataset

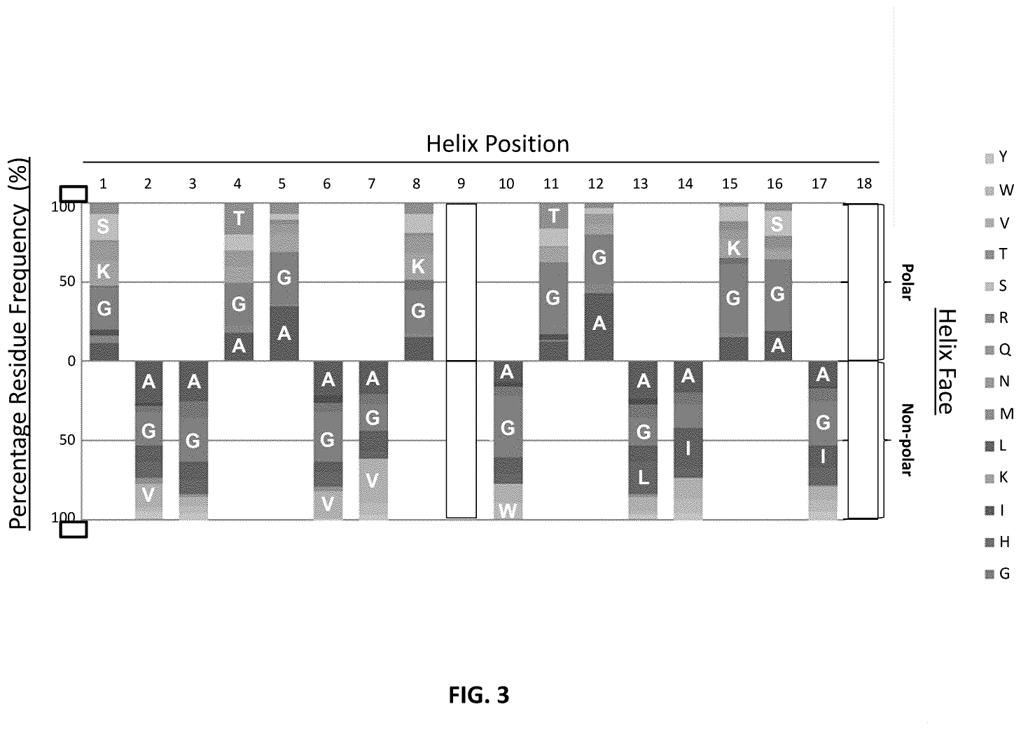

Results from the above database search were then formatted as per UniProtKB Swiss-Prot requirements and submitted as a sequence database against which additional pattern searches could be carried out. This database was then queried with a degenerate amphipathic sequence formula to scan for α-helical domains within retrieved protein dataset. The formula was advanced one position at a time through 18 iterations to represent an entire 18-residue helical wheel span (FIG. 3). Iteration 1 of this query sequence is listed below:

X-[VILMCFWYAG]-[KRHEDNQSTAG]-[KRHEDNQSTAG]-
[VILMCFWYAG]-[VILMCFWYAG]-[KRHEDNQSTAG]-
[KRHEDNQSTAG]-[VILMCFWYAG]-X[KRHEDNQSTAG]-
[VILMCFWYAG]

As the mature bacteriocin peptide is usually located near the C-terminus of the protein, search parameters included a “X(0,30)>” logical operator to restrict results to the final 30 residues of the protein.

### Biophysical Parameter Determination

Retrieved datasets were subjected to batch analysis to compute the PI of individual sequences using ExPasy Compute PI/MW tool. Hydrophobic moment, mean hydrophobicity, net charge [K and R (+1), H (+0.5), D and E (−1)] and K and R residue frequency were determined using Python programs created for this purpose.

### Operon Characterization

To probe for new, as yet unidentified, bacteriocins, the genomic regions surrounding uncharacterized hits were analyzed. A total of 20,000 base pairs (10,000 upstream and 10,000 downstream) from the identified sequence were scored for the presence of typical bacteriocin operon genes (ABC transporters, immunity proteins, pheromones) using the Ensembl Genomes REST API. Sequences that were found to be closely associated with bacteriocin-operon genes were prioritized for further study.

### Peptide Synthesis

Select candidate microbicidal peptides were commercially synthesized by BioMatik at a level of >98% purity. Lyophilized peptides were reconstituted with ddIH20 and stored in aliquots at −20° C.

### Assay for Antimicrobial Activity

Antimicrobial assays were performed using a well-established radial diffusion method modified to pH 5.5 or 7.5. A panel of microorganisms was tested: Gram-positive Staphylococcus aureus (ISP 479C, ISP 479R); Gram-negative Salmonella typhimurium (MS 5996s, MS 14028), Pseudomonas aeruginosa (PA01), Acinetobacter baumanni (17928) and the fungus Candida albicans (36082S, 36082R). Logarithmic phase organisms were inoculated (10⁶ CFU/ml) into buffered agarose, and poured into plates. Peptides (10 μg) were introduced into wells in the seeded matrix, and incubated for 3 h at 37° C. Nutrient overlay medium was applied, and assays incubated at 37° C. or 30° C. for bacteria or fungi, respectively. After 24 h, zones of inhibition were measured. Independent experiments were repeated a minimum of two times.

### Results

### Identification of a Class II Bacteriocin Consensus Sequence Formula

To identify a signal peptide consensus formula that was consistent with a majority of known Class II (a-d) bacteriocins, multiple sequence alignments with prototypic representatives from these families were carried out. Initial alignments were generated using CLUSTAL W, followed by manual adjustment to align the double glycine motif using MEGA 6.

Analysis of these alignments revealed a number of conserved residues within the signal peptide domain that were used to generate a 12 residue consensus formula as follows:

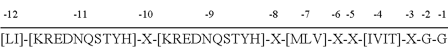

As several positions within these alignments were conserved only at the physicochemical level (positions-9 and -11), they are represented by degenerate search terms reflecting the likelihood of a polar residue at these positions.

### Primary Screen: Signal Peptide Method

The above formula was used as a ProSite query against the UniProtKB/Swiss-Prot and/TrEMBL databases and retrieved a total of 3050 sequences. Within this dataset the following classes of bacterial proteins were represented: 376 bacteriocins; 45 putative bacteriocins; 129 competence enhancing peptides; 7 pheromones; 12 autoinducing peptides; 170 putative bacteriocin related proteins; 182 other proteins; and 2129 uncharacterized sequences. All of the sequences are provided in the appended Sequence Listing, while a number of representative sequences are shown in Table 3 below.

In general, the majority of all sequences (bacteriocins and non-bacteriocins) retrieved with the signal peptide consensus were from Gram-positive Firmicutes (64%) and other Gram-positive organisms (Actinobacteria [2%], Deinococci [>1%]). Sequences were also retrieved from a number of Gram-negative organisms (Bacteroidetes [Chlorobi group, 13%], Chlamydia [>1%], Chloracidobacterium [>1%], Cyanobacteria [3%], Dehalococcoides [>1%], Fusobacteriales [>1%], Planctomycetia [>1%], Proteobacteria [17%]).

With respect to specificity, the formula retrieved a high percentage of the known Class II bacteriocin sequences. Notably, members from nearly all of the of Class IIa and IIb families were retrieved by the search. In particular, the formula identified representatives from approximately 90% of Class IIa families and 88% of Class IIb families. By comparison, many fewer of the cyclic, (13%) Class IIc, and other, (13%) Class IId, peptide groups were retrieved (Table 1). For many of the listed bacteriocins more than one representative of each family was retrieved. In some cases a large number of family members were retrieved such as for the class IIb Lactobin family where more than 90 members were identified.

TABLE 1
Bacteriocin Peptides Retrieved by Signal Consensus Formula Search

| Class | Peptide | | Organism |
|---|---|---|---|
| IIa | Acidocin | 8912, LF221B, M | Lactobacillus acidophilus |
| | Avicin | A | Enterococcus avium (Streptococcus avium) |
| | Carnobacteriocin | A, B2, BM1 | Carnobacterium maltaromaticum |
| | Curvacin | A | Lactobacillus curvatus |
| | Divergicin | 750 | Carnobacterium divergens (Lactobacillus divergens) |
| | Enterocin | B, 1071A/1B, CRL35, | Enterococcus faecium |
| | | C2, NKR-5-3A/3Z, HF, | |
| | | Xbeta | |
| | Leucocin | A, B, K, N, Q | Leuconostoc gelidum, Leconostoc carnosum, |
| | Mundticin | KS, L | Enterococcus pallens ATCC BAA-351 |
| | PapA | | Listeria aquatica FSL S10-1188 |
| | Piscicolin | 126 | Carnobacterium maltaromaticum |
| | Plantaricin | A, F, J, 1.25 beta, c81F | Lactobacillus plantarum |
| | Sakacin | A, D98c, P, X | Lactobacillus sakei |
| IIb | Amylovorin | L alpha, L beta | Lactobacillus amylovorus |
| | Bacteriocin | GatX, BacSJ2-8 | Streptococcus pneumoniae |
| | Brevicin | 925A | Lactobacillus brevis |
| | Gassericin | T | Lactobacillus gasseri |
| | Lactobin/Cerein | A/7B | Streptococcus australis ATCC 700641 |
| | Lactocin | 705 alpha, 705 beta | Lactobacillus curvatus |
| | Lactacin | F | Lactobacillus johnsonii |
| IId | Lactococcin | G | Clostridium perfringens (strain SM101/Type A) |
| | Mesentericin | B105, Y105 | Leuconostoc mesenteroides |
| | Weissellicin | L | Weissella hellenica |

### Secondary Screen: Amphipathic Helix Method

As a refinement of this search, the retrieved sequences from the primary scan were formatted as a database, and a secondary screen with the amphipathic helix formula was carried out. In this case, the search was targeted to the final 30 residues of the peptide to probe for the presence of this motif within the mature portion of the peptide. When compared with the primary screen, this secondary screen provided information regarding the likelihood that retrieved peptides were α-helical in nature.

With this refinement, the total number of retrieved sequences was 1557 representing: 302 bacteriocins; 27 putative bacteriocins; 31 competence enhancing peptides; 1 pheromone; 11 autoinducing peptides; 78 putative bacteriocin related proteins; 65 other proteins; and 1042 uncharacterized sequences. Notably, while fewer sequences were retrieved with this secondary screen, the relative percentage of known bacteriocins within the characterized protein sub-dataset was increased from 41% to 53% of the returned sequences.

When the secondary alpha helical screen was applied to the dataset, nearly all of the Class IIa and IIb bacteriocin families were retrieved, suggesting that most of these peptides may be capable of adopting an α-helical conformation in membrane mimetic environments. One notable exception to this observation was for the pediocin sequences, which were not retrieved by the amphipathic sequence formula. However, this result may be expected, as many members of the pediocin-like bacteriocin group have been shown to form a hairpin-like structure at the C-terminus.

Residue Frequency within Bacteriocins

Given that the amphipathic sequence formula retrieves aligned datasets, peptides can be scored for the abundance of individual residues along an amphipathic helical span. When these alignments are carried out, the abundance of individual residues along both the predicted polar and non-polar faces of the retrieved helices can be calculated. In these studies, such alignments indicated that glycine was the most abundant residue at nearly all positions of these putative helical spans, whether present on the hydrophobic or hydrophilic face of the helix. In the few positions where glycine was not the most abundant residue, often the next most tiny and conformationally flexible residue, alanine, was present.

On the polar face of the peptide, after glycine and alanine the next most abundant residues were the cationic residue lysine and neutral hydrophilic residues threonine and serine. Notably, the cationic residue arginine was rarely found on the polar face of these bacteriocin amphipathic spans.

On the non-polar face the most abundant residues after glycine and alanine were valine, leucine and isoleucine. Tryptophan was also found with some abundance on this face at some positions, although tyrosine and phenylalanine were infrequently found in this analysis.

### Analysis of Uncharacterized Sequences

Besides retrieving a significant number of known bacteriocin sequences, the amphipathic sequence method also identified a large number (˜1042) of as yet uncharacterized sequences. If bacteriocins are represented to an equivalent degree between the characterized and uncharacterized datasets, it would be predicted that more than 550 of these unknown sequences may be novel bacteriocins (53% percent unknown=552).

As a means to characterize these unknown sequences, they were initially subjected to computational (Clustal W) and manual (MEGA 6) alignment (Supplemental Figure X). Unknown sequences were also scored for the following biophysical parameters: PI, net charge (Q), hydrophobic moment (μH), hydrophobicity (H) and arginine to lysine ratio (N_{K}/N_{K}+N_{R}).

Results from the above analyses were integrated, and four sequences (Table 2) from phylogenetically distinct organisms were chosen for further characterization (peptide name; SwissProt accession; species): peptide 1—A0RKV8 (Bacillus thuringiensis); peptide 2—D6E338 (Eubacterium rectale); peptide 3—B3ZXE9 (Bacillus cereus); peptide 4—R2S6C2 (Enterococcus pallens).

TABLE 2
Four identified peptide sequences, each along with a longer version,
which was synthesized and tested

| | | | | SEQ ID |
|---|---|---|---|---|
| No. | Name | Organism | Sequence | NO: |
| A0RKV8 | Uncharacterized | Bacillus | EWGKQLGKWI GSKIK | 1 |
| (Peptide 1) | protein | thuringiensis | | |
| | | (strain Al Hakam) | | |
| Peptide 1 - | | | FKVIVTDAGH YPREWGKQLG | 5 |
| longer version | | | KWIGSKIK | |
| D6E338 | Uncharacterized | [Eubacterium] | KYVKNYLDFI KKAIDIFRPM | 2 |
| (Peptide 2) | protein | rectale DSM 17629 | PI | |
| Peptide 2 - | | | KRNYSIEKYV KNYlDFIKKA | 6 |
| longer version | | | IDIFRPMPI | |
| B3ZXE9 | Uncharacterized | Bacillus cereus | KWAKSAGKWI ASKIK | 3 |
| (Peptide 3) | protein | 03BB108 | | |
| Peptide 3 - | | | KTIATNATYY PNKWAKSAGK | 7 |
| longer version | | | WIASKIK | |
| R2S6C2 | Bacteriocin-type | Enterococcus | KIGKTVGTIV RKGFEIWSIF | 4 |
| (Peptide 4) | signal sequence | pallens | K | |
| | | ATCC BAA-351 | | |
| Peptide 4 - | | | QYDKTGYKIG KTVGTIVRKG | 8 |
| longer version | | | FEIWSIFK | |

| | Count | Count | | Q | | HM18* | | | | | |
|---|---|---|---|---|---|---|---|---|---|---|---|
| No. | K | R | Q18 | Full | HM18 | Qfull | PI | HM*PI | H18 | Charge | μH |
| A0RKV8 | 4 | 0 | 3 | 3 | 0.58 | 1.7 | 10 | 5.81 | 0.33 | +3.5 | 0.48 |
| D6E338 | 4 | 1 | 3 | 3 | 0.67 | 2.03 | 5.18 | 3.52 | 0.39 | +4 | 0.41 |
| B3ZXE9 | 5 | 0 | 5 | 5 | 0.50 | 2.51 | 9.94 | 5.00 | 0.26 | +6 | 0.35 |
| R2S6C2 | 3 | 1 | 3 | 4 | 0.58 | 2.34 | 9.46 | 5.54 | 0.43 | +4 | 0.44 |

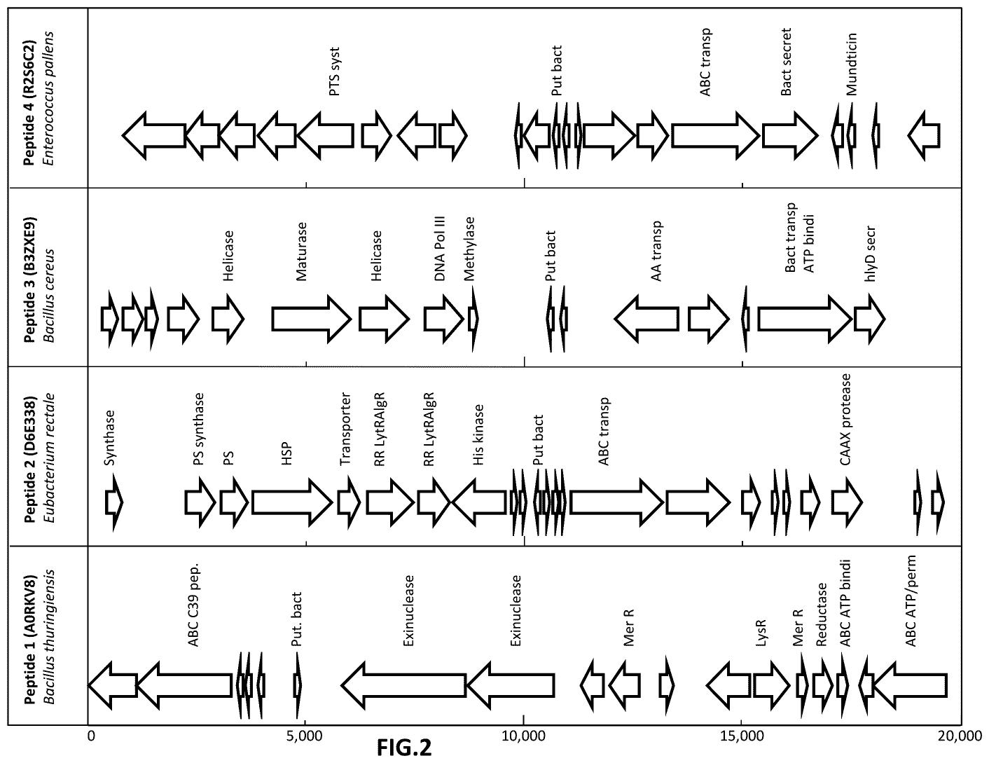

At the genetic level, peptides 1-4 were localized to bacteriocin-like operon regions containing ABC transporters and other bacteriocin-associated genes (FIG. 2). In particular, all were localized within 20 kb of an ABC transporter protein, as well as ABC transporter accessory genes such as C39 peptidases and ATP binding proteins. Many of the putative bacteriocins were also localized within clusters of small genes that resembled other bacteriocin sequences and/or pheromones. In some cases, prototypic bacteriocin immunity peptides were also localized within the putative bacteriocin operons.

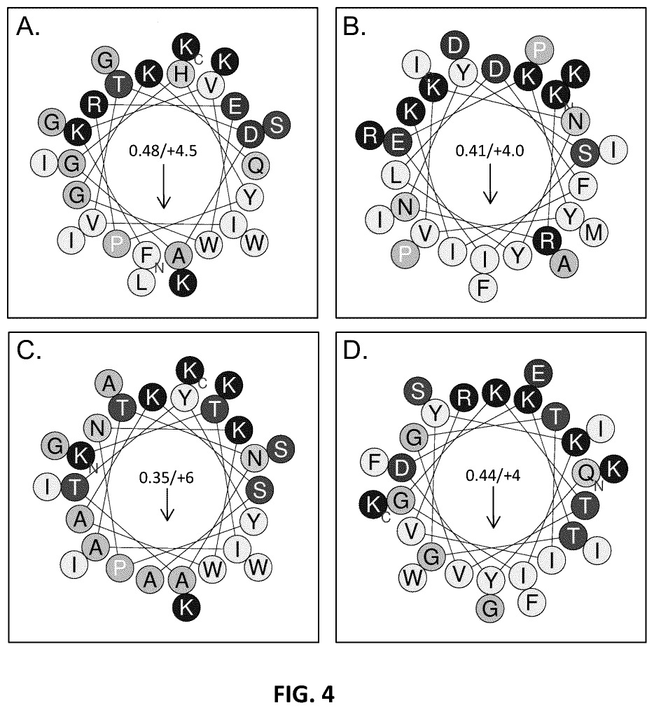

As a further means of characterization, peptides 1-4 were synthesized so that their microbicidal properties could be determined. This assessment revealed that all four putative bacteriocins possessed microbicidal activity against Gram positive (S. aureus), Gram negative (S. typhimurium, P. aeruginosa, A. baumanni) and fungal (C. albicans) organisms (FIG. 4). While active against all classes of microbes, peptides 1-4 were generally more potent towards Gram negative microorganisms than the other organisms in this study (FIG. 4).

As a test of the relative activity of these putative bacteriocins in blood versus phagolysosomal/wound septic environments, microbicidal assays were carried out in representative physiologic buffers at pH 7.5 and 5.5 respectively. In general, peptides 1-4 were typically more active at pH 7.5 than at pH 5.5, and notably peptide 3 lost nearly all of its activity against Gram positive S. aureus at pH 5.5.

Taken together, given their localization to bacteriocin-like operons and potent microbicidal spectrum, it seems likely that peptides 1-4 represent novel bacteriocin sequences. However, studies to isolate these putative sequences will be necessary to confirm this observation.

TABLE 3
Representative peptides identifies from the primary screen

| | | | | | | | SEQ |
|---|---|---|---|---|---|---|---|
| Accession | | | | | | | ID |
| # | Name | Comp Form Match | HM | Q | HMQ | PI | NO: |
| A1ZN33 | Uncharacterized protein | RQMVRHLRRFMRRYGRC | 0.70 | 7.5 | 5.27 | 7.93 | 9 |
| A0A0A1GSN1 | Uncharacterized protein | KNGLKKFFKWVRKL | 0.75 | 6 | 4.49 | 10.59 | 10 |
| D9SR34 | Uncharacterized protein | SLKKALKKAVSGLGKIIK | 0.66 | 6 | 3.99 | 9.6 | 11 |
| L7ZAD1 | Putative peptide | KTLTKFFKSLIKR | 0.72 | 5 | 3.58 | 10.36 | 12 |
| | pheromone/induction | | | | | | |
| | peptide | | | | | | |
| K9E5B5 | Uncharacterized protein | RCRKVYHRLWGK | 0.61 | 5.5 | 3.33 | 9.3 | 13 |
| D9SV22 | Uncharacterized protein | SLRKALKKIIDAIVK | 0.72 | 4 | 2.88 | 9.82 | 14 |
| K0NDW4 | Uncharacterized protein | SQLYKNLFKVFRK | 0.69 | 4 | 2.75 | 10.41 | 15 |
| I6WB56 | Putative plantaricin A | LQMGATAIKQVKKLFKKWG | 0.55 | 5 | 2.74 | 10.48 | 16 |
| | peptide | | | | | | |
| D1P3A0 | Uncharacterized protein | YKNVKAFIEKCRKVY | 0.64 | 4 | 2.54 | 9.24 | 17 |
| Q38Y68 | Putative bacteriocin | REYLNKITKWIKH | 0.71 | 3.5 | 2.48 | 9.92 | 18 |
| | inducing peptide | | | | | | |
| C2SB64 | Uncharacterized protein | WIGKVGKQYKKAT | 0.59 | 4 | 2.34 | 9.33 | 19 |
| R5IRG3 | Uncharacterized protein | GSIISSIVRGGILILELGRSFGSALRRLLKK | 0.45 | 5 | 2.24 | 10.01 | 20 |
| B1SE87 | Bacteriocin-type signal | RQAAKAIGKAVGKLF | 0.55 | 4 | 2.22 | 9.78 | 21 |
| | sequence | | | | | | |
| U2JCR9 | Bacteriocin-type signal | MRNGFEIFKKIAKSIIDAFRHA | 0.62 | 3.5 | 2.16 | 8.09 | 22 |
| | sequence | | | | | | |
| R2SSG8 | Bacteriocin-type signal | AVGKNVWEFGKGFVKGWNKAK | 0.51 | 4 | 2.02 | 10.09 | 23 |
| | sequence | | | | | | |
| R2S6C2 | Bacteriocin-type signal | KTGYKIGKTVGTIVRKGFEIWS | 0.50 | 4 | 2.01 | 9.46 | 24 |
| | sequence | | | | | | |
| E3R2A6 | Uncharacterized protein | GGSKGWNKFWKS | 0.64 | 3 | 1.93 | 9.52 | 25 |
| K1N8K9 | Uncharacterized protein | GGSKGWNKFWKS | 0.64 | 3 | 1.93 | 9.52 | 26 |
| D7V8H5 | Uncharacterized protein | GGGAWKNFWSSLRKGF | 0.62 | 3 | 1.86 | 9.99 | 27 |
| R9X316 | Uncharacterized protein | KQWYRLGERVGRVA | 0.62 | 3 | 1.85 | 9.69 | 28 |
| E7S5Z7 | Uncharacterized protein | GSACAYMRRVCKK | 0.46 | 4 | 1.84 | 9.1 | 29 |
| E8KCS4 | Bacteriocin-type signal | RRITSFFANLFQK | 0.61 | 3 | 1.84 | 9.99 | 30 |
| | sequence | | | | | | |
| A4BNK7 | Uncharacterized protein | QKAMKNIRGGARG | 0.44 | 4 | 1.77 | 11.91 | 31 |
| D3H665 | Excreted peptide | VKWCNKLFGTGRHVA | 0.50 | 3.5 | 1.74 | 4.43 | 32 |
| C2CZ62 | Bacteriocin-type signal | LSHISGGVTRYRHHEKKSWIDDFMKGFKK | 0.31 | 5.5 | 1.73 | 6.82 | 33 |
| | sequence | | | | | | |
| I0SA65 | Uncharacterized protein | SITNFWKKYFNH | 0.69 | 2.5 | 1.72 | 5.45 | 34 |
| I0T6B7 | Uncharacterized protein | SITNFWKKYFNH | 0.69 | 2.5 | 1.72 | 5.45 | 35 |
| R6SQB3 | Uncharacterized protein | YNPFKNIFHRFSNGH | 0.57 | 3 | 1.71 | 10.82 | 36 |
| K8WE53 | Uncharacterized protein | YKNVKAFIEKCRS | 0.56 | 3 | 1.69 | 9.17 | 37 |
| G0IAI7 | Uncharacterized protein | GRFFGGIYNAGRSFGRNV | 0.56 | 3 | 1.68 | 9.22 | 38 |
| A0A0E2PPW4 | Bacteriocin-type signal | GRFFGGIYNAGRSFGRNV | 0.56 | 3 | 1.68 | 9.22 | 39 |
| | sequence | | | | | | |
| G6A7G8 | Uncharacterized protein | RGIIGIGKKLFG | 0.55 | 3 | 1.66 | 9.7 | 40 |
| R7KCN7 | Uncharacterized protein | GTILNQLNKLISILVDSGKSLGSSIRRIS | 0.55 | 3 | 1.65 | 8.98 | 41 |
| A0A0B5ZQ16 | Uncharacterized protein | INGGKTIATNATYYPNKWAKSVGKWIAS | 0.41 | 4 | 1.63 | 9.94 | 42 |
| G6CHH9 | Bacteriocin-type signal | SNFIHKIKQIFTH | 0.54 | 3 | 1.62 | 9.82 | 43 |
| | sequence domain protein | | | | | | |
| Q5FI67 | Uncharacterized protein | IGGSAKSYIRRLG | 0.54 | 3 | 1.61 | 9.69 | 44 |
| R6Y309 | Uncharacterized protein | GSLFSALAKGFNVFVDMGRALGSSIRRLVNN | 0.53 | 3 | 1.58 | 9.18 | 45 |
| C0WN46 | Bacteriocin-type signal | LSHISGGVTRYRYHEKKSWIDDFMKGFKK | 0.32 | 5 | 1.58 | 6.82 | 46 |
| | sequence | | | | | | |
| C0XJC5 | Bacteriocin-type signal | LSHISGGVTRYRYHEKKSWIDDFMKGFKK | 0.32 | 5 | 1.58 | 6.82 | 47 |
| | sequence | | | | | | |
| R5Y709 | Uncharacterized protein | AAFINGVKGIFSIFFDFGNSLGSSIRRIK | 0.52 | 3 | 1.57 | 9.9 | 48 |
| U2YG00 | Uncharacterized protein | RGMIGIGKKLFG | 0.51 | 3 | 1.52 | 9.63 | 49 |
| R5IWG8 | Uncharacterized protein | ATFINAIVKGLSLIIELGKSLGSSFRRIT | 0.50 | 3 | 1.51 | 9.51 | 50 |
| B3ZXE9 | Uncharacterized protein | INGGKTIATNATYYPNKWAKSAGKWIAS | 0.38 | 4 | 1.51 | 9.94 | 51 |
| B6W9P3 | Bacteriocin-type signal | QIYKEAKDFVNNRAGNLIRGFKDVWKN | 0.49 | 3 | 1.48 | 9.1 | 52 |
| | sequence | | | | | | |
| A0A075JT42 | Uncharacterized protein | LSRVKGGMSRIAG | 0.49 | 3 | 1.46 | 9.3 | 53 |
| C2MRT0 | Uncharacterized protein | KWGKELGRWIGS | 0.73 | 2 | 1.46 | 10.09 | 54 |
| A0RKV8 | Uncharacterized protein | REWGKQLGKWIGS | 0.71 | 2 | 1.41 | 10 | 55 |
| G2G092 | Bacteriocin-type signal | SGGGKFIGNLIKLARP | 0.44 | 3 | 1.33 | 10.49 | 56 |
| | sequence domain protein | | | | | | |
| F9D2A0 | Uncharacterized protein | LRFLDRIMGGFRQ | 0.66 | 2 | 1.32 | 11.55 | 57 |
| A0A059KSD2 | Uncharacterized protein | FGGLLRHLFGFK | 0.52 | 2.5 | 1.31 | 9.9 | 58 |
| S3ZDV9 | Uncharacterized protein | LPNLGRRMVGAW | 0.65 | 2 | 1.31 | 10.55 | 59 |
| F7QWM3 | Uncharacterized protein | LSHVNGGYNRLAGRIGHYT | 0.44 | 3 | 1.31 | 9.52 | 60 |
| W9BZA4 | Uncharacterized protein | IGGLLGGIFGLLRK | 0.65 | 2 | 1.30 | 9.13 | 61 |
| A0A0E4FN40 | Uncharacterized protein | RNGARIAGRLVGIA | 0.43 | 3 | 1.29 | 5.11 | 62 |
| G8UJS1 | Uncharacterized protein | RNGARIAGRLVGIA | 0.43 | 3 | 1.29 | 5.11 | 63 |
| R4LKG1 | Uncharacterized protein | ANHANGLFRRVL | 0.51 | 2.5 | 1.27 | 5.56 | 64 |
| A9EDI1 | Uncharacterized protein | FSKLQKLIGGVEK | 0.63 | 2 | 1.25 | 9.73 | 65 |
| D5BXP3 | Uncharacterized protein | AARRAFASGRKA | 0.31 | 4 | 1.25 | 10.83 | 66 |
| A0A081QIR4 | Uncharacterized protein | GIAGVLNAAVQIFNAGYKFGSDFARRGR | 0.41 | 3 | 1.24 | 4.99 | 67 |
| A0A0F3HMJ4 | Uncharacterized protein | GIAGVLNAAVQIFNAGYKFGSDFARRGR | 0.41 | 3 | 1.24 | 4.99 | 68 |
| G0IAJ8 | Uncharacterized protein | GIAGVLNAAVQIFNAGYKFGSDFARRGR | 0.41 | 3 | 1.24 | 4.99 | 69 |
| A0A086B184 | Uncharacterized protein | GKSCWKILKDLIGL | 0.62 | 2 | 1.24 | 9.6 | 70 |
| A0A0F2CT49 | Uncharacterized protein | GIAGVLNAAVQIFNAGYKFGSDLARRGR | 0.41 | 3 | 1.24 | 4.99 | 71 |
| A8SCT0 | Uncharacterized protein | IGKVFNYIARIFSGASSIIN | 0.62 | 2 | 1.23 | 4.94 | 72 |
| D4K7U6 | Uncharacterized protein | IGKVFNYIARIFSGASSIIN | 0.62 | 2 | 1.23 | 4.94 | 73 |
| Q040A1 | Uncharacterized protein | VAKDAWNHLDQIRSGWRKAGNS | 0.48 | 2.5 | 1.20 | 9.3 | 74 |
| A0A0A2CDF7 | Uncharacterized protein | KTYKKVAKKIEDYVDNGP | 0.58 | 2 | 1.16 | 4.81 | 75 |
| F8HC99 | Uncharacterized protein | FSSGYKFGTDLARRGR | 0.38 | 3 | 1.15 | 9.6 | 76 |
| F8LGE1 | Uncharacterized protein | FSSGYKFGTDLARRGR | 0.38 | 3 | 1.15 | 9.87 | 77 |
| T0TBH8 | Uncharacterized protein | TTLLTGVFGWLKKF | 0.57 | 2 | 1.13 | 6.54 | 78 |
| C7H4R6 | Uncharacterized protein | VGKLFNYIARVFSAGSSIVN | 0.54 | 2 | 1.09 | 4.66 | 79 |
| F8N9W8 | Uncharacterized protein | IDSFKKGADKANR | 0.54 | 2 | 1.09 | 4.83 | 80 |
| A0A077E9X6 | Uncharacterized protein | KKGLRQITGGME | 0.54 | 2 | 1.09 | 8.84 | 81 |
| E2ZM85 | Uncharacterized protein | NVGKVFNAIARIFSGASS | 0.54 | 2 | 1.07 | 4.99 | 82 |
| R4KBP7 | Uncharacterized protein | VKNLFNQISQVQQKTAQEFQQMMNKFPQ | 0.53 | 2 | 1.06 | 6.12 | 83 |
| U2J1M1 | Uncharacterized protein | GGNWINVFKSVVDIARRG | 0.52 | 2 | 1.05 | 9.4 | 84 |
| A0A0G0A6F6 | Uncharacterized protein | TSLESLGSRARRVLKEIS | 0.52 | 2 | 1.05 | 9.81 | 85 |
| R7F343 | Uncharacterized protein | GSLLNAFVDILKVLLDAGRSVGSGIRRAT | 0.52 | 2 | 1.04 | 9.1 | 86 |
| Q8RTK6 | Uncharacterized protein | KTLATVIGGGKGGGILSWFIGESSDIWKGFKKG | 0.23 | 4.5 | 1.03 | 9.7 | 87 |
| | | MNHYNK | | | | | |
| U2XMS3 | Uncharacterized protein | KTLATVIGGGKGGGILSWFIGESSDIWKGFKKG | 0.23 | 4.5 | 1.03 | 9.7 | 88 |
| | | MNHYNK | | | | | |
| R5MS15 | Uncharacterized protein | GSILNSFIRYVNVFFEIGQAFGSAIRR | 0.51 | 2 | 1.02 | 9.92 | 89 |
| D2JC35 | Uncharacterized protein | GEKIGKLLREVREAK | 0.51 | 2 | 1.02 | 9.22 | 90 |
| F7QWF6 | Uncharacterized protein | IVQGAVAVFKSGYRH | 0.41 | 2.5 | 1.01 | 9.9 | 91 |
| H3NCZ5 | Uncharacterized protein | TVGGSYSSACKRVWNAK | 0.34 | 3 | 1.01 | 10.12 | 92 |
| R5MUU1 | Uncharacterized protein | ATFFNALARSLSTISDIGRSLGTAIR | 0.50 | 2 | 1.01 | 10.44 | 93 |
| R6GW12 | Uncharacterized protein | TQMINSFVKLVSTLLDLGRTMGSALR | 0.50 | 2 | 0.99 | 10.01 | 94 |
| R6C487 | Uncharacterized protein | SVGTIINAVVKAISLLNELGQQLGSAIRR | 0.48 | 2 | 0.95 | 9.74 | 95 |
| A0A0F4NQ60 | Uncharacterized protein | SGPAWLSRYLHK | 0.38 | 2.5 | 0.95 | 5.28 | 96 |
| Q5FI70 | Uncharacterized protein | VGRGLWENLSNIFKH | 0.62 | 1.5 | 0.93 | 10.35 | 97 |
| X5CZ49 | Putative inducing | THLLAGLWDWFKR | 0.60 | 1.5 | 0.91 | 5.38 | 98 |
| | factor protein | | | | | | |
| R9JDF1 | Uncharacterized protein | SSVKQFFEKGARSA | 0.45 | 2 | 0.89 | 6.7 | 99 |
| E1TJ04 | Uncharacterized protein | AAHRAIAKVAAT | 0.36 | 2.5 | 0.89 | 10.89 | 100 |
| A7GJQ0 | Uncharacterized protein | TGFYNGYRNAGR | 0.44 | 2 | 0.88 | 5.5 | 101 |
| A0A0A5I2E5 | Uncharacterized protein | SGPAWLNRYLNK | 0.44 | 2 | 0.88 | 4.73 | 102 |
| A0A0H2MUQ5 | Uncharacterized protein | SGPAWLNRYLNK | 0.44 | 2 | 0.88 | 4.71 | 103 |
| F9T9J1 | Uncharacterized protein | SGPAWLNRYLNK | 0.44 | 2 | 0.88 | 4.71 | 104 |
| R6FA54 | Uncharacterized protein | KVKQFFEKGARSAME | 0.44 | 2 | 0.88 | 6.55 | 105 |
| R6W634 | Uncharacterized protein | KVKQFFEKGARSAME | 0.44 | 2 | 0.88 | 6.05 | 106 |
| R2P4R2 | Bacteriocin-type signal | NNLSKGGAKCGAAIAGGL | 0.44 | 2 | 0.87 | 9.39 | 107 |
| | sequence | | | | | | |
| A0A086B186 | Uncharacterized protein | KEILTHVKGGAAGSGYINTVSGECNSSGRSCWK | 0.34 | 2.5 | 0.86 | 9.7 | 108 |
| | | ALKDLLG | | | | | |
| U2I0Q7 | Uncharacterized protein | QGGGVIGRLIKAAANA | 0.43 | 2 | 0.86 | 5.1 | 109 |
| A0A0D6XRS2 | Uncharacterized protein | GWAGRIAGKVAGGVRSLADG | 0.43 | 2 | 0.86 | 5.13 | 110 |
| R9N5C7 | Uncharacterized protein | TQVKQFFEKGAKSA | 0.43 | 2 | 0.85 | 7.89 | 111 |
| U2QX19 | Uncharacterized protein | QQLVTLIRGGCAKIIA | 0.42 | 2 | 0.85 | 10.56 | 112 |
| G6F0H8 | Uncharacterized protein | ANIIRTGLKNGDNWATIGKNIG | 0.42 | 2 | 0.85 | 10.29 | 113 |
| R7IG68 | Uncharacterized protein | LDSINGGGKKLWYLLGAGLSFFLGLFSGLVNPV | 0.42 | 2 | 0.84 | 7.9 | 114 |
| | | KC | | | | | |
| R6SN80 | Uncharacterized protein | MKTCKGCGASILST | 0.42 | 2 | 0.84 | 8.75 | 115 |
| C9QIT0 | Uncharacterized protein | NGPAWLNRYLTK | 0.41 | 2 | 0.83 | 4.9 | 116 |
| A3HWV0 | Putative bacteriocin- | VSTARKLGRDFGDFLAYCAVVSIFIADAAKDAI | 0.41 | 2 | 0.83 | 7.87 | 117 |
| | type signal sequence | KTFTKL | | | | | |
| A0A0J1CUK6 | Uncharacterized protein | QALRSYAKPAGS | 0.41 | 2 | 0.81 | 10.67 | 118 |
| E7S5Z5 | Uncharacterized protein | LKSIKGGGDTVFDKVNHFF | 0.53 | 1.5 | 0.80 | 9.14 | 119 |
| A0A075SGV6 | Bacteriocin-type signal | LKSIKGGGDTVFDKVNHFF | 0.53 | 1.5 | 0.80 | 9.44 | 120 |
| | sequence | | | | | | |
| A0A0A2YHL6 | Uncharacterized protein | SISSGLTAGYSLVRR | 0.38 | 2 | 0.77 | 4.6 | 121 |
| U1I9V8 | Uncharacterized protein | SISSGLTAGYSLVRR | 0.38 | 2 | 0.77 | 4.6 | 122 |
| X8JLI8 | Bacteriocin-type signal | VINEFVKGFRQAW | 0.75 | 1 | 0.75 | 5.05 | 123 |
| | sequence | | | | | | |
| A0A074JK52 | Signal peptide protein | VINEFVKGFRQAW | 0.75 | 1 | 0.75 | 5.05 | 124 |
| E4S7K8 | Uncharacterized protein | LGKLYKWITNVIDNIF | 0.74 | 1 | 0.74 | 7.79 | 125 |
| G2SRZ9 | Putative bacteriocin | SVNRWGAAVGTGGKA | 0.37 | 2 | 0.74 | 9.59 | 126 |
| A0A077KSQ4 | Uncharacterized protein | KGGNKAITPVREG | 0.37 | 2 | 0.74 | 9.78 | 127 |
| A0A0C1TV13 | Uncharacterized protein | IYDGVKAAVNKLAHT | 0.49 | 1.5 | 0.74 | 6.03 | 128 |
| A0A073BCT9 | Uncharacterized protein | QRVHAACNELLRGQ | 0.48 | 1.5 | 0.72 | 7.73 | 129 |
| U2PR16 | Uncharacterized protein | GDSYLQKLTGIRRHRLRALFGGGEE | 0.29 | 2.5 | 0.72 | 6.53 | 130 |
| E3CPS1 | Bacteriocin-type signal | VINEFVKGFHHAW | 0.70 | 1 | 0.70 | 5.43 | 131 |
| | sequence | | | | | | |
| E8KT23 | Bacteriocin-type signal | VINEFVKGFHHAW | 0.70 | 1 | 0.70 | 5.43 | 132 |
| | sequence | | | | | | |
| I7KH06 | Uncharacterized protein | AGYTIGKDIAKR | 0.35 | 2 | 0.69 | 4.51 | 133 |
| A0A015XA39 | Uncharacterized protein | YNKMEKILEKLS | 0.69 | 1 | 0.69 | 4.77 | 134 |
| A0A015YJT2 | Uncharacterized protein | YNKMEKILEKLS | 0.69 | 1 | 0.69 | 4.77 | 135 |
| A0A015Z6Y8 | Uncharacterized protein | YNKMEKILEKLS | 0.69 | 1 | 0.69 | 4.77 | 136 |
| A0A016GFI9 | Uncharacterized protein | YNKMEKILEKLS | 0.69 | 1 | 0.69 | 4.77 | 137 |
| A0A017PDT5 | Uncharacterized protein | YNKMEKILEKLS | 0.69 | 1 | 0.69 | 4.77 | 138 |
| A0A0E2B796 | Uncharacterized protein | YNKMEKILEKLS | 0.69 | 1 | 0.69 | 4.77 | 139 |
| K1G3D7 | Uncharacterized protein | YNKMEKILEKLS | 0.69 | 1 | 0.69 | 4.49 | 140 |
| K1G6C4 | Uncharacterized protein | YNKMEKILEKLS | 0.69 | 1 | 0.69 | 4.49 | 141 |
| D6E338 | Uncharacterized protein | SIEKYVKNYLDFIKKAID | 0.69 | 1 | 0.69 | 5.18 | 142 |
| F4KYL7 | Uncharacterized protein | HKVADTYNAIAKT | 0.46 | 1.5 | 0.69 | 4.78 | 143 |
| U2DX84 | Bacteriocin-type signal | GGNIFKSIWNWIKE | 0.67 | 1 | 0.67 | 8.23 | 144 |
| | sequence | | | | | | |
| A0RKV6 | Uncharacterized protein | VIGIIAGGAAIIDYGNDFVKGAKKGAK | 0.34 | 2 | 0.67 | 9.4 | 145 |
| K9EK75 | Uncharacterized protein | ARGAKEAVDRIRI | 0.34 | 2 | 0.67 | 7.63 | 146 |
| A0A0D5BK00 | Uncharacterized protein | APGGCRSIAGAGPK | 0.33 | 2 | 0.67 | 8.83 | 147 |
| T0Q2K8 | Uncharacterized protein | APGGCRSIAGAGPK | 0.33 | 2 | 0.67 | 8.59 | 148 |
| T0V1T0 | Uncharacterized protein | SWANYLSRYLKE | 0.67 | 1 | 0.67 | 7.98 | 149 |
| G2JCE9 | Bacteriocin-type signal | LGKAYKWIANLIESIF | 0.67 | 1 | 0.67 | 7.95 | 150 |
| | sequence | | | | | | |
| A0A077EH26 | Uncharacterized protein | APGGCRSLAGAGPK | 0.33 | 2 | 0.66 | 8.59 | 151 |
| A0A098G366 | Uncharacterized protein | SGGNRVHEGFDRMR | 0.44 | 1.5 | 0.66 | 11.57 | 152 |
| S9P0Z7 | Uncharacterized protein | GGGRGGRDGRGGRGGRGGRGGRGGRGGY | 0.09 | 7 | 0.66 | 6.18 | 153 |
| D4BZB4 | Uncharacterized protein | NRLVQVVGGWLHSFAL | 0.43 | 1.5 | 0.65 | 9.3 | 154 |
| D4BZB5 | Uncharacterized protein | NRLVQVVGGWLHSFAL | 0.43 | 1.5 | 0.65 | 6.69 | 155 |
| A0A0A2CWR1 | Uncharacterized protein | YKKVANYLREID | 0.65 | 1 | 0.65 | 4.29 | 156 |
| B4U1M3 | Uncharacterized protein | KVVKWVAGFFE | 0.65 | 1 | 0.65 | 5.99 | 157 |
| A0A0D1BIJ1 | Contig000026, whole | KVVKWVAGFFE | 0.65 | 1 | 0.65 | 5.99 | 158 |
| | genome shotgun sequence | | | | | | |
| A0A0D0Z7R1 | Contig000027, whole | KVVKWVAGFFE | 0.65 | 1 | 0.65 | 5.99 | 159 |
| | genome shotgun sequence | | | | | | |
| A0A0D1AR79 | Contig000095, whole | KVVKWVAGFFE | 0.65 | 1 | 0.65 | 5.99 | 160 |
| | genome shotgun sequence | | | | | | |
| C0MDI1 | Putative competence | KVVKWVAGFFE | 0.65 | 1 | 0.65 | 5.99 | 161 |
| | stimulating peptide | | | | | | |
| A0A0G2Z858 | Uncharacterized protein | GGGWIDGIKKIINL | 0.62 | 1 | 0.62 | 4.94 | 162 |
| L0G0E1 | Uncharacterized protein | AGIGAALFSSLIVTGGGMMSVGYSCGNKVKNGL | 0.31 | 2 | 0.61 | 9.14 | 163 |
| | | RDY | | | | | |
| A0A0B8T3S9 | Uncharacterized protein | GYEVGKSAAERARR | 0.30 | 2 | 0.61 | 5.23 | 164 |
| D5T3D9 | Uncharacterized protein | GGDVVKTLTRLFNQ | 0.60 | 1 | 0.60 | 4.86 | 165 |
| A0A0H3J316 | Uncharacterized protein | GGGIYDKINKFLSN | 0.58 | 1 | 0.58 | 4.19 | 166 |
| J3CI08 | Uncharacterized protein | LRTVTGGLEDCINPATGGCRKI | 0.57 | 1 | 0.57 | 9.56 | 167 |
| C1CCS2 | Conserved domain | AALGCAAGGVKYGKILGPWGAAIGGIGGAV | 0.29 | 2 | 0.57 | 5.89 | 168 |
| | protein | | | | | | |
| F8N6B2 | Uncharacterized protein | LGRGLGCGRGCGGC | 0.29 | 2 | 0.57 | 4.6 | 169 |
| Q729Z0 | Uncharacterized protein | GGVLRRCWRDWLDSFFAVQ | 0.57 | 1 | 0.57 | 11.25 | 170 |
| G6J8U1 | Putative membrane | AALGCAAGGVKYGRLLGPWGAAIGGIGGAV | 0.28 | 2 | 0.57 | 5.89 | 171 |
| | protein | | | | | | |
| C2EJ80 | Bacteriocin-type signal | NFGKSYYGKCSF | 0.28 | 2 | 0.56 | 9.7 | 172 |
| | sequence | | | | | | |
| A0A062WNU4 | Uncharacterized protein | GYIGGGNHLCKG | 0.37 | 1.5 | 0.56 | 5.08 | 173 |
| A0A064BZ31 | Uncharacterized protein | GYIGGGNHLCKG | 0.37 | 1.5 | 0.56 | 5.5 | 174 |
| A0A081QCP4 | Uncharacterized protein | GYIGGGNHLCKG | 0.37 | 1.5 | 0.56 | 5.48 | 175 |
| A0A0B7LYG0 | Uncharacterized protein | GYIGGGNHLCKG | 0.37 | 1.5 | 0.56 | 5.5 | 176 |
| A0A0E9GYM9 | Uncharacterized protein | GYIGGGNHLCKG | 0.37 | 1.5 | 0.56 | 5.11 | 177 |
| A5LKF7 | Uncharacterized protein | GYIGGGNHLCKG | 0.37 | 1.5 | 0.56 | 5.5 | 178 |
| A5M755 | Uncharacterized protein | GYIGGGNHLCKG | 0.37 | 1.5 | 0.56 | 5.5 | 179 |
| C1C6Q6 | Uncharacterized protein | GYIGGGNHLCKG | 0.37 | 1.5 | 0.56 | 5.5 | 180 |
| C1CDS2 | Uncharacterized protein | GYIGGGNHLCKG | 0.37 | 1.5 | 0.56 | 5.5 | 181 |
| D6ZLM5 | Uncharacterized protein | GYIGGGNHLCKG | 0.37 | 1.5 | 0.56 | 5.5 | 182 |
| E0Q149 | Uncharacterized protein | GYIGGGNHLCKG | 0.37 | 1.5 | 0.56 | 5.48 | 183 |
| G6LM33 | Uncharacterized protein | GYIGGGNHLCKG | 0.37 | 1.5 | 0.56 | 5.5 | 184 |
| M5K5U3 | Uncharacterized protein | GYIGGGNHLCKG | 0.37 | 1.5 | 0.56 | 5.5 | 185 |
| M5K8K8 | Uncharacterized protein | GYIGGGNHLCKG | 0.37 | 1.5 | 0.56 | 5.5 | 186 |
| M5N786 | Uncharacterized protein | GYIGGGNHLCKG | 0.37 | 1.5 | 0.56 | 5.5 | 187 |
| Q8CYW4 | Uncharacterized protein | GYIGGGNHLCKG | 0.37 | 1.5 | 0.56 | 5.5 | 188 |
| S9RF90 | Uncharacterized protein | GYIGGGNHLCKG | 0.37 | 1.5 | 0.56 | 7.79 | 189 |
| V8IBA1 | Uncharacterized protein | GYIGGGNHLCKG | 0.37 | 1.5 | 0.56 | 5.48 | 190 |
| V8ID01 | Uncharacterized protein | GYIGGGNHLCKG | 0.37 | 1.5 | 0.56 | 5.48 | 191 |
| A0A0F7HB86 | Uncharacterized protein | GTSAVTVCRRVATC | 0.28 | 2 | 0.56 | 8.86 | 192 |
| U2NQ09 | Uncharacterized protein | GTSAVTVCRRVATC | 0.28 | 2 | 0.56 | 8.86 | 193 |
| B9DTX9 | Putative bacteriocin | GGLGGLQTGIKYCKV | 0.27 | 2 | 0.55 | 9.1 | 194 |
| A0A0D0Z195 | Contig000004, whole | GAIGCASRGVKLGSRIGPWGAVIGGVGSAA | 0.18 | 3 | 0.54 | 7.71 | 195 |
| | genome shotgun sequence | | | | | | |
| A0A0D1AE73 | Contig000013, whole | GAIGCASRGVKLGSRIGPWGAVIGGVGSAA | 0.18 | 3 | 0.54 | 7.71 | 196 |
| | genome shotgun sequence | | | | | | |
| A0A0D0ZZF1 | Contig000024, whole | GAIGCASRGVKLGSRIGPWGAVIGGVGSAA | 0.18 | 3 | 0.54 | 7.71 | 197 |
| | genome shotgun sequence | | | | | | |
| C0MH64 | Putative bacteriocin | GAIGCASRGVKLGSRIGPWGAVIGGVGSAA | 0.18 | 3 | 0.54 | 7.71 | 198 |
| C0CJI1 | Uncharacterized protein | MEKVSGGASRYQH | 0.36 | 1.5 | 0.54 | 9.99 | 199 |
| R5C531 | Uncharacterized protein | MEKVSGGASRYQH | 0.36 | 1.5 | 0.54 | 9.99 | 200 |
| R6VS52 | Uncharacterized protein | IDGYCSKCAEKIRNGQ | 0.54 | 1 | 0.54 | 8.37 | 201 |
| C1C5R3 | Conserved domain | AALGCAAGGVKYGRLLGLWGAAIGGIGGAV | 0.27 | 2 | 0.54 | 5.89 | 202 |
| | protein | | | | | | |
| A0A085HBT8 | Uncharacterized protein | TTLSQFFSKLFG | 0.54 | 1 | 0.54 | 4.39 | 203 |
| C5RBL9 | Uncharacterized protein | VKSLLGLLGGIMGM | 0.53 | 1 | 0.53 | 9.99 | 204 |
| M1WT49 | Uncharacterized protein | EILKGGKTLVTPL | 0.53 | 1 | 0.53 | 9.96 | 205 |
| W0BBY2 | Uncharacterized protein | LSKVSGGASQMTH | 0.35 | 1.5 | 0.52 | 4.45 | 206 |
| Q5FI68 | Uncharacterized protein | GSGRGGGAQMRAIGS | 0.26 | 2 | 0.52 | 11.62 | 207 |
| R6R6T2 | Uncharacterized protein | TLFNYIARVFSAGSSIINN | 0.52 | 1 | 0.52 | 5.14 | 208 |
| A0A0H2UPL5 | Uncharacterized protein | SGYIGGGNHLCKG | 0.35 | 1.5 | 0.52 | 5.5 | 209 |
| K9RTP0 | Uncharacterized protein | QLTGGFRSYGRQLDG | 0.52 | 1 | 0.52 | 4.61 | 210 |
| R2M4F0 | Uncharacterized protein | TAKQCLQAIGSWG | 0.52 | 1 | 0.52 | 5.98 | 211 |
| R7ICH1 | Uncharacterized protein | ASFFNSVARILRTAYDIG | 0.52 | 1 | 0.52 | 9.85 | 212 |
| R7HKI2 | Uncharacterized protein | SMLTAIYKTLEVIYQIGESLGNYIRR | 0.51 | 1 | 0.51 | 6.15 | 213 |
| Q38Y63 | Hypothetical small | GLGKLVRAGVDIG | 0.51 | 1 | 0.51 | 9.99 | 214 |
| | peptide | | | | | | |
| Q6KCF2 | Putative beta-peptide | GLGKLVRAGVDIG | 0.51 | 1 | 0.51 | 9.99 | 215 |
| W4QHA2 | Uncharacterized protein | LLGLCTGFYGVYKTV | 0.50 | 1 | 0.50 | 9.65 | 216 |
| H2AM33 | Uncharacterized protein | AGLGNGLKQWNT | 0.49 | 1 | 0.49 | 6.53 | 217 |
| H1WT90 | Uncharacterized protein | VVSFGKGIVSAFG | 0.49 | 1 | 0.49 | 9.87 | 218 |
| R6N077 | Uncharacterized protein | ATMMNAIYKTIEIIFNIGEAFGSYIRR | 0.49 | 1 | 0.49 | 6.15 | 219 |
| A0A086AYC6 | Uncharacterized protein | GECRKYGRGCAEQ | 0.48 | 1 | 0.48 | 9.3 | 220 |
| R7M280 | Uncharacterized protein | MINALTKAVTALYDLGKGFGSA | 0.48 | 1 | 0.48 | 8.98 | 221 |
| R5HCU1 | Uncharacterized protein | ASFLNAISRGIETFLNLGRM | 0.48 | 1 | 0.48 | 9.78 | 222 |
| F4AFZ5 | Uncharacterized protein | VLSAASGAGTGIKACKSFG | 0.24 | 2 | 0.48 | 9.14 | 223 |
| W4QQL4 | Uncharacterized protein | LFGLATGFYGVYRAI | 0.48 | 1 | 0.48 | 9.87 | 224 |
| I9T9Y4 | Uncharacterized protein | YEKINGVLTRVFTFS | 0.47 | 1 | 0.47 | 7.9 | 225 |
| A0A0F3H1F2 | Uncharacterized protein | ATVNTVVNGWMRYG | 0.47 | 1 | 0.47 | 9.18 | 226 |
| T0TP63 | Uncharacterized protein | ATVNTVVNGWMRYG | 0.47 | 1 | 0.47 | 9.18 | 227 |
| E3CS41 | Uncharacterized protein | ATVNTVVNGWMKYG | 0.47 | 1 | 0.47 | 9.14 | 228 |
| J8IIA4 | Uncharacterized protein | RIVTALSAFFTSGFTVL | 0.47 | 1 | 0.47 | 9.63 | 229 |
| A0A0F2DG91 | Uncharacterized protein | MGQGALNSYRDAWK | 0.46 | 1 | 0.46 | 4.61 | 230 |
| E1LEJ5 | Uncharacterized protein | MGQGALNSYRDAWK | 0.46 | 1 | 0.46 | 4.61 | 231 |
| C0BVX0 | Uncharacterized protein | EVKKLFQDAANSAMK | 0.46 | 1 | 0.46 | 6.03 | 232 |
| T1ZF04 | Uncharacterized protein | AKSGIAGGAGNGLRLG | 0.23 | 2 | 0.46 | 8.77 | 233 |
| L0KBX6 | Uncharacterized protein | KAMVKDGWKLIETA | 0.46 | 1 | 0.46 | 4.07 | 234 |
| A0A0D5BQU9 | Uncharacterized protein | GKLRAWGGGCDS | 0.45 | 1 | 0.45 | 9.34 | 235 |
| F9HJW5 | Uncharacterized protein | WGVFKSVIGTFGP | 0.45 | 1 | 0.45 | 4.96 | 236 |
| A0A086AWR6 | Uncharacterized protein | CANGVCRPIAGAG | 0.45 | 1 | 0.45 | 8.86 | 237 |
| Q8FUD5 | Uncharacterized protein | GGGGIARELATMLRP | 0.44 | 1 | 0.44 | 11.6 | 238 |
| I0GCL8 | Uncharacterized protein | HDGLKVAEAIIKAITH | 0.44 | 1 | 0.44 | 5.13 | 239 |
| C2LRN7 | Uncharacterized protein | NGGGILSSVAGLVKDTWGTLYSTGRDFGRSVVN | 0.43 | 1 | 0.43 | 4.44 | 240 |
| E0S535 | Uncharacterized protein | KYYTAICATYQAALDLCARVG | 0.42 | 1 | 0.42 | 6.08 | 241 |
| A0A077EAB3 | Uncharacterized protein | MAQLKGGIKSVEG | 0.42 | 1 | 0.42 | 9.05 | 242 |
| R7P6R1 | Uncharacterized protein | IIGAISGYINPVKC | 0.41 | 1 | 0.41 | 9.04 | 243 |
| K2DVP9 | Uncharacterized protein | KTADQVAREAGAAGREIK | 0.41 | 1 | 0.41 | 9.05 | 244 |
| A0A0F2E6T0 | Excreted peptide | STICTRMTGYGNGII | 0.41 | 1 | 0.41 | 3.62 | 245 |
| S9SM70 | Excreted peptide | STICTRMTGYGNGII | 0.41 | 1 | 0.41 | 3.62 | 246 |
| A0A023BZ22 | Uncharacterized protein | VLAAIGGLYKAGEAIGKG | 0.41 | 1 | 0.41 | 5.05 | 247 |
| A0A077ECN9 | Uncharacterized protein | MAQLKGGMQEIAKTCAAGS | 0.40 | 1 | 0.40 | 9.07 | 248 |
| A0A077EE44 | Uncharacterized protein | MAQLKGGMQEIAKTCAAGS | 0.40 | 1 | 0.40 | 9.07 | 249 |
| U2CMK8 | Uncharacterized protein | KVKQFFEQGAKSAME | 0.40 | 1 | 0.40 | 6.04 | 250 |
| F8LL37 | Uncharacterized | VATGVGNGLRLGIKTRTWQGAVAGAAGGAIVGG | 0.13 | 3 | 0.40 | 6.01 | 251 |
| | conserved protein | VGY | | | | | |
| F8HFT9 | Pore-forming peptide, | VATGVGNGLRLGIKTRTWQGAVAGAAGGAIVGG | 0.13 | 3 | 0.40 | 6.01 | 252 |
| | putative bacteriocin | VGY | | | | | |
| F8LRT1 | Pore-forming peptide, | VATGVGNGLRLGIKTRTWQGAVAGAAGGAIVGG | 0.13 | 3 | 0.40 | 6.01 | 253 |
| | putative bacteriocin | VGY | | | | | |
| Q5M2W2 | Pore-forming peptide, | VATGVGNGLRLGIKTRTWQGAVAGAAGGAIVGG | 0.13 | 3 | 0.40 | 6.01 | 254 |
| | putative bacteriocin | VGY | | | | | |
| A0A086B2P4 | Uncharacterized protein | LKTIKGGGDQGV | 0.39 | 1 | 0.39 | 9.27 | 255 |
| M4YVZ4 | Hypothetical membrane | IIGGLGGLASGLKF | 0.38 | 1 | 0.38 | 5.46 | 256 |
| | associated protein | | | | | | |
| A0A0E2IVJ2 | Uncharacterized protein | IAGGAGNGLRLGIKTRTWQGVVAGAVGGAIIGG | 0.13 | 3 | 0.38 | 8.77 | 257 |
| | | VGY | | | | | |
| I0S9I1 | Uncharacterized protein | IAGGAGNGLRLGIKTRTWQGVVAGAVGGAIIGG | 0.13 | 3 | 0.38 | 8.77 | 258 |
| | | VGY | | | | | |
| A0A087QC98 | Uncharacterized protein | GGKAVNGFVNGN | 0.37 | 1 | 0.37 | 6.17 | 259 |
| D1YGH0 | Uncharacterized protein | GGKAVNGFVNGN | 0.37 | 1 | 0.37 | 6.17 | 260 |
| A0A077EA21 | Uncharacterized protein | MAQLKGGIKNADG | 0.37 | 1 | 0.37 | 9.05 | 261 |
| A0A077EFY9 | Uncharacterized protein | MAQLKGGIKNADG | 0.37 | 1 | 0.37 | 9.05 | 262 |
| A0A086A6U3 | Uncharacterized protein | RACTLISSGCAQ | 0.37 | 1 | 0.37 | 8.59 | 263 |
| F8NCK2 | Uncharacterized protein | QKGMSQVRGGVQE | 0.37 | 1 | 0.37 | 5.28 | 264 |
| A0A0F2CQG4 | Uncharacterized protein | SIWKVGGAVVGGATALFA | 0.37 | 1 | 0.37 | 4.2 | 265 |
| A0A0H0YQB5 | Uncharacterized protein | GLGAIARSWAQGGF | 0.35 | 1 | 0.35 | 5.04 | 266 |
| A0A0H0YSB4 | Uncharacterized protein | GLGAIARSWAQGGF | 0.35 | 1 | 0.35 | 5 | 267 |
| K9E1K0 | Uncharacterized protein | CAKSGNAAGAAVMSAH | 0.23 | 1.5 | 0.35 | 8.43 | 268 |
| J3CSM9 | Uncharacterized protein | VRALLGGIIATGSA | 0.35 | 1 | 0.35 | 11.65 | 269 |
| A0A0H2UTD9 | Uncharacterized protein | VIGGLGGLASGLKF | 0.34 | 1 | 0.34 | 6.5 | 270 |
| Q9A137 | Uncharacterized protein | VIGGLGGLASGLKF | 0.34 | 1 | 0.34 | 6.5 | 271 |
| C5WF58 | Hypothetical membrane | VIGGLGGLASGLKF | 0.34 | 1 | 0.34 | 6.5 | 272 |
| | associated protein | | | | | | |
| Q1J801 | Hypothetical membrane | VIGGLGGLASGLKF | 0.34 | 1 | 0.34 | 6.5 | 273 |
| | associated protein | | | | | | |
| Q1JI59 | Hypothetical membrane | VIGGLGGLASGLKF | 0.34 | 1 | 0.34 | 6.5 | 274 |
| | associated protein | | | | | | |
| Q1JN16 | Hypothetical membrane | VIGGLGGLASGLKF | 0.34 | 1 | 0.34 | 6.5 | 275 |
| | associated protein | | | | | | |
| Q48UV8 | Hypothetical membrane | VIGGLGGLASGLKF | 0.34 | 1 | 0.34 | 6.5 | 276 |
| | associated protein | | | | | | |
| Q5XDF5 | Hypothetical membrane | VIGGLGGLASGLKF | 0.34 | 1 | 0.34 | 6.5 | 277 |
| | associated protein | | | | | | |
| I7J3B0 | Uncharacterized protein | DFWHKLMDSLCRNFNS | 0.68 | 0.5 | 0.34 | 6.54 | 278 |
| F5ZAJ3 | Uncharacterized protein | RGGGWVGAAIGAISGGLG | 0.34 | 1 | 0.34 | 4.4 | 279 |
| G0IAJ6 | Uncharacterized protein | SIWKIGAAVAGGAAALFA | 0.34 | 1 | 0.34 | 4.2 | 280 |
| K0ZN36 | Uncharacterized protein | AAKIAGGITAAG | 0.34 | 1 | 0.34 | 4.08 | 281 |
| K1A1E3 | Uncharacterized protein | AAKIAGGITAAG | 0.34 | 1 | 0.34 | 4.08 | 282 |
| A0A0F3HMF2 | Uncharacterized protein | SIWKVGAAVAGGATALFA | 0.33 | 1 | 0.33 | 4.25 | 283 |
| X8FLG5 | Uncharacterized protein | AAGWKACSSIYGM | 0.33 | 1 | 0.33 | 5.49 | 284 |
| A0A0H8RN19 | Putative transporter | LVSGGIALGSRCG | 0.32 | 1 | 0.32 | 4.89 | 285 |
| A0A0J7IN65 | Uncharacterized protein | TGPKYCSTYIGC | 0.32 | 1 | 0.32 | 9.04 | 286 |
| S6C1D9 | Uncharacterized protein | GLGAIARSWAQGGFA | 0.32 | 1 | 0.32 | 5.04 | 287 |
| A5M594 | Uncharacterized protein | KAIAGGTALIGSGWAAG | 0.31 | 1 | 0.31 | 3.57 | 288 |
| U2EXV9 | Uncharacterized protein | TFGGGGDAAAAGVRK | 0.31 | 1 | 0.31 | 7.89 | 289 |
| A5LJ32 | Uncharacterized protein | KVIAGGTALIGSGLAAG | 0.30 | 1 | 0.30 | 3.57 | 290 |
| A0A0B7LKX0 | Excreted peptide | KVIAGGTALIGSGLAAG | 0.30 | 1 | 0.30 | 3.57 | 291 |
| Q6SZ94 | Bacteriocin-type signal | NSAANLATGGAAGWKS | 0.29 | 1 | 0.29 | 9.7 | 292 |
| | sequence-containing | | | | | | |
| | protein (MundKS) | | | | | | |
| | (Mundticin) | | | | | | |
| A0RJ68 | Uncharacterized protein | AGVAGFTKVAGAITAGAG | 0.29 | 1 | 0.29 | 9.52 | 293 |
| B3Z892 | Uncharacterized protein | AGVAGFTKVAGAITAGAG | 0.29 | 1 | 0.29 | 9.52 | 294 |
| C2UP09 | Uncharacterized protein | AGVAGFTKVAGAITAGAG | 0.29 | 1 | 0.29 | 9.7 | 295 |
| A0A0H2UN03 | Uncharacterized protein | KAIAGGTALIGSGLAAG | 0.29 | 1 | 0.29 | 3.57 | 296 |
| A0A0H2ZQ92 | Uncharacterized protein | KAIAGGTALIGSGLAAG | 0.29 | 1 | 0.29 | 3.57 | 297 |
| B1I840 | Uncharacterized protein | KAIAGGTALIGSGLAAG | 0.29 | 1 | 0.29 | 3.57 | 298 |
| B2IRL0 | Uncharacterized protein | KAIAGGTALIGSGLAAG | 0.29 | 1 | 0.29 | 3.57 | 299 |
| C1CAF3 | Uncharacterized protein | KAIAGGTALIGSGLAAG | 0.29 | 1 | 0.29 | 3.57 | 300 |
| C1CBU7 | Uncharacterized protein | KAIAGGTALIGSGLAAG | 0.29 | 1 | 0.29 | 3.57 | 301 |
| G0IBD6 | Uncharacterized protein | KAIAGGTALIGSGLAAG | 0.29 | 1 | 0.29 | 3.57 | 302 |
| J1DKQ8 | Uncharacterized protein | KAIAGGTALIGSGLAAG | 0.29 | 1 | 0.29 | 3.57 | 303 |
| Q8CZA0 | Uncharacterized protein | KAIAGGTALIGSGLAAG | 0.29 | 1 | 0.29 | 3.57 | 304 |
| RON8A8 | Uncharacterized protein | KAIAGGTALIGSGLAAG | 0.29 | 1 | 0.29 | 3.57 | 305 |
| A0A0C1QXM7 | Uncharacterized protein | VLKSMNAVASAFQFIAL | 0.28 | 1 | 0.28 | 4.13 | 306 |
| A0A069P4B0 | Uncharacterized protein | AGGRAVSAIVDGR | 0.28 | 1 | 0.28 | 5.03 | 307 |
| B1BHK0 | Uncharacterized protein | KAVALISAGVTTLAGAAA | 0.28 | 1 | 0.28 | 4.78 | 308 |
| B1BU21 | Uncharacterized protein | KAVALISAGVTTLAGAAA | 0.28 | 1 | 0.28 | 4.78 | 309 |
| B1RC11 | Uncharacterized protein | KAVALISAGVTTLAGAAA | 0.28 | 1 | 0.28 | 4.78 | 310 |
| I7H0S7 | Uncharacterized protein | KAVALISAGVTTLAGAAA | 0.28 | 1 | 0.28 | 4.78 | 311 |
| A0A0D7QJF4 | Uncharacterized protein | LAGKALDALSHATDHTPTNVLNGMLGAIGR | 0.28 | 1 | 0.28 | 4.97 | 312 |
| R7AVY5 | Uncharacterized protein | MKEYAQSASDAKVKQFFEKGAKSA | 0.14 | 2 | 0.27 | 7.88 | 313 |
| A0A0D1B9N5 | Contig000008, whole | SHLPSIFEKFCNWIGM | 0.54 | 0.5 | 0.27 | 9.89 | 314 |
| | genome shotgun sequence | | | | | | |
| B1BHJ9 | Uncharacterized protein | TGAAIAKGAAAIGTA | 0.26 | 1 | 0.26 | 6.53 | 315 |
| B1BU20 | Uncharacterized protein | TGAAIAKGAAAIGTA | 0.26 | 1 | 0.26 | 6.53 | 316 |
| B1RC12 | Uncharacterized protein | TGAAIAKGAAAIGTA | 0.26 | 1 | 0.26 | 6.53 | 317 |
| I7H937 | Putative bacteriocin | TGAAIAKGAAAIGTA | 0.26 | 1 | 0.26 | 6.53 | 318 |
| E3CPS2 | Uncharacterized protein | HTVYDFGRGFVDGFRG | 0.51 | 0.5 | 0.26 | 6.51 | 319 |
| E8KT24 | Uncharacterized protein | HTVYDFGRGFVDGFRG | 0.51 | 0.5 | 0.26 | 6.51 | 320 |
| T0TQ78 | Uncharacterized protein | HTVYDFGRGFVDGFRG | 0.51 | 0.5 | 0.26 | 8.12 | 321 |
| X8JP08 | Uncharacterized protein | HTVYDFGRGFVDGFRG | 0.51 | 0.5 | 0.26 | 8.12 | 322 |
| E1M7J2 | Uncharacterized protein | KAIAGGTALVGSGLTAG | 0.26 | 1 | 0.26 | 3.71 | 323 |
| D6E340 | Uncharacterized protein | FWTLVAAGFAGGIATGVSR | 0.25 | 1 | 0.25 | 8.14 | 324 |
| A0A0F2CYI7 | Uncharacterized protein | KAIAGGTALVGSGLAAG | 0.25 | 1 | 0.25 | 3.66 | 325 |
| A0A0F2DGV3 | Uncharacterized protein | KAIAGGTALVGSGLAAG | 0.25 | 1 | 0.25 | 3.5 | 326 |
| A0A0F3HMX5 | Uncharacterized protein | KAIAGGTALVGSGLAAG | 0.25 | 1 | 0.25 | 3.71 | 327 |

Class II bacteriocins are typically small, cationic peptides that often contain a conserved leader peptide sequence that is important for downstream processing of the mature peptide. This leader peptide is characterized as having a highly conserved double-glycine motif that has been shown to be essential for proper cleavage of the bacteriocin precursor. Beyond this motif, a number of other residues within the leader sequence are also conserved to varying degrees.

Within the field, it has been assumed that the conservation of these residues was likely due to a specificity of the enzyme that carried out cleavage of the signal peptide domain. This supposition has recently been borne out with the structural characterization of a prototypic ABC transporter, demonstrating that only very small residues, such as glycine, can be accommodated in the cleft containing the active site of this protease. This example also suggested that the enzyme would have strong preferences for specific polar or non-polar residues along the signal peptide template.

In the present study, an alignment of more than 200 prototypic class II bacteriocins was carried out to generate a more relaxed signal peptide consensus sequence. In particular, in addition to the C-terminal double glycine motif, this consensus allowed for any polar residue at positions-9 and -11 of the signal peptide backbone. Moreover, an expanded set of hydrophobic residues was allowed at positions-4 and -7, to generate a consensus that may be more representative of a broad number of known bacteriocin sequences.

Once complete, this expanded consensus was used as an initial step within a multi-component search method to probe for new bacteriocin sequences in genomic data templates. This retrieved a large percentage (88-90%) of the currently known class IIa and IIb linear bacteriocins. Hence, the application of this multi-component search method may provide a useful way to rapidly screen new sequence data for the presence of bacteriocin sequences. Such methods may serve as an adjunct to currently used computational gene identification tools as many small peptides are not correctly identified or annotated in current genome sequencing projects.

Beyond the signal peptide consensus screen, a second component of the search method made use of a newly identified amphipathic search formula to probe for α-helical domains within the retrieved peptide dataset. The tests using this formula have demonstrated that it is highly accurate, identifying alpha-helical domains with greater than 90% efficiency. Application of the amphipathic search formula returned virtually all of the bacteriocins identified in the primary signal peptide-based screen with the exception of the pediocin peptides.

### Residue Frequency

### Abundance of Glycine and Alanine

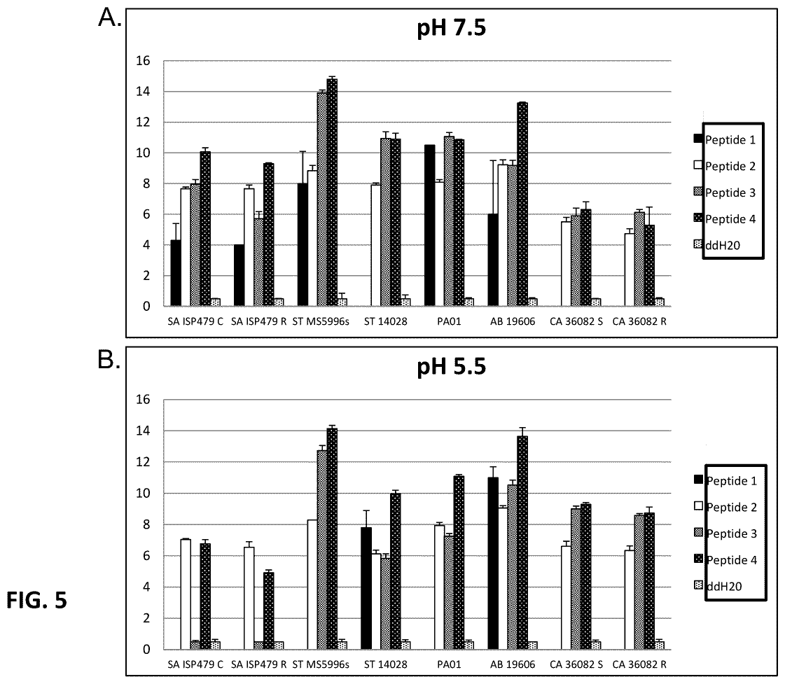

As the amphipathic search formula returns aligned datasets, it was possible to determine the frequency of individual residues on both the polar and non-polar faces of a predicted 18 residue α-helical span of returned peptides. One outcome of this analysis was the finding that glycine was the most abundant residue at nearly all positions of the polar face of the peptide and, if not the most abundant, then highly represented on the non-polar face of the returned sequences (FIG. 5). It is also of interest that after glycine one of the second most abundant residues at all positions on both peptide faces was alanine.

These findings lend support to one of the reigning theories regarding the mechanism by which many α-helical antimicrobial peptides are presumed to limit self-toxicity prior to interacting with their intended target. This theory proposes that an abundance of small, sterically-unrestrained residues with a high degree of rotational freedom, such as glycine and alanine, serve to keep α-helical antimicrobial peptides in an unstructured and non-toxic conformation in aqueous environments. Such peptides only then become organized to adopt their microbicidal amphipathic structure when they encounter the hydrophobic milieu of the target membrane. Support for this theory is provided by: 1) the finding of an abundance of glycine, and to a lesser extent alanine, residues in the α-helical antimicrobial peptides of many organisms; and 2) a significant number of structural studies which demonstrate that many α-helical antimicrobial peptides are unstructured in aqueous solutions only to adopt an α-helical conformation in the presence of membrane mimetic environments.

In the current example, the finding of an abundance of glycine and alanine in the retrieved bacteriocins would suggest that these peptides may also utilize a similar mechanism to limit self-toxicity.

### Preference of Lysine Over Arginine

One additional mechanism by which α-helical antimicrobial peptides are thought to exert their microbicidal effect, is via a relative abundance of cationic residues which are thought to enhance their selectivity towards anionic surface lipids and the increased electronegative potential of many bacterial species. In the present study, a large proportion of the retrieved sequences were cationic in nature, an observation that supports this hypothesis. One additional finding was a strong preference for an abundance of lysine over arginine in the retrieved bacteriocin sequences. While the significance of this observation is not known, it is in alignment with a similar preference for lysine over arginine in many α-helical antimicrobial peptides of eukaryotes. Biophysical studies have found that lysine is less efficient at generating negative Gaussian membrane curvature, and pore-like structures, than arginine. However, many α-helical antimicrobial peptides have an increased net hydrophobicity, as compared with other more arginine-rich classes of antimicrobial proteins, a feature that may compensate for this reduced permeabilizing efficiency.

### Spectrum/Potency

Sequences retrieved by the multi-component sequence formula were screened for a number of biophysical parameters, and based on these findings, and their localization to bacteriocin-like operons, four putative bacteriocin peptides were synthesized and assessed for microbicidal activity. Notably, all four peptides were found to have activity against a broad spectrum of microorganisms, with particular potency against a number of Gram negative pathogens. While historically it was reported that bacteriocin peptides were relatively narrow spectrum antibiotics, being most potent against closely related Gram positive organisms, more recent studies have found that in fact many bacteriocins have a much broader spectrum with microbicidal activity towards Gram negative and fungal organisms as well. These more recent studies reflect what was found in this report where all four putative bacteriocin peptides had activity against Gram positive, Gram negative and fungal organisms.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. An isolated peptide that is not longer than 45 amino acid residues in length and comprises an amino acid sequence of formula [LI]-[KREDNQSTYH]-X-[KREDNQSTYH]-X-[ML V]-X-X-[IVLT]-X-G-G, wherein X denotes any amino acid, wherein the amino acid sequence is selected from the group consisting of SEQ ID NO:1-327.

2. The isolated peptide of claim 1, wherein the peptide has antimicrobial activity.

3. The isolated peptide of claim 1, wherein the peptide is not longer than 40 amino acid residues in length.

4. The isolated peptide of claim 3, wherein the peptide is not longer than 35 amino acid residues in length.

5. A method of treating an infection in a patient in need thereof, comprising administering to the patient an effective amount of the peptide of claim 1.

6. The method of claim 5, wherein the infection is caused by a Gram-negative bacterium, a Gram-positive bacterium or a fungus.


SEQUENCE LISTING (1884 sequences)

SEQ ID NO: 1 - protein, 15 aa, synthetic construct
EWGKQLGKWIGSKIK

SEQ ID NO: 2 - protein, 22 aa, synthetic construct
KYVKNYLDFIKKAIDIFRPMPI

SEQ ID NO: 3 - protein, 15 aa, synthetic construct
KWAKSAGKWIASKIK

SEQ ID NO: 4 - protein, 21 aa, synthetic construct
KIGKTVGTIVRKGFEIWSIFK

SEQ ID NO: 5 - protein, 28 aa, synthetic construct
FKVIVTDAGHYPREWGKQLGKWIGSKIK

SEQ ID NO: 6 - protein, 29 aa, synthetic construct
KRNYSIEKYVKNYLDFIKKAIDIFRPMPI

SEQ ID NO: 7 - protein, 27 aa, synthetic construct
KTIATNATYYPNKWAKSAGKWIASKIK

SEQ ID NO: 8 - protein, 28 aa, synthetic construct
QYDKTGYKIGKTVGTIVRKGFEIWSIFK

SEQ ID NO: 9 - protein, 17 aa, synthetic construct
RQMVRHLRRFMRRYGRC

SEQ ID NO: 10 - protein, 14 aa, synthetic construct
KNGLKKFFKWVRKL

SEQ ID NO: 11 - protein, 18 aa, synthetic construct
SLKKALKKAVSGLGKIIK

SEQ ID NO: 12 - protein, 13 aa, synthetic construct
KTLTKFFKSLIKR

SEQ ID NO: 13 - protein, 12 aa, synthetic construct
RCRKVYHRLWGK

SEQ ID NO: 14 - protein, 15 aa, synthetic construct
SLRKALKKIIDAIVK

SEQ ID NO: 15 - protein, 13 aa, synthetic construct
SQLYKNLFKVFRK

SEQ ID NO: 16 - protein, 19 aa, synthetic construct
LQMGATAIKQVKKLFKKWG

SEQ ID NO: 17 - protein, 15 aa, synthetic construct
YKNVKAFIEKCRKVY

SEQ ID NO: 18 - protein, 13 aa, synthetic construct
REYLNKITKWIKH

SEQ ID NO: 19 - protein, 13 aa, synthetic construct
WIGKVGKQYKKAT

SEQ ID NO: 20 - protein, 31 aa, synthetic construct
GSIISSIVRGGILILELGRSFGSALRRLLKK

SEQ ID NO: 21 - protein, 15 aa, synthetic construct
RQAAKAIGKAVGKLF

SEQ ID NO: 22 - protein, 22 aa, synthetic construct
MRNGFEIFKKIAKSIIDAFRHA

SEQ ID NO: 23 - protein, 21 aa, synthetic construct
AVGKNVWEFGKGFVKGWNKAK

SEQ ID NO: 24 - protein, 22 aa, synthetic construct
KTGYKIGKTVGTIVRKGFEIWS

SEQ ID NO: 25 - protein, 12 aa, synthetic construct
GGSKGWNKFWKS

SEQ ID NO: 26 - protein, 12 aa, synthetic construct
GGSKGWNKFWKS

SEQ ID NO: 27 - protein, 16 aa, synthetic construct
GGGAWKNFWSSLRKGF

SEQ ID NO: 28 - protein, 14 aa, synthetic construct
KQWYRLGERVGRVA

SEQ ID NO: 29 - protein, 13 aa, synthetic construct
GSACAYMRRVCKK

SEQ ID NO: 30 - protein, 13 aa, synthetic construct
RRITSFFANLFQK

SEQ ID NO: 31 - protein, 13 aa, synthetic construct
QKAMKNIRGGARG

SEQ ID NO: 32 - protein, 15 aa, synthetic construct
VKWCNKLFGTGRHVA

SEQ ID NO: 33 - protein, 29 aa, synthetic construct
LSHISGGVTRYRHHEKKSWIDDFMKGFKK

SEQ ID NO: 34 - protein, 12 aa, synthetic construct
SITNFWKKYFNH

SEQ ID NO: 35 - protein, 12 aa, synthetic construct
SITNFWKKYFNH

SEQ ID NO: 36 - protein, 15 aa, synthetic construct
YNPFKNIFHRFSNGH

SEQ ID NO: 37 - protein, 13 aa, synthetic construct
YKNVKAFIEKCRS

SEQ ID NO: 38 - protein, 18 aa, synthetic construct
GRFFGGIYNAGRSFGRNV

SEQ ID NO: 39 - protein, 18 aa, synthetic construct
GRFFGGIYNAGRSFGRNV

SEQ ID NO: 40 - protein, 12 aa, synthetic construct
RGIIGIGKKLFG

SEQ ID NO: 41 - protein, 29 aa, synthetic construct
GTILNQLNKLISILVDSGKSLGSSIRRIS

SEQ ID NO: 42 - protein, 28 aa, synthetic construct
INGGKTIATNATYYPNKWAKSVGKWIAS

SEQ ID NO: 43 - protein, 13 aa, synthetic construct
SNFIHKIKQIFTH

SEQ ID NO: 44 - protein, 13 aa, synthetic construct
IGGSAKSYIRRLG

SEQ ID NO: 45 - protein, 31 aa, synthetic construct
GSLFSALAKGFNVFVDMGRALGSSIRRLVNN

SEQ ID NO: 46 - protein, 29 aa, synthetic construct
LSHISGGVTRYRYHEKKSWIDDFMKGFKK

SEQ ID NO: 47 - protein, 29 aa, synthetic construct
LSHISGGVTRYRYHEKKSWIDDFMKGFKK

SEQ ID NO: 48 - protein, 29 aa, synthetic construct
AAFINGVKGIFSIFFDFGNSLGSSIRRIK

SEQ ID NO: 49 - protein, 12 aa, synthetic construct
RGMIGIGKKLFG

SEQ ID NO: 50 - protein, 29 aa, synthetic construct
ATFINAIVKGLSLIIELGKSLGSSFRRIT

SEQ ID NO: 51 - protein, 28 aa, synthetic construct
INGGKTIATNATYYPNKWAKSAGKWIAS

SEQ ID NO: 52 - protein, 27 aa, synthetic construct
QIYKEAKDFVNNRAGNLIRGFKDVWKN

SEQ ID NO: 53 - protein, 13 aa, synthetic construct
LSRVKGGMSRIAG

SEQ ID NO: 54 - protein, 12 aa, synthetic construct
KWGKELGRWIGS

SEQ ID NO: 55 - protein, 13 aa, synthetic construct
REWGKQLGKWIGS

SEQ ID NO: 56 - protein, 16 aa, synthetic construct
SGGGKFIGNLIKLARP

SEQ ID NO: 57 - protein, 13 aa, synthetic construct
LRFLDRIMGGFRQ

SEQ ID NO: 58 - protein, 12 aa, synthetic construct
FGGLLRHLFGFK

SEQ ID NO: 59 - protein, 12 aa, synthetic construct
LPNLGRRMVGAW

SEQ ID NO: 60 - protein, 19 aa, synthetic construct
LSHVNGGYNRLAGRIGHYT

SEQ ID NO: 61 - protein, 14 aa, synthetic construct
IGGLLGGIFGLLRK

SEQ ID NO: 62 - protein, 14 aa, synthetic construct
RNGARIAGRLVGIA

SEQ ID NO: 63 - protein, 14 aa, synthetic construct
RNGARIAGRLVGIA

SEQ ID NO: 64 - protein, 12 aa, synthetic construct
ANHANGLFRRVL

SEQ ID NO: 65 - protein, 13 aa, synthetic construct
FSKLQKLIGGVEK

SEQ ID NO: 66 - protein, 12 aa, synthetic construct
AARRAFASGRKA

SEQ ID NO: 67 - protein, 28 aa, synthetic construct
GIAGVLNAAVQIFNAGYKFGSDFARRGR

SEQ ID NO: 68 - protein, 28 aa, synthetic construct
GIAGVLNAAVQIFNAGYKFGSDFARRGR

SEQ ID NO: 69 - protein, 28 aa, synthetic construct
GIAGVLNAAVQIFNAGYKFGSDFARRGR

SEQ ID NO: 70 - protein, 14 aa, synthetic construct
GKSCWKILKDLIGL

SEQ ID NO: 71 - protein, 28 aa, synthetic construct
GIAGVLNAAVQIFNAGYKFGSDLARRGR

SEQ ID NO: 72 - protein, 20 aa, synthetic construct
IGKVFNYIARIFSGASSIIN

SEQ ID NO: 73 - protein, 20 aa, synthetic construct
IGKVFNYIARIFSGASSIIN

SEQ ID NO: 74 - protein, 22 aa, synthetic construct
VAKDAWNHLDQIRSGWRKAGNS

SEQ ID NO: 75 - protein, 18 aa, synthetic construct
KTYKKVAKKIEDYVDNGP

SEQ ID NO: 76 - protein, 16 aa, synthetic construct
FSSGYKFGTDLARRGR

SEQ ID NO: 77 - protein, 16 aa, synthetic construct
FSSGYKFGTDLARRGR

SEQ ID NO: 78 - protein, 14 aa, synthetic construct
TTLLTGVFGWLKKF

SEQ ID NO: 79 - protein, 20 aa, synthetic construct
VGKLFNYIARVFSAGSSIVN

SEQ ID NO: 80 - protein, 13 aa, synthetic construct
IDSFKKGADKANR

SEQ ID NO: 81 - protein, 12 aa, synthetic construct
KKGLRQITGGME

SEQ ID NO: 82 - protein, 18 aa, synthetic construct
NVGKVFNAIARIFSGASS

SEQ ID NO: 83 - protein, 28 aa, synthetic construct
VKNLFNQISQVQQKTAQEFQQMMNKFPQ

SEQ ID NO: 84 - protein, 18 aa, synthetic construct
GGNWINVFKSVVDIARRG

SEQ ID NO: 85 - protein, 18 aa, synthetic construct
TSLESLGSRARRVLKEIS

SEQ ID NO: 86 - protein, 29 aa, synthetic construct
GSLLNAFVDILKVLLDAGRSVGSGIRRAT

SEQ ID NO: 87 - protein, 39 aa, synthetic construct
KTLATVIGGGKGGGILSWFIGESSDIWKGFKKGMNHYNK

SEQ ID NO: 88 - protein, 39 aa, synthetic construct; identical to SEQ ID NO: 87

SEQ ID NO: 89 - protein, 27 aa, synthetic construct
GSILNSFIRYVNVFFEIGQAFGSAIRR

SEQ ID NO: 90 - protein, 15 aa, synthetic construct
GEKIGKLLREVREAK

SEQ ID NO: 91 - protein, 15 aa, synthetic construct
IVQGAVAVFKSGYRH

SEQ ID NO: 92 - protein, 17 aa, synthetic construct
TVGGSYSSACKRVWNAK

SEQ ID NO: 93 - protein, 26 aa, synthetic construct
ATFFNALARSLSTISDIGRSLGTAIR

SEQ ID NO: 94 - protein, 26 aa, synthetic construct
TQMINSFVKLVSTLLDLGRTMGSALR

SEQ ID NO: 95 - protein, 29 aa, synthetic construct
SVGTIINAVVKAISLLNELGQQLGSAIRR

SEQ ID NO: 96 - protein, 12 aa, synthetic construct
SGPAWLSRYLHK

SEQ ID NO: 97 - protein, 15 aa, synthetic construct
VGRGLWENLSNIFKH

SEQ ID NO: 98 - protein, 13 aa, synthetic construct
THLLAGLWDWFKR

SEQ ID NO: 99 - protein, 14 aa, synthetic construct
SSVKQFFEKGARSA

SEQ ID NO: 100 - protein, 12 aa, synthetic construct
AAHRAIAKVAAT

SEQ ID NO: 101 - protein, 12 aa, synthetic construct
TGFYNGYRNAGR

SEQ ID NO: 102 - protein, 12 aa, synthetic construct
SGPAWLNRYLNK

SEQ ID NO: 103 - protein, 12 aa, synthetic construct
SGPAWLNRYLNK

SEQ ID NO: 104 - protein, 12 aa, synthetic construct
SGPAWLNRYLNK

SEQ ID NO: 105 - protein, 15 aa, synthetic construct
KVKQFFEKGARSAME

SEQ ID NO: 106 - protein, 15 aa, synthetic construct
KVKQFFEKGARSAME

SEQ ID NO: 107 - protein, 18 aa, synthetic construct
NNLSKGGAKCGAAIAGGL

SEQ ID NO: 108 - protein, 40 aa, synthetic construct
KEILTHVKGGAAGSGYINTVSGECNSSGRSCWKALKDLLG

SEQ ID NO: 109 - protein, 16 aa, synthetic construct
QGGGVIGRLIKAAANA

SEQ ID NO: 110 - protein, 20 aa, synthetic construct
GWAGRIAGKVAGGVRSLADG

SEQ ID NO: 111 - protein, 14 aa, synthetic construct
TQVKQFFEKGAKSA

SEQ ID NO: 112 - protein, 16 aa, synthetic construct
QQLVTLIRGGCAKIIA

SEQ ID NO: 113 - protein, 22 aa, synthetic construct
ANIIRTGLKNGDNWATIGKNIG

SEQ ID NO: 114 - protein, 35 aa, synthetic construct
LDSINGGGKKLWYLLGAGLSFFLGLFSGLVNPVKC

SEQ ID NO: 115 - protein, 14 aa, synthetic construct
MKTCKGCGASILST

SEQ ID NO: 116 - protein, 12 aa, synthetic construct
NGPAWLNRYLTK

SEQ ID NO: 117 - protein, 39 aa, synthetic construct
VSTARKLGRDFGDFLAYCAVVSIFIADAAKDAIKTFTKL

SEQ ID NO: 118 - protein, 12 aa, synthetic construct
QALRSYAKPAGS

SEQ ID NO: 119 - protein, 19 aa, synthetic construct
LKSIKGGGDTVFDKVNHFF

SEQ ID NO: 120 - protein, 19 aa, synthetic construct
LKSIKGGGDTVFDKVNHFF

SEQ ID NO: 121 - protein, 15 aa, synthetic construct
SISSGLTAGYSLVRR

SEQ ID NO: 122 - protein, 15 aa, synthetic construct
SISSGLTAGYSLVRR

SEQ ID NO: 123 - protein, 13 aa, synthetic construct
VINEFVKGFRQAW

SEQ ID NO: 124 - protein, 13 aa, synthetic construct
VINEFVKGFRQAW

SEQ ID NO: 125 - protein, 16 aa, synthetic construct
LGKLYKWITNVIDNIF

SEQ ID NO: 126 - protein, 15 aa, synthetic construct
SVNRWGAAVGTGGKA

SEQ ID NO: 127 - protein, 13 aa, synthetic construct
KGGNKAITPVREG

SEQ ID NO: 128 - protein, 15 aa, synthetic construct
IYDGVKAAVNKLAHT

SEQ ID NO: 129 - protein, 14 aa, synthetic construct
QRVHAACNELLRGQ

SEQ ID NO: 130 - protein, 25 aa, synthetic construct
GDSYLQKLTGIRRHRLRALFGGGEE

SEQ ID NO: 131 - protein, 13 aa, synthetic construct
VINEFVKGFHHAW

SEQ ID NO: 132 - protein, 13 aa, synthetic construct
VINEFVKGFHHAW

SEQ ID NO: 133 - protein, 12 aa, synthetic construct
AGYTIGKDIAKR

SEQ ID NO: 134 - protein, 12 aa, synthetic construct
YNKMEKILEKLS

SEQ ID NO: 135 - protein, 12 aa, synthetic construct
YNKMEKILEKLS

SEQ ID NO: 136 - protein, 12 aa, synthetic construct
YNKMEKILEKLS

SEQ ID NO: 137 - protein, 12 aa, synthetic construct
YNKMEKILEKLS

SEQ ID NO: 138 - protein, 12 aa, synthetic construct
YNKMEKILEKLS

SEQ ID NO: 139 - protein, 12 aa, synthetic construct
YNKMEKILEKLS

SEQ ID NO: 140 - protein, 12 aa, synthetic construct
YNKMEKILEKLS

SEQ ID NO: 141 - protein, 12 aa, synthetic construct
YNKMEKILEKLS

SEQ ID NO: 142 - protein, 18 aa, synthetic construct
SIEKYVKNYLDFIKKAID

SEQ ID NO: 143 - protein, 13 aa, synthetic construct
HKVADTYNAIAKT

SEQ ID NO: 144 - protein, 14 aa, synthetic construct
GGNIFKSIWNWIKE

SEQ ID NO: 145 - protein, 27 aa, synthetic construct
VIGIIAGGAAIIDYGNDFVKGAKKGAK

SEQ ID NO: 146 - protein, 13 aa, synthetic construct
ARGAKEAVDRIRI

SEQ ID NO: 147 - protein, 14 aa, synthetic construct
APGGCRSIAGAGPK

SEQ ID NO: 148 - protein, 14 aa, synthetic construct
APGGCRSIAGAGPK

SEQ ID NO: 149 - protein, 12 aa, synthetic construct
SWANYLSRYLKE

SEQ ID NO: 150 - protein, 16 aa, synthetic construct
LGKAYKWIANLIESIF

SEQ ID NO: 151 - protein, 14 aa, synthetic construct
APGGCRSLAGAGPK

SEQ ID NO: 152 - protein, 14 aa, synthetic construct
SGGNRVHEGFDRMR

SEQ ID NO: 153 - protein, 28 aa, synthetic construct
GGGRGGRDGRGGRGGRGGRGGRGGRGGY

SEQ ID NO: 154 - protein, 16 aa, synthetic construct
NRLVQVVGGWLHSFAL

SEQ ID NO: 155 - protein, 16 aa, synthetic construct
NRLVQVVGGWLHSFAL

SEQ ID NO: 156 - protein, 12 aa, synthetic construct
YKKVANYLREID

SEQ ID NO: 157 - protein, 11 aa, synthetic construct
KVVKWVAGFFE

SEQ ID NO: 158 - protein, 11 aa, synthetic construct
KVVKWVAGFFE

SEQ ID NO: 159 - protein, 11 aa, synthetic construct
KVVKWVAGFFE

SEQ ID NO: 160 - protein, 11 aa, synthetic construct
KVVKWVAGFFE

SEQ ID NO: 161 - protein, 11 aa, synthetic construct
KVVKWVAGFFE

SEQ ID NO: 162 - protein, 14 aa, synthetic construct
GGGWIDGIKKIINL

SEQ ID NO: 163 - protein, 36 aa, synthetic construct
AGIGAALFSSLIVTGGGMMSVGYSCGNKVKNGLRDY

SEQ ID NO: 164 - protein, 14 aa, synthetic construct
GYEVGKSAAERARR

SEQ ID NO: 165 - protein, 14 aa, synthetic construct
GGDVVKTLTRLFNQ

SEQ ID NO: 166 - protein, 14 aa, synthetic construct
GGGIYDKINKFLSN

SEQ ID NO: 167 - protein, 22 aa, synthetic construct
LRTVTGGLEDCINPATGGCRKI

SEQ ID NO: 168 - protein, 30 aa, synthetic construct
AALGCAAGGVKYGKILGPWGAAIGGIGGAV

SEQ ID NO: 169 - protein, 14 aa, synthetic construct
LGRGLGCGRGCGGC

SEQ ID NO: 170 - protein, 19 aa, synthetic construct
GGVLRRCWRDWLDSFFAVQ

SEQ ID NO: 171 - protein, 30 aa, synthetic construct
AALGCAAGGVKYGRLLGPWGAAIGGIGGAV

SEQ ID NO: 172 - protein, 12 aa, synthetic construct
NFGKSYYGKCSF

SEQ ID NO: 173 - protein, 12 aa, synthetic construct
GYIGGGNHLCKG

SEQ ID NO: 174 - protein, 12 aa, synthetic construct
GYIGGGNHLCKG

SEQ ID NO: 175 - protein, 12 aa, synthetic construct
GYIGGGNHLCKG

SEQ ID NO: 176 - protein, 12 aa, synthetic construct
GYIGGGNHLCKG

SEQ ID NO: 177 - protein, 12 aa, synthetic construct
GYIGGGNHLCKG

SEQ ID NO: 178 - protein, 12 aa, synthetic construct
GYIGGGNHLCKG

SEQ ID NO: 179 - protein, 12 aa, synthetic construct
GYIGGGNHLCKG

SEQ ID NO: 180 - protein, 12 aa, synthetic construct
GYIGGGNHLCKG

SEQ ID NO: 181 - protein, 12 aa, synthetic construct
GYIGGGNHLCKG

SEQ ID NO: 182 - protein, 12 aa, synthetic construct
GYIGGGNHLCKG

SEQ ID NO: 183 - protein, 12 aa, synthetic construct
GYIGGGNHLCKG

SEQ ID NO: 184 - protein, 12 aa, synthetic construct
GYIGGGNHLCKG

SEQ ID NO: 185 - protein, 12 aa, synthetic construct
GYIGGGNHLCKG

SEQ ID NO: 186 - protein, 12 aa, synthetic construct
GYIGGGNHLCKG

SEQ ID NO: 187 - protein, 12 aa, synthetic construct
GYIGGGNHLCKG

SEQ ID NO: 188 - protein, 12 aa, synthetic construct
GYIGGGNHLCKG

SEQ ID NO: 189 - protein, 12 aa, synthetic construct
GYIGGGNHLCKG

SEQ ID NO: 190 - protein, 12 aa, synthetic construct
GYIGGGNHLCKG

SEQ ID NO: 191 - protein, 12 aa, synthetic construct
GYIGGGNHLCKG

SEQ ID NO: 192 - protein, 14 aa, synthetic construct
GTSAVTVCRRVATC

SEQ ID NO: 193 - protein, 14 aa, synthetic construct
GTSAVTVCRRVATC

SEQ ID NO: 194 - protein, 15 aa, synthetic construct
GGLGGLQTGIKYCKV

SEQ ID NO: 195 - protein, 30 aa, synthetic construct
GAIGCASRGVKLGSRIGPWGAVIGGVGSAA

SEQ ID NO: 196 - protein, 30 aa, synthetic construct; identical to SEQ ID NO: 195

SEQ ID NO: 197 - protein, 30 aa, synthetic construct; identical to SEQ ID NO: 195

SEQ ID NO: 198 - protein, 30 aa, synthetic construct; identical to SEQ ID NO: 195

SEQ ID NO: 199 - protein, 13 aa, synthetic construct
MEKVSGGASRYQH

SEQ ID NO: 200 - protein, 13 aa, synthetic construct
MEKVSGGASRYQH

SEQ ID NO: 201 - protein, 16 aa, synthetic construct
IDGYCSKCAEKIRNGQ

SEQ ID NO: 202 - protein, 30 aa, synthetic construct
AALGCAAGGVKYGRLLGLWGAAIGGIGGAV

SEQ ID NO: 203 - protein, 12 aa, synthetic construct
TTLSQFFSKLFG

SEQ ID NO: 204 - protein, 14 aa, synthetic construct
VKSLLGLLGGIMGM

SEQ ID NO: 205 - protein, 13 aa, synthetic construct
EILKGGKTLVTPL

SEQ ID NO: 206 - protein, 13 aa, synthetic construct
LSKVSGGASQMTH

SEQ ID NO: 207 - protein, 15 aa, synthetic construct
GSGRGGGAQMRAIGS

SEQ ID NO: 208 - protein, 19 aa, synthetic construct
TLFNYIARVFSAGSSIINN

SEQ ID NO: 209 - protein, 13 aa, synthetic construct
SGYIGGGNHLCKG

SEQ ID NO: 210 - protein, 15 aa, synthetic construct
QLTGGFRSYGRQLDG

SEQ ID NO: 211 - protein, 13 aa, synthetic construct
TAKQCLQAIGSWG

SEQ ID NO: 212 - protein, 18 aa, synthetic construct
ASFFNSVARILRTAYDIG

SEQ ID NO: 213 - protein, 26 aa, synthetic construct
SMLTAIYKTLEVIYQIGESLGNYIRR

SEQ ID NO: 214 - protein, 13 aa, synthetic construct
GLGKLVRAGVDIG

SEQ ID NO: 215 - protein, 13 aa, synthetic construct
GLGKLVRAGVDIG

SEQ ID NO: 216 - protein, 15 aa, synthetic construct
LLGLCTGFYGVYKTV

SEQ ID NO: 217 - protein, 12 aa, synthetic construct
AGLGNGLKQWNT

SEQ ID NO: 218 - protein, 13 aa, synthetic construct
VVSFGKGIVSAFG

SEQ ID NO: 219 - protein, 27 aa, synthetic construct
ATMMNAIYKTIEIIFNIGEAFGSYIRR

SEQ ID NO: 220 - protein, 13 aa, synthetic construct
GECRKYGRGCAEQ

SEQ ID NO: 221 - protein, 22 aa, synthetic construct
MINALTKAVTALYDLGKGFGSA

SEQ ID NO: 222 - protein, 20 aa, synthetic construct
ASFLNAISRGIETFLNLGRM

SEQ ID NO: 223 - protein, 19 aa, synthetic construct
VLSAASGAGTGIKACKSFG

SEQ ID NO: 224 - protein, 15 aa, synthetic construct
LFGLATGFYGVYRAI

SEQ ID NO: 225 - protein, 15 aa, synthetic construct
YEKINGVLTRVFTFS

SEQ ID NO: 226 - protein, 14 aa, synthetic construct
ATVNTVVNGWMRYG

SEQ ID NO: 227 - protein, 14 aa, synthetic construct
ATVNTVVNGWMRYG

SEQ ID NO: 228 - protein, 14 aa, synthetic construct
ATVNTVVNGWMKYG

SEQ ID NO: 229 - protein, 17 aa, synthetic construct
RIVTALSAFFTSGFTVL

SEQ ID NO: 230 - protein, 14 aa, synthetic construct
MGQGALNSYRDAWK

SEQ ID NO: 231 - protein, 14 aa, synthetic construct
MGQGALNSYRDAWK

SEQ ID NO: 232 - protein, 15 aa, synthetic construct
EVKKLFQDAANSAMK

SEQ ID NO: 233 - protein, 16 aa, synthetic construct
AKSGIAGGAGNGLRLG

SEQ ID NO: 234 - protein, 14 aa, synthetic construct
KAMVKDGWKLIETA

SEQ ID NO: 235 - protein, 12 aa, synthetic construct
GKLRAWGGGCDS

SEQ ID NO: 236 - protein, 13 aa, synthetic construct
WGVFKSVIGTFGP

SEQ ID NO: 237 - protein, 13 aa, synthetic construct
CANGVCRPIAGAG

SEQ ID NO: 238 - protein, 15 aa, synthetic construct
GGGGIARELATMLRP

SEQ ID NO: 239 - protein, 16 aa, synthetic construct
HDGLKVAEAIIKAITH

SEQ ID NO: 240 - protein, 33 aa, synthetic construct
NGGGILSSVAGLVKDTWGTLYSTGRDFGRSVVN

SEQ ID NO: 241 - protein, 21 aa, synthetic construct
KYYTAICATYQAALDLCARVG

SEQ ID NO: 242 - protein, 13 aa, synthetic construct
MAQLKGGIKSVEG

SEQ ID NO: 243 - protein, 14 aa, synthetic construct
IIGAISGYINPVKC

SEQ ID NO: 244 - protein, 18 aa, synthetic construct
KTADQVAREAGAAGREIK

SEQ ID NO: 245 - protein, 15 aa, synthetic construct
STICTRMTGYGNGII

SEQ ID NO: 246 - protein, 15 aa, synthetic construct
STICTRMTGYGNGII

SEQ ID NO: 247 - protein, 18 aa, synthetic construct
VLAAIGGLYKAGEAIGKG

SEQ ID NO: 248 - protein, 19 aa, synthetic construct
MAQLKGGMQEIAKTCAAGS

SEQ ID NO: 249 - protein, 19 aa, synthetic construct
MAQLKGGMQEIAKTCAAGS

SEQ ID NO: 250 - protein, 15 aa, synthetic construct
KVKQFFEQGAKSAME

SEQ ID NO: 251 - protein, 36 aa, synthetic construct
VATGVGNGLRLGIKTRTWQGAVAGAAGGAIVGGVGY

SEQ ID NO: 252 - protein, 36 aa, synthetic construct; identical to SEQ ID NO: 251

SEQ ID NO: 253 - protein, 36 aa, synthetic construct; identical to SEQ ID NO: 251

SEQ ID NO: 254 - protein, 36 aa, synthetic construct; identical to SEQ ID NO: 251

SEQ ID NO: 255 - protein, 12 aa, synthetic construct
LKTIKGGGDQGV

SEQ ID NO: 256 - protein, 14 aa, synthetic construct
IIGGLGGLASGLKF

SEQ ID NO: 257 - protein, 36 aa, synthetic construct
IAGGAGNGLRLGIKTRTWQGVVAGAVGGAIIGGVGY

SEQ ID NO: 258 - protein, 36 aa, synthetic construct; identical to SEQ ID NO: 257

SEQ ID NO: 259 - protein, 12 aa, synthetic construct
GGKAVNGFVNGN

SEQ ID NO: 260 - protein, 12 aa, synthetic construct
GGKAVNGFVNGN

SEQ ID NO: 261 - protein, 13 aa, synthetic construct
MAQLKGGIKNADG

SEQ ID NO: 262 - protein, 13 aa, synthetic construct
MAQLKGGIKNADG

SEQ ID NO: 263 - protein, 12 aa, synthetic construct
RACTLISSGCAQ

SEQ ID NO: 264 - protein, 13 aa, synthetic construct
QKGMSQVRGGVQE

SEQ ID NO: 265 - protein, 18 aa, synthetic construct
SIWKVGGAVVGGATALFA

SEQ ID NO: 266 - protein, 14 aa, synthetic construct
GLGAIARSWAQGGF

SEQ ID NO: 267 - protein, 14 aa, synthetic construct
GLGAIARSWAQGGF

SEQ ID NO: 268 - protein, 16 aa, synthetic construct
CAKSGNAAGAAVMSAH

SEQ ID NO: 269 - protein, 14 aa, synthetic construct
VRALLGGIIATGSA

SEQ ID NO: 270 - protein, 14 aa, synthetic construct
VIGGLGGLASGLKF

SEQ ID NO: 271 - protein, 14 aa, synthetic construct
VIGGLGGLASGLKF

SEQ ID NO: 272 - protein, 14 aa, synthetic construct
VIGGLGGLASGLKF

SEQ ID NO: 273 - protein, 14 aa, synthetic construct
VIGGLGGLASGLKF

SEQ ID NO: 274 - protein, 14 aa, synthetic construct
VIGGLGGLASGLKF

SEQ ID NO: 275 - protein, 14 aa, synthetic construct
VIGGLGGLASGLKF

SEQ ID NO: 276 - protein, 14 aa, synthetic construct
VIGGLGGLASGLKF

SEQ ID NO: 277 - protein, 14 aa, synthetic construct
VIGGLGGLASGLKF

SEQ ID NO: 278 - protein, 16 aa, synthetic construct
DFWHKLMDSLCRNFNS

SEQ ID NO: 279 - protein, 18 aa, synthetic construct
RGGGWVGAAIGAISGGLG

SEQ ID NO: 280 - protein, 18 aa, synthetic construct
SIWKIGAAVAGGAAALFA

SEQ ID NO: 281 - protein, 12 aa, synthetic construct
AAKIAGGITAAG

SEQ ID NO: 282 - protein, 12 aa, synthetic construct
AAKIAGGITAAG

SEQ ID NO: 283 - protein, 18 aa, synthetic construct
SIWKVGAAVAGGATALFA

SEQ ID NO: 284 - protein, 13 aa, synthetic construct
AAGWKACSSIYGM

SEQ ID NO: 285 - protein, 13 aa, synthetic construct
LVSGGIALGSRCG

SEQ ID NO: 286 - protein, 12 aa, synthetic construct
TGPKYCSTYIGC

SEQ ID NO: 287 - protein, 15 aa, synthetic construct
GLGAIARSWAQGGFA

SEQ ID NO: 288 - protein, 17 aa, synthetic construct
KAIAGGTALIGSGWAAG

SEQ ID NO: 289 - protein, 15 aa, synthetic construct
TFGGGGDAAAAGVRK

SEQ ID NO: 290 - protein, 17 aa, synthetic construct
KVIAGGTALIGSGLAAG

SEQ ID NO: 291 - protein, 17 aa, synthetic construct
KVIAGGTALIGSGLAAG

SEQ ID NO: 292 - protein, 16 aa, synthetic construct
NSAANLATGGAAGWKS

SEQ ID NO: 293 - protein, 18 aa, synthetic construct
AGVAGFTKVAGAITAGAG

SEQ ID NO: 294 - protein, 18 aa, synthetic construct
AGVAGFTKVAGAITAGAG

SEQ ID NO: 295 - protein, 18 aa, synthetic construct
AGVAGFTKVAGAITAGAG

SEQ ID NO: 296 - protein, 17 aa, synthetic construct
KAIAGGTALIGSGLAAG

SEQ ID NO: 297 - protein, 17 aa, synthetic construct
KAIAGGTALIGSGLAAG

SEQ ID NO: 298 - protein, 17 aa, synthetic construct
KAIAGGTALIGSGLAAG

SEQ ID NO: 299 - protein, 17 aa, synthetic construct
KAIAGGTALIGSGLAAG

SEQ ID NO: 300 - protein, 17 aa, synthetic construct
KAIAGGTALIGSGLAAG

SEQ ID NO: 301 - protein, 17 aa, synthetic construct
KAIAGGTALIGSGLAAG

SEQ ID NO: 302 - protein, 17 aa, synthetic construct
KAIAGGTALIGSGLAAG

SEQ ID NO: 303 - protein, 17 aa, synthetic construct
KAIAGGTALIGSGLAAG

SEQ ID NO: 304 - protein, 17 aa, synthetic construct
KAIAGGTALIGSGLAAG

SEQ ID NO: 305 - protein, 17 aa, synthetic construct
KAIAGGTALIGSGLAAG

SEQ ID NO: 306 - protein, 17 aa, synthetic construct
VLKSMNAVASAFQFIAL

SEQ ID NO: 307 - protein, 13 aa, synthetic construct
AGGRAVSAIVDGR

SEQ ID NO: 308 - protein, 18 aa, synthetic construct
KAVALISAGVTTLAGAAA

SEQ ID NO: 309 - protein, 18 aa, synthetic construct
KAVALISAGVTTLAGAAA

SEQ ID NO: 310 - protein, 18 aa, synthetic construct
KAVALISAGVTTLAGAAA

SEQ ID NO: 311 - protein, 18 aa, synthetic construct
KAVALISAGVTTLAGAAA

SEQ ID NO: 312 - protein, 30 aa, synthetic construct
LAGKALDALSHATDHTPTNVLNGMLGAIGR

SEQ ID NO: 313 - protein, 24 aa, synthetic construct
MKEYAQSASDAKVKQFFEKGAKSA

SEQ ID NO: 314 - protein, 16 aa, synthetic construct
SHLPSIFEKFCNWIGM

SEQ ID NO: 315 - protein, 15 aa, synthetic construct
TGAAIAKGAAAIGTA

SEQ ID NO: 316 - protein, 15 aa, synthetic construct
TGAAIAKGAAAIGTA

SEQ ID NO: 317 - protein, 15 aa, synthetic construct
TGAAIAKGAAAIGTA

SEQ ID NO: 318 - protein, 15 aa, synthetic construct
TGAAIAKGAAAIGTA

SEQ ID NO: 319 - protein, 16 aa, synthetic construct
HTVYDFGRGFVDGFRG

SEQ ID NO: 320 - protein, 16 aa, synthetic construct
HTVYDFGRGFVDGFRG

SEQ ID NO: 321 - protein, 16 aa, synthetic construct
HTVYDFGRGFVDGFRG

SEQ ID NO: 322 - protein, 16 aa, synthetic construct
HTVYDFGRGFVDGFRG

SEQ ID NO: 323 - protein, 17 aa, synthetic construct
KAIAGGTALVGSGLTAG

SEQ ID NO: 324 - protein, 19 aa, synthetic construct
FWTLVAAGFAGGIATGVSR

SEQ ID NO: 325 - protein, 17 aa, synthetic construct
KAIAGGTALVGSGLAAG

SEQ ID NO: 326 - protein, 17 aa, synthetic construct
KAIAGGTALVGSGLAAG

SEQ ID NO: 327 - protein, 17 aa, synthetic construct
KAIAGGTALVGSGLAAG

SEQ ID NO: 328 - protein, 27 aa, synthetic construct
TGGGGGASLGTGRGQRATIDKPIHIRR

SEQ ID NO: 329 - protein, 27 aa, synthetic construct
TGGGGGASLGTGRGQRATIDKPIHIRR

SEQ ID NO: 330 - protein, 27 aa, synthetic construct
TGGGGCASLGTGRGQRATIDKPIHIRR

SEQ ID NO: 331 - protein, 39 aa, synthetic construct
QKAMKNIRGGARGTMGAGARARTSVRPLRTSLRRLFPNK

SEQ ID NO: 332 - protein, 40 aa, synthetic construct
ALLGGIIATGSARLSHARPQRGQTRDDRELDQRVRECGEW

SEQ ID NO: 333 - protein, 23 aa, synthetic construct
GRGGGAQMRAIGSIPPWRPNWWK

SEQ ID NO: 334 - protein, 30 aa, synthetic construct
GGGGIARELATMLRPFGATSLAVTRTGAPH

SEQ ID NO: 335 - protein, 30 aa, synthetic construct
SGGNRVHEGFDRMRPRDPQTIIDTLRNRRT

SEQ ID NO: 336 - protein, 39 aa, synthetic construct
RFLDRIMGGFRQNNISYRWLARWEKDIVTKEHISSHGIF

SEQ ID NO: 337 - protein, 22 aa, synthetic construct
DGVGKVVSGGSTVVDQFRRRSS

SEQ ID NO: 338 - protein, 38 aa, synthetic construct
SGGNRGTNAGQEIRESIERSAPAMRELLERFHNRNRSS

SEQ ID NO: 339 - protein, 19 aa, synthetic construct
GGVLRRCWRDWLDSFFAVQ

SEQ ID NO: 340 - protein, 18 aa, synthetic construct
VRGAVRTVAHLVTVEEVD

SEQ ID NO: 341 - protein, 31 aa, synthetic construct
YHIVRGALDTAGVDGRMQGRSKYGAKRPKKK

SEQ ID NO: 342 - protein, 18 aa, synthetic construct
AAHRAIAKVAATRHKRRS

SEQ ID NO: 343 - protein, 19 aa, synthetic construct
RKAHALLDAGARYTWPPRS

SEQ ID NO: 344 - protein, 12 aa, synthetic construct
AARRAFASGRKA

SEQ ID NO: 345 - protein, 15 aa, synthetic construct
YNPFKNIFHRFSNGH

SEQ ID NO: 346 - protein, 33 aa, synthetic construct
DEADGVGSRLDAVETSSRYCAKRIGVIVRTIPV

SEQ ID NO: 347 - protein, 33 aa, synthetic construct
DEADGVGSRLDAVETSSRYCAKRIGVIARTIPV

SEQ ID NO: 348 - protein, 30 aa, synthetic construct
YHIVRGALDTAGVKDRKQGRSKYGAKRPKS

SEQ ID NO: 349 - protein, 24 aa, synthetic construct
GVSAVLGLLGGGSLTALLLPDAKR

SEQ ID NO: 350 - protein, 35 aa, synthetic construct
KGGNCCENGGTPPPPPAAARSTTPLVANLKPRKKS

SEQ ID NO: 351 - protein, 41 aa, synthetic construct
QALRSYAKPAGSTRIVNFPASQIGVTAPQPTAKATGRKARK

SEQ ID NO: 352 - protein, 13 aa, synthetic construct
NGLKKFFKWVRKL

SEQ ID NO: 353 - protein, 18 aa, synthetic construct
QQLVTLIRGGCAKIIARL

SEQ ID NO: 354 - protein, 17 aa, synthetic construct
LPNLGRRMVGAWCFVVH

SEQ ID NO: 355 - protein, 11 aa, synthetic construct
TGAKIISHFLG

SEQ ID NO: 356 - protein, 25 aa, synthetic construct
DLIDFMRQMISCVVSRRQRYKNSLK

SEQ ID NO: 357 - protein, 33 aa, synthetic construct
YHIIRGALDTAGVADRAQARSKYGAKRPKAAKK

SEQ ID NO: 358 - protein, 18 aa, synthetic construct
SGGGKFIGNLIKLARPLL

SEQ ID NO: 359 - protein, 19 aa, synthetic construct
QMGATAIKQVKKLFKKWGW

SEQ ID NO: 360 - protein, 19 aa, synthetic construct
QMGATAIKQVKKLFKKWGW

SEQ ID NO: 361 - protein, 19 aa, synthetic construct
QMGATAIKQVKKLFKKWGW

SEQ ID NO: 362 - protein, 19 aa, synthetic construct
QMGATAIKQVKKLFKKWGW

SEQ ID NO: 363 - protein, 19 aa, synthetic construct
QMGATAIKQVKKLFKKWGW

SEQ ID NO: 364 - protein, 34 aa, synthetic construct
IVSTIGGAVAGAAGLNPFTTVAGAAIGLSLPRLQ

SEQ ID NO: 365 - protein, 34 aa, synthetic construct
TFFNALARSLSTISDIGRSLGTAIRMLFSKRVCK

SEQ ID NO: 366 - protein, 30 aa, synthetic construct
YHIVRGALDTAGVNDRKQSRSKYGTKRPKA

SEQ ID NO: 367 - protein, 30 aa, synthetic construct
YHIVRGALDTAGVNDRKQGRSKYGTKRPKA

SEQ ID NO: 368 - protein, 30 aa, synthetic construct
YHIVRGALDTAGVADRKQSRSKYGAKRPKG

SEQ ID NO: 369 - protein, 30 aa, synthetic construct
GGVRYAAKAARPSNCRSRCARGISGIGCPS

SEQ ID NO: 370 - protein, 12 aa, synthetic construct
QLYKNLFKVFRK

SEQ ID NO: 371 - protein, 28 aa, synthetic construct
AGRNFGRTLRNVSQMLNSSMRPISIWNV

SEQ ID NO: 372 - protein, 25 aa, synthetic construct
GAWKNFWSSLRKGFYDGKAGRAIRR

SEQ ID NO: 373 - protein, 13 aa, synthetic construct
KTLTKFFKSLIKR

SEQ ID NO: 374 - protein, 24 aa, synthetic construct
WTNMTNMIKKIKKNKKRAHFFGIF

SEQ ID NO: 375 - protein, 17 aa, synthetic construct
VGRGLWENLSNIFKHHK

SEQ ID NO: 376 - protein, 31 aa, synthetic construct; identical to SEQ ID NO: 341

SEQ ID NO: 377 - protein, 19 aa, synthetic construct
QMGATAIKQVKKLFKKWGW

SEQ ID NO: 378 - protein, 14 aa, synthetic construct
SNFIHKIKQIFTHR

SEQ ID NO: 379 - protein, 35 aa, synthetic construct
NIIRTGLKNGDNWATIGKNIGLYFKNKYAGGNFAK

SEQ ID NO: 380 - protein, 25 aa, synthetic construct
SAWKNFWSSLRKGFYDGEAGRAIRR

SEQ ID NO: 381 - protein, 31 aa, synthetic construct
QGVGYAAGFLTSVSMRRAIRKGSASRYSRYY

SEQ ID NO: 382 - protein, 16 aa, synthetic construct
SAVRGFIHGYSSSHQV

SEQ ID NO: 383 - protein, 14 aa, synthetic construct
GSFQSGYQKGWKNI

SEQ ID NO: 384 - protein, 27 aa, synthetic construct
GILGGGGEGIRSDPTRVAPRRFPRSPM

SEQ ID NO: 385 - protein, 15 aa, synthetic construct
KWGRELGQWIGSKIK

SEQ ID NO: 386 - protein, 25 aa, synthetic construct
SWASKATSWLTGPQQPGSPLLKKHR

SEQ ID NO: 387 - protein, 33 aa, synthetic construct
TVGGSYSSACKRVWNAKPGDITIALYCPLPKVK

SEQ ID NO: 388 - protein, 29 aa, synthetic construct
AVGKNVWEFGKGFVKGWNKAKSTKDLRFH

SEQ ID NO: 389 - protein, 15 aa, synthetic construct
KWGKELGRWIGSKIK

SEQ ID NO: 390 - protein, 22 aa, synthetic construct
LESIQGGYGAIFIPHNQVMKQC

SEQ ID NO: 391 - protein, 30 aa, synthetic construct
KKLQKIAGGATKKGGFKRWQCIFTFFGVCK

SEQ ID NO: 392 - protein, 30 aa, synthetic construct; identical to SEQ ID NO: 391

SEQ ID NO: 393 - protein, 30 aa, synthetic construct; identical to SEQ ID NO: 391

SEQ ID NO: 394 - protein, 30 aa, synthetic construct; identical to SEQ ID NO: 391

SEQ ID NO: 395 - protein, 34 aa, synthetic construct
QMINSFVKLVSTLLDLGRTMGSALRYATSKKKCN

SEQ ID NO: 396 - protein, 19 aa, synthetic construct
ELGRSFGSALRRLLKKKYC

SEQ ID NO: 397 - protein, 21 aa, synthetic construct
GGGWLEGLQKVFNVSKPKLLK

SEQ ID NO: 398 - protein, 31 aa, synthetic construct
GTGISSAYAAYTFSPKFHNPGWRRGPISGYK

SEQ ID NO: 399 - protein, 14 aa, synthetic construct
HAFTKLLKALGGHH

SEQ ID NO: 400 - protein, 14 aa, synthetic construct
HAFTKLLKALGGHH

SEQ ID NO: 401 - protein, 14 aa, synthetic construct
HAFTKLLKALGGHH

SEQ ID NO: 402 - protein, 21 aa, synthetic construct
GGGWLEGLQKVFNVSKPKLLK

SEQ ID NO: 403 - protein, 21 aa, synthetic construct
GGGWLEGLQKVFNVSKPKLLK

SEQ ID NO: 404 - protein, 15 aa, synthetic construct
EWGKQLGKWIGSKIK

SEQ ID NO: 405 - protein, 25 aa, synthetic construct
GAWKNFWSSLRKGFYDGEAGKPIRR

SEQ ID NO: 406 - protein, 25 aa, synthetic construct
GAWKNFWSSLRKGFYDGEAGRAIRR

SEQ ID NO: 407 - protein, 25 aa, synthetic construct
GAWKNFWSSLRKGFYDGEAGRAIRR

SEQ ID NO: 408 - protein, 16 aa, synthetic construct
RRITSFFANLFQKRKK

SEQ ID NO: 409 - protein, 25 aa, synthetic construct
LGKLVRAGVDIGIAIGSKGRYKPRH

SEQ ID NO: 410 - protein, 25 aa, synthetic construct
GAWKNFWSSLRKGFYDGEAGRAIRR

SEQ ID NO: 411 - protein, 42 aa, synthetic construct
GIFGQADSVYRTKRGIEKTLFILTIVLVVLFLLVSLLMLKLS

SEQ ID NO: 412 - protein, 25 aa, synthetic construct
GAWKNFWSSLRKGFYDGEAGRAIRR

SEQ ID NO: 413 - protein, 25 aa, synthetic construct
LGKLVRAGVDIGIAIGSKGRYKPRH

SEQ ID NO: 414 - protein, 18 aa, synthetic construct
MEKVSGGASRYQHRRPVR

SEQ ID NO: 415 - protein, 14 aa, synthetic construct
DAFGKGFKEGSHLA

SEQ ID NO: 416 - protein, 25 aa, synthetic construct
GAWKNFWSSLRKGFYDGEAGRAIRR

SEQ ID NO: 417 - protein, 39 aa, synthetic construct
VKSLLGLLGGIMGMRTNQSFIVQFLSLLLKQPGILTWIE

SEQ ID NO: 418 - protein, 18 aa, synthetic construct
MEKVSGGASRYQHRRPVR

SEQ ID NO: 419 - protein, 32 aa, synthetic construct
SGGKGFLSWASKATSWLVGPQQPGSPLLKKHR

SEQ ID NO: 420 - protein, 39 aa, synthetic construct
GGAASAGANGHAAAASGQAKDTCQWSSCNNCHSLKCKVS

SEQ ID NO: 421 - protein, 25 aa, synthetic construct
VAAVGTAGIAAGLTLIGVELRSPSR

SEQ ID NO: 422 - protein, 32 aa, synthetic construct
EILKGGKTLVTPLFHRMRAGKCLVSGVSDFSK

SEQ ID NO: 423 - protein, 34 aa, synthetic construct
NVLNEVFGGVQTELRAGTRADLRVGNATRPARYG

SEQ ID NO: 424 - protein, 12 aa, synthetic construct
NLATGGAAGWKS

SEQ ID NO: 425 - protein, 15 aa, synthetic construct
KWAKSVGKWIASKIK

SEQ ID NO: 426 - protein, 15 aa, synthetic construct
KWAKSVGKWIASKIK

SEQ ID NO: 427 - protein, 15 aa, synthetic construct
KWAKSAGKWIASKIK

SEQ ID NO: 428 - protein, 39 aa, synthetic construct
ISGGGRIAESIGYGIGIGLACIGDSKCAARKLNKLSNKK

SEQ ID NO: 429 - protein, 32 aa, synthetic construct
KYGKKLGGVWGAVIGGVGGAAVCGLAGYVRKG

SEQ ID NO: 430 - protein, 41 aa, synthetic construct
GGESVFSKIGNAVGPAAYWILKGLGNMSDVNQADRINRKKH

SEQ ID NO: 431 - protein, 14 aa, synthetic construct
REYLNKITKWIKHH

SEQ ID NO: 432 - protein, 23 aa, synthetic construct
DFLKGYLHGISAANKHKKGRLGY

SEQ ID NO: 433 - protein, 25 aa, synthetic construct
FENGREFFQAWLKFQQTNPYWQPFY

SEQ ID NO: 434 - protein, 14 aa, synthetic construct
REYLNKITKWIKHH

SEQ ID NO: 435 - protein, 21 aa, synthetic construct
EIGQAFGSAIRRKISNAYCKI

SEQ ID NO: 436 - protein, 42 aa, synthetic construct
AWKNFWSSLRKGFYDGEAGRANPSLINGLKLRRAYSGNQINY

SEQ ID NO: 437 - protein, 22 aa, synthetic construct
DFGNSLGSSIRRIKGKKLCPIR

SEQ ID NO: 438 - protein, 30 aa, synthetic construct
IVQGAVAVFKSGYRHRNDIKAGFSAGFYGK

SEQ ID NO: 439 - protein, 29 aa, synthetic construct
FGGLLRHLFGFKSYSYHQPVKHEPPKSKH

SEQ ID NO: 440 - protein, 24 aa, synthetic construct
SHLPSIFEKFCNWIGMCDNKDMRI

SEQ ID NO: 441 - protein, 23 aa, synthetic construct
IGGGWLEGLQKVFNVSKPKLLKY

SEQ ID NO: 442 - protein, 13 aa, synthetic construct
VVSFGKGIVSAFG

SEQ ID NO: 443 - protein, 25 aa, synthetic construct
GGIGEYYTSYAAILGGSNPLMPTNK

SEQ ID NO: 444 - protein, 23 aa, synthetic construct
GLATGFYGVYRAIEPFIGDEKKR

SEQ ID NO: 445 - protein, 16 aa, synthetic construct
FSSGYKFGTDLARRGR

SEQ ID NO: 446 - protein, 38 aa, synthetic construct
KELAGIVGGGWGGIGIGVIKNWRGEVSAFKRGYRGENY

SEQ ID NO: 447 - protein, 12 aa, synthetic construct
NIGKKIKDIFGR

SEQ ID NO: 448 - protein, 24 aa, synthetic construct
AVIAGVISFISGIFDGFTRPYKCR

SEQ ID NO: 449 - protein, 24 aa, synthetic construct
LIGGLVTLITGMIDGYFRPLKCNK

SEQ ID NO: 450 - protein, 28 aa, synthetic construct
KELKQIVGGCGEITLMAVGGQKKRRIGL

SEQ ID NO: 451 - protein, 12 aa, synthetic construct
TAATGGFGKIRK

SEQ ID NO: 452 - protein, 35 aa, synthetic construct
SFFNSVARILRTAYDIGYSIGSSLRRLFSRNLCKI

SEQ ID NO: 453 - protein, 32 aa, synthetic construct; identical to SEQ ID NO: 429

SEQ ID NO: 454 - protein, 22 aa, synthetic construct
ADRVQDMWNSGNLQCQADNIVA

SEQ ID NO: 455 - protein, 13 aa, synthetic construct
SGFKKGWNGGRRK

SEQ ID NO: 456 - protein, 14 aa, synthetic construct
SNFIHKIKQIFTHR

SEQ ID NO: 457 - protein, 13 aa, synthetic construct
SGFKKGWNGGRRK

SEQ ID NO: 458 - protein, 13 aa, synthetic construct
SGFKKGWNGGRRK

SEQ ID NO: 459 - protein, 28 aa, synthetic construct
SYYRRGWDSVDTRDIVWQCQRYKEKHNV

SEQ ID NO: 460 - protein, 20 aa, synthetic construct
GIYQAGYAAGKDRAQAGLRR

SEQ ID NO: 461 - protein, 19 aa, synthetic construct
KALKKIIDAIVKRPPGQEL

SEQ ID NO: 462 - protein, 37 aa, synthetic construct
LESLGSRARRVLKEISPEAKLELNKSLFDARSRFIER

SEQ ID NO: 463 - protein, 34 aa, synthetic construct
GGFIGRAIGDFVYFGAKGLRESGKLLNYYYKHKH

SEQ ID NO: 464 - protein, 37 aa, synthetic construct
TGATRGVSWCRGFGPWGMTACALGGAAIGGYLGYKSN

SEQ ID NO: 465 - protein, 12 aa, synthetic construct
TAATGGFGKIRK

SEQ ID NO: 466 - protein, 37 aa, synthetic construct
TGATRGVSWCRGFGPWGMTACGLGGAAIGGYLGYKSN

SEQ ID NO: 467 - protein, 12 aa, synthetic construct
TAATGGFGKIRK

SEQ ID NO: 468 - protein, 14 aa, synthetic construct
QAAKAIGKAVGKLF

SEQ ID NO: 469 - protein, 14 aa, synthetic construct
QAAKAIGKAVGKLF

SEQ ID NO: 470 - protein, 35 aa, synthetic construct
KGGNKAITPVREGDCTTTSVTNNCTTTSTTGATNL

SEQ ID NO: 471 - protein, 37 aa, synthetic construct
ASFLNAISRGIETFLNLGRMIGSTIRRIGAKNLCPIS

SEQ ID NO: 472 - protein, 22 aa, synthetic construct
DFVTGLAKGIGKEGNKNKWKNV

SEQ ID NO: 473 - protein, 22 aa, synthetic construct
DFVAGLAKGIGKEGNKNKWKNV

SEQ ID NO: 474 - protein, 13 aa, synthetic construct
GLVGGAISCLNNK

SEQ ID NO: 475 - protein, 25 aa, synthetic construct
SLLNELGQQLGSAIRRKRDGRKCAY

SEQ ID NO: 476 - protein, 29 aa, synthetic construct
SIWAIIGALAGLIFGIGVFEGYTRPIKCR

SEQ ID NO: 477 - protein, 35 aa, synthetic construct
FSKLQKLIGGVEKRTGESCFKICDTNIQNCTWGNA

SEQ ID NO: 478 - protein, 19 aa, synthetic construct
SAGQKLGNMVGKIVPLPFG

SEQ ID NO: 479 - protein, 12 aa, synthetic construct
NLATGGAAGWKS

SEQ ID NO: 480 - protein, 12 aa, synthetic construct
TAATGGFGKIRK

SEQ ID NO: 481 - protein, 21 aa, synthetic construct
AGINSFVSGVASGAGSIGRRP

SEQ ID NO: 482 - protein, 27 aa, synthetic construct
LAGAAGGAVAGGCIKFGYNKTPKHGWN

SEQ ID NO: 483 - protein, 19 aa, synthetic construct
NFGKSYYGKCSFKFLGTCK

SEQ ID NO: 484 - protein, 13 aa, synthetic construct
GLVGGAISCLNNK

SEQ ID NO: 485 - protein, 13 aa, synthetic construct
GLVGGAISCLNNK

SEQ ID NO: 486 - protein, 12 aa, synthetic construct
RGIIGIGKKLFG

SEQ ID NO: 487 - protein, 12 aa, synthetic construct
NLATGGAAGWKS

SEQ ID NO: 488 - protein, 27 aa, synthetic construct
TGGARVNGFVGGKISKNIFYWTRNIWK

SEQ ID NO: 489 - protein, 27 aa, synthetic construct
TGGARVNGFVGGKISKNIFYWTRNIWK

SEQ ID NO: 490 - protein, 27 aa, synthetic construct
TGGARVNGFVGGKISKNIFYWTRNIWK

SEQ ID NO: 491 - protein, 33 aa, synthetic construct
AGFTKVAGAITAGAGVVGAGYVGGKWVGKKIFK

SEQ ID NO: 492 - protein, 33 aa, synthetic construct; identical to SEQ ID NO: 491

SEQ ID NO: 493 - protein, 28 aa, synthetic construct
GWLSMASSALTGPQQPNSPWLAKIKNHK

SEQ ID NO: 494 - protein, 28 aa, synthetic construct
GWLSMASSALTGPQQPNSPWLAKIKNHK

SEQ ID NO: 495 - protein, 12 aa, synthetic construct
NLATGGAAGWKS

SEQ ID NO: 496 - protein, 12 aa, synthetic construct
NLATGGAAGWKS

SEQ ID NO: 497 - protein, 12 aa, synthetic construct
NLATGGAAGWKS

SEQ ID NO: 498 - protein, 12 aa, synthetic construct
NLATGGAAGWKS

SEQ ID NO: 499 - protein, 12 aa, synthetic construct
NLATGGAAGWNK

SEQ ID NO: 500 - protein, 13 aa, synthetic construct
KGFKKGMNHYNKK

SEQ ID NO: 501 - protein, 19 aa, synthetic construct
DIAKGIVSAMNSDNKKKHR

SEQ ID NO: 502 - protein, 33 aa, synthetic construct; identical to SEQ ID NO: 491

SEQ ID NO: 503 - protein, 33 aa, synthetic construct; identical to SEQ ID NO: 491

SEQ ID NO: 504 - protein, 13 aa, synthetic construct
KGFKKGMNHYNKK

SEQ ID NO: 505 - protein, 31 aa, synthetic construct
AAGSGYINTVSGECNSSGRSCWKALKDLLGF

SEQ ID NO: 506 - protein, 24 aa, synthetic construct
YGGNLWKDFDNLMLNKHRYSRFLN

SEQ ID NO: 507 - protein, 12 aa, synthetic construct
RGIIGIGKKLFG

SEQ ID NO: 508 - protein, 32 aa, synthetic construct
LQEIKGGFGAGVGIALFMAAYTIGKDLRKKFG

SEQ ID NO: 509 - protein, 36 aa, synthetic construct
IGGSAKSYIRRLGPDGGYGGRESKLIAMADMIRRRI

SEQ ID NO: 510 - protein, 36 aa, synthetic construct
GGAGAVVGGLGGALLGGSLGVIAGSAVCVGYVASHR

SEQ ID NO: 511 - protein, 18 aa, synthetic construct
SFGTEFWGSANSISKANG

SEQ ID NO: 512 - protein, 24 aa, synthetic construct
KQWYRLGERVGRVAVFIGGGLLFK

SEQ ID NO: 513 - protein, 18 aa, synthetic construct
IRGMIRAVTHLVTVEEVD

SEQ ID NO: 514 - protein, 22 aa, synthetic construct
GLCTGFYGVYKTVEPFLGEGRK

SEQ ID NO: 515 - protein, 12 aa, synthetic construct
RGMIGIGKKLFG

SEQ ID NO: 516 - protein, 12 aa, synthetic construct
RGMIGIGKKLFG

SEQ ID NO: 517 - protein, 19 aa, synthetic construct
IGNNAAANLTTGGKAGWKG

SEQ ID NO: 518 - protein, 13 aa, synthetic construct
SGFKKGWNGGHRK

SEQ ID NO: 519 - protein, 29 aa, synthetic construct
YLGRFLASATHYYGKTVTKGHMHSSTINN

SEQ ID NO: 520 - protein, 29 aa, synthetic construct
YLGRFLASATHYYGKTVTKGHMHSSTINN

SEQ ID NO: 521 - protein, 12 aa, synthetic construct
RGMIGIGKKLFG

SEQ ID NO: 522 - protein, 34 aa, synthetic construct
RIVTALSAFFTSGFTVLLYIIAAIVLPTDKDIKR

SEQ ID NO: 523 - protein, 25 aa, synthetic construct
ANFEQMLGAIDKMNKSGKLDYLKKK

SEQ ID NO: 524 - protein, 39 aa, synthetic construct
STCGGGFINILTIRGAHDGVKGNTEIFLFGIRIYHGKNA

SEQ ID NO: 525 - protein, 16 aa, synthetic construct
FSSGYKFGTDLARRGR

SEQ ID NO: 526 - protein, 35 aa, synthetic construct
KALKKAVSGLGKIIKIIIDLPKPPSPGPTNPGPTF

SEQ ID NO: 527 - protein, 14 aa, synthetic construct
GKSCWKILKDLIGL

SEQ ID NO: 528 - protein, 16 aa, synthetic construct
FSSGYKFGTDLARRGR

SEQ ID NO: 529 - protein, 40 aa, synthetic construct
IVSTIGGAVAGAAGLNPFTTVAGAAIGLSLCLSTNYIHPA

SEQ ID NO: 530 - protein, 40 aa, synthetic construct; identical to SEQ ID NO: 529

SEQ ID NO: 531 - protein, 40 aa, synthetic construct; identical to SEQ ID NO: 529

SEQ ID NO: 532 - protein, 40 aa, synthetic construct; identical to SEQ ID NO: 529

SEQ ID NO: 533 - protein, 17 aa, synthetic construct
RWGAAVGTGGKATIGHC

SEQ ID NO: 534 - protein, 17 aa, synthetic construct
RWGAAVGTGGKATIGHC

SEQ ID NO: 535 - protein, 40 aa, synthetic construct; identical to SEQ ID NO: 529

SEQ ID NO: 536 - protein, 40 aa, synthetic construct; identical to SEQ ID NO: 529

SEQ ID NO: 537 - protein, 40 aa, synthetic construct; identical to SEQ ID NO: 529

SEQ ID NO: 538 - protein, 40 aa, synthetic construct; identical to SEQ ID NO: 529

SEQ ID NO: 539 - protein, 40 aa, synthetic construct; identical to SEQ ID NO: 529

SEQ ID NO: 540 - protein, 40 aa, synthetic construct; identical to SEQ ID NO: 529

SEQ ID NO: 541 - protein, 40 aa, synthetic construct; identical to SEQ ID NO: 529

SEQ ID NO: 542 - protein, 40 aa, synthetic construct; identical to SEQ ID NO: 529

SEQ ID NO: 543 - protein, 40 aa, synthetic construct; identical to SEQ ID NO: 529

SEQ ID NO: 544 - protein, 40 aa, synthetic construct; identical to SEQ ID NO: 529

SEQ ID NO: 545 - protein, 20 aa, synthetic construct
LLGQAKDAVKQYKKDYKRWH

SEQ ID NO: 546 - protein, 33 aa, synthetic construct
GGLEDCINPATGGCRKISIGCAQPQCRPVIDPL

SEQ ID NO: 547 - protein, 28 aa, synthetic construct
DCIDPMTGACSKISIGCAQLQCRPGIEP

SEQ ID NO: 548 - protein, 37 aa, synthetic construct
LKAINGGAGSCAVGECKLRGVSHFIIIGPRGNDGYCC

SEQ ID NO: 549 - protein, 35 aa, synthetic construct
LQEIKGGFGAGVGIALFMAGYTIGKDLRKKFGKSC

SEQ ID NO: 550 - protein, 33 aa, synthetic construct; identical to SEQ ID NO: 491

SEQ ID NO: 551 - protein, 36 aa, synthetic construct
IAGGKAALGDVCVFKSSSCTIKPTEPTQPTQPTTTA

SEQ ID NO: 552 - protein, 27 aa, synthetic construct
GVVQAGVDFVSGVWAGIKQSAKDHPNA

SEQ ID NO: 553 - protein, 27 aa, synthetic construct
TGGARVNGFVNGNWNKGIFYWTRNIWK

SEQ ID NO: 554 - protein, 42 aa, synthetic construct
KYYGNGVHCGKKTCYVDWGQATASIGKIIVNGWTQHGPWAHR

SEQ ID NO: 555 - protein, 38 aa, synthetic construct
QGIAGGAAYVKNKLERSVQRRAIRNPKIVEITGVLVGL

SEQ ID NO: 556 - protein, 33 aa, synthetic construct; identical to SEQ ID NO: 491

SEQ ID NO: 557 - protein, 33 aa, synthetic construct; identical to SEQ ID NO: 491

SEQ ID NO: 558 - protein, 30 aa, synthetic construct
GGYNRLAGRIGHYTGKAALWGIAVAGLFLI

SEQ ID NO: 559 - protein, 34 aa, synthetic construct
NALGNGISGFVSGLLSGFVGVYLVLRKVRVPKPA

SEQ ID NO: 560 - protein, 15 aa, synthetic construct
FNAIGNNGSTFNVTY

SEQ ID NO: 561 - protein, 15 aa, synthetic construct
FNAIGNNGSTFNVTY

SEQ ID NO: 562 - protein, 29 aa, synthetic construct
GGSKGWNKFWKSEGKYWRGFKEGLCKYAR

SEQ ID NO: 563 - protein, 29 aa, synthetic construct
GGSKGWNKFWKSEGKYWRGFKEGLCKYAR

SEQ ID NO: 564 - protein, 18 aa, synthetic construct
IRGMINAVRHLVTVEEVD

SEQ ID NO: 565 - protein, 18 aa, synthetic construct
IRGMINAVRHLVTVEEVD

SEQ ID NO: 566 - protein, 14 aa, synthetic construct
ECVSGVIDWVRKRL

SEQ ID NO: 567 - protein, 35 aa, synthetic construct
IGAAFGNPLTGCVANSAFSFSWQAFKNRPRPKKIA

SEQ ID NO: 568 - protein, 22 aa, synthetic construct
ELGKSLGSSFRRITSGATCSLE

SEQ ID NO: 569 - protein, 41 aa, synthetic construct
DSWEGFGSGLHKTVNTVVYAGTTVARAHTRSHQRCFTGNKW

SEQ ID NO: 570 - protein, 21 aa, synthetic construct
KIGKTVGTIVRKGFEIWSIFK

SEQ ID NO: 571 - protein, 19 aa, synthetic construct
KLVTAFGSEFNDVTGKLGW

SEQ ID NO: 572 - protein, 27 aa, synthetic construct
GGGDTVFDKVNHFFCDLINCRGKGRMN

SEQ ID NO: 573 - protein, 31 aa, synthetic construct
MGGTAAGRCSEVGSKYADSIYVKRPGEELEG

SEQ ID NO: 574 - protein, 37 aa, synthetic construct
GGSDLGDLWTGARDWLWGFKNGFEGRARHHRSWRDRD

SEQ ID NO: 575 - protein, 32 aa, synthetic construct
ASYYRKGWDSVDVRDIVWQCHRYKEKKNVQLN

SEQ ID NO: 576 - protein, 23 aa, synthetic construct
SVLAALCNTINPLDCSMSAFCAY

SEQ ID NO: 577 - protein, 22 aa, synthetic construct
GGGWLEGLQKVFNVSKPKLLEW

SEQ ID NO: 578 - protein, 20 aa, synthetic construct
NAWKSLWKGFWESLRYTDGF

SEQ ID NO: 579 - protein, 22 aa, synthetic construct
GGGWLEGLQKVFNVSKPKLLEW

SEQ ID NO: 580 - protein, 21 aa, synthetic construct
SVVAAIIGGAAGFVVGKYFKK

SEQ ID NO: 581 - protein, 30 aa, synthetic construct
AGGAAIIDYGNDFVKGAKKGAKKAGNKSWL

SEQ ID NO: 582 - protein, 17 aa, synthetic construct
NVFKSVVDIARRGVIVY

SEQ ID NO: 583 - protein, 36 aa, synthetic construct
KGGAKCGAAIAGGLFGIPKGPLAWAAGLANVYSKCN

SEQ ID NO: 584 - protein, 36 aa, synthetic construct; identical to SEQ ID NO: 583

SEQ ID NO: 585 - protein, 36 aa, synthetic construct; identical to SEQ ID NO: 583

SEQ ID NO: 586 - protein, 36 aa, synthetic construct; identical to SEQ ID NO: 583

SEQ ID NO: 587 - protein, 42 aa, synthetic construct
TVAGAAGGGLAAGLGAIPGAFVGAHVGVIGGSAACIGGFLGN

SEQ ID NO: 588 - protein, 29 aa, synthetic construct
ASYYRRGWDSVDIRDIVWQCQRYKEKHND

SEQ ID NO: 589 - protein, 19 aa, synthetic construct
DGIIGGIDKVIDGVIPRKP

SEQ ID NO: 590 - protein, 19 aa, synthetic construct
DGIIGGIDKVIDGIIPRKP

SEQ ID NO: 591 - protein, 29 aa, synthetic construct
ASYYRRGWDSVDIRDIVWQCQRYKEKHND

SEQ ID NO: 592 - protein, 41 aa, synthetic construct
NAATAAASGAVKGILGGPTGALAGAIWGVSQCASNNLHGMH

SEQ ID NO: 593 - protein, 20 aa, synthetic construct
IGNNAAANLTTGGAAGWNKG

SEQ ID NO: 594 - protein, 19 aa, synthetic construct
IGNNAVANLTTGGAAGWKS

SEQ ID NO: 595 - protein, 19 aa, synthetic construct
IGNNAVANLTTGGAAGWKS

SEQ ID NO: 596 - protein, 14 aa, synthetic construct
GNGGGGSYADIICV

SEQ ID NO: 597 - protein, 27 aa, synthetic construct
GKLRAWGGGCDSKCPDGMTPFCDVSRN

SEQ ID NO: 598 - protein, 41 aa, synthetic construct
ALQGLFGGFANSPTLEQLNGTKLPKPKSCSPYGTGGTSNAC

SEQ ID NO: 599 - protein, 32 aa, synthetic construct
WIGKVGKQYKKATDNYNKNRNKYCGNLSRKDC

SEQ ID NO: 600 - protein, 17 aa, synthetic construct
SIIGGMISGWASGLAGM

SEQ ID NO: 601 - protein, 17 aa, synthetic construct
SIIGGMISGWASGLAGM

SEQ ID NO: 602 - protein, 14 aa, synthetic construct
VIGGWASSLAGMGH

SEQ ID NO: 603 - protein, 25 aa, synthetic construct
IAGRVGAAYGHAQASANNHHSPING

SEQ ID NO: 604 - protein, 42 aa, synthetic construct; identical to SEQ ID NO: 554

SEQ ID NO: 605 - protein, 39 aa, synthetic construct
GTFGGSLPNFLTAFIGGRKLSKNQVEKLKKLIDEHKEVQ

SEQ ID NO: 606 - protein, 32 aa, synthetic construct
SIVTEGGGMGGTYGYVTSCALNDYTECLSRLS

SEQ ID NO: 607 - protein, 24 aa, synthetic construct
VAKDAWNHLDQIRSGWRKAGNSKW

SEQ ID NO: 608 - protein, 25 aa, synthetic construct
AEGIGNDLNDLYRGARDAWKATRRR

SEQ ID NO: 609 - protein, 33 aa, synthetic construct
CNSLCGPGGGVISNRPGVGDACNPDRSICCICY

SEQ ID NO: 610 - protein, 35 aa, synthetic construct
LAAYGGALATFLLADAWAHTDQIFKGFKKGWNGGR

SEQ ID NO: 611 - protein, 14 aa, synthetic construct
GNGGGGGYGDIICA

SEQ ID NO: 612 - protein, 42 aa, synthetic construct
HELIKIYGGAINGTLINAFARLLDLAIEIGKMIGYSLQRLIK

SEQ ID NO: 613 - protein, 33 aa, synthetic construct
NRLVQVVGGWLHSFALVTYFCMLLGFHSFTAYR

SEQ ID NO: 614 - protein, 31 aa, synthetic construct
TLCGPAGGVISNKPGRGDACNPDRSICCFCH

SEQ ID NO: 615 - protein, 13 aa, synthetic construct
LSRVKGGMSRIAG

SEQ ID NO: 616 - protein, 36 aa, synthetic construct
YSSISACASACGVTVNPSTTTPTTGPSTPVLTKPMQ

SEQ ID NO: 617 - protein, 27 aa, synthetic construct
GECRKYGRGCAEQQCQLIIIDPIERLF

SEQ ID NO: 618 - protein, 30 aa, synthetic construct
RCRKVYHRLWGKSYGGWNNGMSCGYDGTKA

SEQ ID NO: 619 - protein, 39 aa, synthetic construct
LKTIKGGGDQGVCYLPGGGYVIRDCSSRCPNGGYPACPA

SEQ ID NO: 620 - protein, 42 aa, synthetic construct
ATGAALGSAICGPGCGFVGAHWGAVGWTAVASFSGAFGKIRK

SEQ ID NO: 621 - protein, 13 aa, synthetic construct
ALLGAGFGMWTGA

SEQ ID NO: 622 - protein, 12 aa, synthetic construct
VKAFIEKCRKVY

SEQ ID NO: 623 - protein, 24 aa, synthetic construct
GAVDTVFGIIGNGWGSAGASGRQH

SEQ ID NO: 624 - protein, 24 aa, synthetic construct
GAVDTVFGIIGNGWGSAGASGRQH

SEQ ID NO: 625 - protein, 20 aa, synthetic construct
NAWKSLWKGFWESLRYTDGF

SEQ ID NO: 626 - protein, 20 aa, synthetic construct
NAWKSLWKGFWESLRYTDGF

SEQ ID NO: 627 - protein, 22 aa, synthetic construct
RFFGGIYNAGRSFGRNVYNAFY

SEQ ID NO: 628 - protein, 12 aa, synthetic construct
RLPHIIKKLFSK

SEQ ID NO: 629 - protein, 22 aa, synthetic construct
RFFGGIYNAGRSFGRNVYNAFY

SEQ ID NO: 630 - protein, 29 aa, synthetic construct
GEKIGKLLREVREAKNPARDLTEIFSLKG

SEQ ID NO: 631 - protein, 29 aa, synthetic construct
GSVFHTGTTVGTDTGVSQNVIEDVKDSYN

SEQ ID NO: 632 - protein, 42 aa, synthetic construct
AIGSTVAGAGLGGAICGPACAVVGAHYGPIIWAGVSGATGAF

SEQ ID NO: 633 - protein, 20 aa, synthetic construct
IGNNAAANLTTGGAAGWNKG

SEQ ID NO: 634 - protein, 20 aa, synthetic construct
AGAVGGAIIGGVGYGATCWW

SEQ ID NO: 635 - protein, 21 aa, synthetic construct
DMGRALGSSIRRLVNNNMCDF

SEQ ID NO: 636 - protein, 20 aa, synthetic construct
ATVNTVVNGWMRYGPWIPRP

SEQ ID NO: 637 - protein, 20 aa, synthetic construct
ATVNTVVNGWMRYGPWIPRP

SEQ ID NO: 638 - protein, 15 aa, synthetic construct
YKNVKAFIEKCRSSY

SEQ ID NO: 639 - protein, 33 aa, synthetic construct
IAERAADLIRRGGRPVLNVVTDAPAQSVPAGVR

SEQ ID NO: 640 - protein, 33 aa, synthetic construct; identical to SEQ ID NO: 639

SEQ ID NO: 641 - protein, 42 aa, synthetic construct
TVAGAAGGGLAAGLGAIPGAFVGAHFGVIGGSAACIGGLLGN

SEQ ID NO: 642 - protein, 40 aa, synthetic construct
SAASGAGTGIKACKSFGPWGMAICGVGGAAIGGYFGYTHN

SEQ ID NO: 643 - protein, 42 aa, synthetic construct; identical to SEQ ID NO: 641

SEQ ID NO: 644 - protein, 42 aa, synthetic construct
GVGAVGGAIKGGIQTGTWQGAALKGIGYGIKGGITYGLTCRY

SEQ ID NO: 645 - protein, 27 aa, synthetic construct
GGGDTVFDKVNHFFCDLINCRGKGRMN

SEQ ID NO: 646 - protein, 40 aa, synthetic construct; identical to SEQ ID NO: 642

SEQ ID NO: 647 - protein, 42 aa, synthetic construct
GVGAVGGAVKGGIQTGTWQGAALKGIGYGIKGGITYGLTCRY

SEQ ID NO: 648 - protein, 14 aa, synthetic construct
SCGNKVKNGLRDYN

SEQ ID NO: 649 - protein, 20 aa, synthetic construct
ATVNTVVNGWMKYGPWIPRR

SEQ ID NO: 650 - protein, 31 aa, synthetic construct
GLLGGIFGLLRKPAAYCKPAPVCAPKPKHGC

SEQ ID NO: 651 - protein, 18 aa, synthetic construct
EAASAGIHRLANGGNGFW

SEQ ID NO: 652 - protein, 15 aa, synthetic construct
AGNLIRGFKDVWKNN

SEQ ID NO: 653 - protein, 42 aa, synthetic construct
  SITE 13: note = misc_feature - Xaa can be any naturally occurring amino acid
GNAATAAASGAVXGILGGPTGALAGAIWGVSQCASNNLHGMH

SEQ ID NO: 654 - protein, 40 aa, synthetic construct
GGLGGLQTGIKYCKVPHPVLKGVCIVGFTTAGAYLAYKAN

SEQ ID NO: 655 - protein, 24 aa, synthetic construct
GAGRSGCAPGTQLAGDGIQRLAGG

SEQ ID NO: 656 - protein, 27 aa, synthetic construct
SAFGAAWGLAKASVLYCGLGPQPQKVY

SEQ ID NO: 657 - protein, 23 aa, synthetic construct
DAGRSVGSGIRRATSDSLCPTDL

SEQ ID NO: 658 - protein, 32 aa, synthetic construct
GWWQAGLAIAGAVIYLYNEGGDFIKGYKEGRK

SEQ ID NO: 659 - protein, 14 aa, synthetic construct
GSACAYMRRVCKKN

SEQ ID NO: 660 - protein, 31 aa, synthetic construct
TLCGPGGGVISNTPGVGDACSPDRKVCCICY

SEQ ID NO: 661 - protein, 14 aa, synthetic construct
IWTPAKNLYNKIFR

SEQ ID NO: 662 - protein, 38 aa, synthetic construct
VAVGDAAFDNWFVLAHFLSPRLLLSPRHKPDIPRQNAR

SEQ ID NO: 663 - protein, 38 aa, synthetic construct
MAQLRGGVRQLDESCAAFSCNTSCKSGSCNSKTTEEIS

SEQ ID NO: 664 - protein, 32 aa, synthetic construct
GGKQGIDNYIENSIASARSSINYWNRRILGKS

SEQ ID NO: 665 - protein, 33 aa, synthetic construct
SGGAGYVKPGGVFSLTNPSATLPRKSNQYYQAS

SEQ ID NO: 666 - protein, 33 aa, synthetic construct
GGMQEIAKTCAAGSCLNSCSSGSCNTKAVEEIS

SEQ ID NO: 667 - protein, 33 aa, synthetic construct
GGMQEIAKTCAAGSCLNSCNSGSCNTKAVEEIS

SEQ ID NO: 668 - protein, 17 aa, synthetic construct
NAAANWATGGAAGWNKG

SEQ ID NO: 669 - protein, 37 aa, synthetic construct
KYYGNGVTCGKHSCSVNWGQAFSCSVSHLANFGHGKC

SEQ ID NO: 670 - protein, 17 aa, synthetic construct
NLVSGLYDGWKSKHKKK

SEQ ID NO: 671 - protein, 29 aa, synthetic construct
QVAREAGAAGREIKDEFMKGREEGKKEGK

SEQ ID NO: 672 - protein, 38 aa, synthetic construct
MAQLKGGIKSVEGSCGAGSCAHSCNKDSCNTKAIEEIS

SEQ ID NO: 673 - protein, 38 aa, synthetic construct
MAQLKGGIKNADGSCGVASCGSSCKNDSCNTKAVEEVS

SEQ ID NO: 674 - protein, 38 aa, synthetic construct
MAQLKGGIKNADGSCGVASCGSSCNKDSCNTKTVEELS

SEQ ID NO: 675 - protein, 18 aa, synthetic construct
TGPKYCSTYIGCPQVITC

SEQ ID NO: 676 - protein, 19 aa, synthetic construct
IIGAISGYINPVKCNGSSK

SEQ ID NO: 677 - protein, 33 aa, synthetic construct
SYCTDLITNYNNNHTKWSKEVQEIALDMMQAHC

SEQ ID NO: 678 - protein, 32 aa, synthetic construct
ITTIIGTIITFIVGTLKGYSNPEKCNVLGGKR

SEQ ID NO: 679 - protein, 27 aa, synthetic construct
LSTLVGGGSTCAGLTCSWCTSTMSSRS

SEQ ID NO: 680 - protein, 36 aa, synthetic construct
CLQLITQFVEALPEDYCAYPENAAHIACQCKRLYGL

SEQ ID NO: 681 - protein, 31 aa, synthetic construct
GGNGGNGGRGGNGGNGGNGGNCGGGNNGGRR

SEQ ID NO: 682 - protein, 18 aa, synthetic construct
GFGKGAIKEGNKDKWKNI

SEQ ID NO: 683 - protein, 27 aa, synthetic construct
LAQMWDSVGRIMYTGWQKDGPFAHPLV

SEQ ID NO: 684 - protein, 27 aa, synthetic construct
LAQMWDSVGRIMYTGWQKDGPFAHPLV

SEQ ID NO: 685 - protein, 27 aa, synthetic construct
LAQMWDSVGRIMYTGWQKDGPFAHPLV

SEQ ID NO: 686 - protein, 29 aa, synthetic construct
KAVTALYDLGKGFGSALRRAISGNYCPVR

SEQ ID NO: 687 - protein, 27 aa, synthetic construct
LAQMWDSVGRIMYTGWQKDGPFAHPLV

SEQ ID NO: 688 - protein, 37 aa, synthetic construct
GTILNQLNKLISILVDSGKSLGSSIRRISEDKICPLD

SEQ ID NO: 689 - protein, 34 aa, synthetic construct
QEMSTLFGGGSTCSSFTCTSCAATLSSKSIQQFD

SEQ ID NO: 690 - protein, 22 aa, synthetic construct
GAGAGLWDAYEESQAKKKKKKK

SEQ ID NO: 691 - protein, 22 aa, synthetic construct
GAGAGLWDAYEESQAKKKKKKK

SEQ ID NO: 692 - protein, 22 aa, synthetic construct
GAGAGMWDAYEESQAKKKKKKK

SEQ ID NO: 693 - protein, 42 aa, synthetic construct
VAGGLRSCMDGCGPGECCSKGVCRSGPNTGPRPYLCDPPILD

SEQ ID NO: 694 - protein, 42 aa, synthetic construct
VSGGLRSCMDGCGPGECCSKGVCRSGPNTGPRPYLCDPPIIE

SEQ ID NO: 695 - protein, 42 aa, synthetic construct; identical to SEQ ID NO: 694

SEQ ID NO: 696 - protein, 37 aa, synthetic construct
AAAAFFSFGTAILLYIIGSLIIPQESYGGFDDSFNNY

SEQ ID NO: 697 - protein, 26 aa, synthetic construct
GNCATEFCTQISFSCGQRDCWPTPIE

SEQ ID NO: 698 - protein, 31 aa, synthetic construct
TLCGEAGGVVSNHPGIGDMCTPDRSLCCYCR

SEQ ID NO: 699 - protein, 31 aa, synthetic construct
CANGVCRPIAGAGPNTYYCTPPTMIDFTPQG

SEQ ID NO: 700 - protein, 34 aa, synthetic construct
GTSAVTVCRRVATCQGKFGEFLTREPADLARCGA

SEQ ID NO: 701 - protein, 34 aa, synthetic construct
GTSAVTVCRRVATCQGKFGDFLTREPADLARCGA

SEQ ID NO: 702 - protein, 13 aa, synthetic construct
GVNHLANGGHGVC

SEQ ID NO: 703 - protein, 31 aa, synthetic construct
KKGLRQITGGMECVCGRPCPDPNQPPPKECD

SEQ ID NO: 704 - protein, 31 aa, synthetic construct
KKGLRQITGGMECMCGRPCPDPNQPPPKECD

SEQ ID NO: 705 - protein, 21 aa, synthetic construct
GGCRSIAGAGPKTYLCYAPMY

SEQ ID NO: 706 - protein, 40 aa, synthetic construct; identical to SEQ ID NO: 642

SEQ ID NO: 707 - protein, 18 aa, synthetic construct
EAFSAGVHRLANGGNGFW

SEQ ID NO: 708 - protein, 18 aa, synthetic construct
EAFSAGVHRLANGGNGFW

SEQ ID NO: 709 - protein, 18 aa, synthetic construct
EAASAGIHRLANGGNGFW

SEQ ID NO: 710 - protein, 32 aa, synthetic construct
SICASGYGLVATGCQGTSYPMLDGYTCVLTCL

SEQ ID NO: 711 - protein, 22 aa, synthetic construct
TGPEYCSTYFGCPQVYTCGTNI

SEQ ID NO: 712 - protein, 29 aa, synthetic construct
AVIGGALGGLGGAGGGCITGAIGSIWDQW

SEQ ID NO: 713 - protein, 29 aa, synthetic construct
AVIGGALGGLGGAGGGCITGAIGSIWDQW

SEQ ID NO: 714 - protein, 15 aa, synthetic construct
GWLAGAAGVINSCMK

SEQ ID NO: 715 - protein, 27 aa, synthetic construct
DWGTAIGNIGNNAAANWATGGNAGWNK

SEQ ID NO: 716 - protein, 41 aa, synthetic construct
SGATAGVKLCSAGGPYAIAACGVGGALLGAGFGMWTGAVNN

SEQ ID NO: 717 - protein, 38 aa, synthetic construct
SGATAGVKLCSAGGPYAIAACGVGGALLGAGFSMWTGA

SEQ ID NO: 718 - protein, 20 aa, synthetic construct
AGAVGGAIIGGVGYGATCWW

SEQ ID NO: 719 - protein, 38 aa, synthetic construct
SGATAGVKLCSAGGPYAIAACGVGGALLGAGFGMWTGA

SEQ ID NO: 720 - protein, 38 aa, synthetic construct; identical to SEQ ID NO: 717

SEQ ID NO: 721 - protein, 20 aa, synthetic construct
AGAVGGAIIGGVGYGATCWG

SEQ ID NO: 722 - protein, 38 aa, synthetic construct; identical to SEQ ID NO: 717

SEQ ID NO: 723 - protein, 38 aa, synthetic construct; identical to SEQ ID NO: 717

SEQ ID NO: 724 - protein, 41 aa, synthetic construct; identical to SEQ ID NO: 716

SEQ ID NO: 725 - protein, 17 aa, synthetic construct
SIIGGMISGWASGLAGM

SEQ ID NO: 726 - protein, 27 aa, synthetic construct
GFSGIGTLISGFVGLAGFGMSLRCRVL

SEQ ID NO: 727 - protein, 38 aa, synthetic construct; identical to SEQ ID NO: 719

SEQ ID NO: 728 - protein, 35 aa, synthetic construct
TFLSSFARMISSIYDVGNNLGSSIRRIVEKRMCSY

SEQ ID NO: 729 - protein, 38 aa, synthetic construct; identical to SEQ ID NO: 719

SEQ ID NO: 730 - protein, 29 aa, synthetic construct
AFLAAVGQQWAATLGITAAAAAGGCFDYW

SEQ ID NO: 731 - protein, 40 aa, synthetic construct
SGIAGGAGNGLRLGIKTRTWQGVVAGAIIGGVGYGATCWW

SEQ ID NO: 732 - protein, 20 aa, synthetic construct
AGAVGGAIIGGVGYGATCWW

SEQ ID NO: 733 - protein, 20 aa, synthetic construct
AGAVGGAIIGGVGYGATCWW

SEQ ID NO: 734 - protein, 11 aa, synthetic construct
TAGAAYSGCFD

SEQ ID NO: 735 - protein, 14 aa, synthetic construct
VIGGWASSLAGMGH

SEQ ID NO: 736 - protein, 26 aa, synthetic construct
MKTCKGCGASILSTENYCEKCKKKQG

SEQ ID NO: 737 - protein, 42 aa, synthetic construct
GVGAVGGAIKGGIQTGTWQGAALKGIGYGIKDGITYGLICRY

SEQ ID NO: 738 - protein, 42 aa, synthetic construct; identical to SEQ ID NO: 737

SEQ ID NO: 739 - protein, 42 aa, synthetic construct; identical to SEQ ID NO: 737

SEQ ID NO: 740 - protein, 42 aa, synthetic construct; identical to SEQ ID NO: 641

SEQ ID NO: 741 - protein, 41 aa, synthetic construct
GGYGDMCNMGNNDSCAQYGLECGLYMGHDHSVGNWYALRCM

SEQ ID NO: 742 - protein, 42 aa, synthetic construct; identical to SEQ ID NO: 737

SEQ ID NO: 743 - protein, 42 aa, synthetic construct; identical to SEQ ID NO: 737

SEQ ID NO: 744 - protein, 42 aa, synthetic construct; identical to SEQ ID NO: 737

SEQ ID NO: 745 - protein, 42 aa, synthetic construct; identical to SEQ ID NO: 737

SEQ ID NO: 746 - protein, 28 aa, synthetic construct
VNEIKGGLTAMMCNTVPEWDGGIGCHLW

SEQ ID NO: 747 - protein, 37 aa, synthetic construct
LQGIEGGCGHLNIMNIIERDGCRPKGHRWKWFWQDEY

SEQ ID NO: 748 - protein, 39 aa, synthetic construct
DAWEKIGEWVGEIGSIWGNSNKKSWEICQKKGIKNRRDC

SEQ ID NO: 749 - protein, 42 aa, synthetic construct; identical to SEQ ID NO: 647

SEQ ID NO: 750 - protein, 37 aa, synthetic construct
NPGETIGDGLSRECGEECGPDNGCGDGYPKCDGVCKK

SEQ ID NO: 751 - protein, 35 aa, synthetic construct
IKGGGDEGYNFWYGIGKWARQQYRGFCNWSNWCKG

SEQ ID NO: 752 - protein, 21 aa, synthetic construct
SFIASYLGSAQTRCGKDNKKK

SEQ ID NO: 753 - protein, 21 aa, synthetic construct
SFIASYLGSAQTRCGKDNKKK

SEQ ID NO: 754 - protein, 21 aa, synthetic construct
SFIASYLGSAQTRCGKDNKKK

SEQ ID NO: 755 - protein, 21 aa, synthetic construct
SFIASYLGSAQTRCGKDNKKK

SEQ ID NO: 756 - protein, 21 aa, synthetic construct
SFIASYLGSAQTRCGKDNKKK

SEQ ID NO: 757 - protein, 21 aa, synthetic construct
SFIASYLGSAQTRCGKDNKKK

SEQ ID NO: 758 - protein, 22 aa, synthetic construct
VDIVGAFVDRLCIERISRSSFS

SEQ ID NO: 759 - protein, 41 aa, synthetic construct
GIAGGIAANCPSMFQSCEEWCSWTPWQKSHCILSEPCTECF

SEQ ID NO: 760 - protein, 35 aa, synthetic construct
TYVQSYMNELRAEGHSDVEAIAILRGKLLACGIHP

SEQ ID NO: 761 - protein, 28 aa, synthetic construct
GNCATEFCTQISFSCGQRDCWPTPIEIE

SEQ ID NO: 762 - protein, 28 aa, synthetic construct
GNCATEFCTQISFSCGQRDCWPTPIEIE

SEQ ID NO: 763 - protein, 28 aa, synthetic construct
GNCATEFCTQISFSCGQRDCWPTPIEIE

SEQ ID NO: 764 - protein, 28 aa, synthetic construct
GNCATEFCTQISFSCGQRDCWPTPIEIE

SEQ ID NO: 765 - protein, 23 aa, synthetic construct
AAFEALPQCCKSKVLVSIDCFPQ

SEQ ID NO: 766 - protein, 41 aa, synthetic construct
QVANTYAGKCSCMCEGSGVYSYYNTINSEAHLYKSNGTIVK

SEQ ID NO: 767 - protein, 41 aa, synthetic construct; identical to SEQ ID NO: 766

SEQ ID NO: 768 - protein, 18 aa, synthetic construct
RACTLISSGCAQKICRPQ

SEQ ID NO: 769 - protein, 21 aa, synthetic construct
WCEKLSACVQSGQYAKNCKSL

SEQ ID NO: 770 - protein, 21 aa, synthetic construct
GGCRSLAGAGPKTYLCYAPMY

SEQ ID NO: 771 - protein, 21 aa, synthetic construct
GGCRSIAGAGPKTYLCYAPMY

SEQ ID NO: 772 - protein, 19 aa, synthetic construct
QWVELVEKVAAAGIGVKGE

SEQ ID NO: 773 - protein, 36 aa, synthetic construct
GAFAGAGGLIGAAAGAGWMAGTFIHNRLVSAYPRIF

SEQ ID NO: 774 - protein, 41 aa, synthetic construct
TAGGKVFNPGGGDGPGEDGCGFNTCRNQWGRCSWAACDRGY

SEQ ID NO: 775 - protein, 27 aa, synthetic construct
CAKSGNAAGAAVMSAHGAAGSMTVVCN

SEQ ID NO: 776 - protein, 27 aa, synthetic construct
ASAAQTAVSKVHMNQDVIRDEVAVVCY

SEQ ID NO: 777 - protein, 27 aa, synthetic construct
ASAAQTAVSKVHMNQDVIRDEVAVVCY

SEQ ID NO: 778 - protein, 20 aa, synthetic construct
VAIIKKYMDEAQIAATPQCF

SEQ ID NO: 779 - protein, 42 aa, synthetic construct
KGGGNVCDEFPKKYQHVSCSEYFNIPPKYSHCVLVDVECFPE

SEQ ID NO: 780 - protein, 14 aa, synthetic construct
GYCSKCAEKIRNGQ

SEQ ID NO: 781 - protein, 24 aa, synthetic construct
DAYYNVISDVAKFTKSNGTVLPNK

SEQ ID NO: 782 - protein, 16 aa, synthetic construct
AFWQGIGRWLDQHFGW

SEQ ID NO: 783 - protein, 24 aa, synthetic construct
DAYYNVISDVAKFTKSNGTVLPNK

SEQ ID NO: 784 - protein, 24 aa, synthetic construct
DAYYNVISDVAKFTKSNGTVLPNK

SEQ ID NO: 785 - protein, 24 aa, synthetic construct
DAYYNVISDVAKFTKSNGTVLPNK

SEQ ID NO: 786 - protein, 24 aa, synthetic construct
DAYYNVISDVAKFTKSNGTVLPNK

SEQ ID NO: 787 - protein, 32 aa, synthetic construct
CTSCINYLNGAACYNSHDKSIHVEKCLELPFV

SEQ ID NO: 788 - protein, 22 aa, synthetic construct
WCEKLSACVQSGQYAKSCKSLE

SEQ ID NO: 789 - protein, 22 aa, synthetic construct
WCEKLSACVQSGQYAKSCKSLE

SEQ ID NO: 790 - protein, 22 aa, synthetic construct
WCEKLSACVQSGQYAKSCKSLE

SEQ ID NO: 791 - protein, 13 aa, synthetic construct
GVCGSAPKCLATS

SEQ ID NO: 792 - protein, 25 aa, synthetic construct
NCVTEYCTEISLACAQTECQPVLEL

SEQ ID NO: 793 - protein, 32 aa, synthetic construct
NIFKSIWNWIKEHIFLETSGDPNARIRGGIRF

SEQ ID NO: 794 - protein, 30 aa, synthetic construct
GAIAAILKDFHIPLSWPSQTAPAAQDPRLL

SEQ ID NO: 795 - protein, 18 aa, synthetic construct
QIAQNLGNYFAKVLLRRS

SEQ ID NO: 796 - protein, 22 aa, synthetic construct
SGGNGAWGWVGQLAPAFSKKRP

SEQ ID NO: 797 - protein, 13 aa, synthetic construct
NQYRNAGRHWARN

SEQ ID NO: 798 - protein, 22 aa, synthetic construct
SGGNGAWGWIGQLAPAFSKKRS

SEQ ID NO: 799 - protein, 22 aa, synthetic construct
SGGNGAWGWIGQLAPAFSKKRS

SEQ ID NO: 800 - protein, 17 aa, synthetic construct
GFAGGIATGVSRKNRKK

SEQ ID NO: 801 - protein, 23 aa, synthetic construct
GNFLREAKNLAGDLIETFSPLKG

SEQ ID NO: 802 - protein, 35 aa, synthetic construct
DEGGASLIGIGGIGDGVPHNAYLRIFTFKIRLPWW

SEQ ID NO: 803 - protein, 12 aa, synthetic construct
DFGRGFVDGFRG

SEQ ID NO: 804 - protein, 12 aa, synthetic construct
DFGRGFVDGFRG

SEQ ID NO: 805 - protein, 18 aa, synthetic construct
KIAKSIIDAFRHAGEIWA

SEQ ID NO: 806 - protein, 27 aa, synthetic construct
GIIDAAIDFGKGFLDGAKNAKHIGGGY

SEQ ID NO: 807 - protein, 17 aa, synthetic construct
AAGDAIVSFGEGFLNAW

SEQ ID NO: 808 - protein, 17 aa, synthetic construct
AAGDAIVSFGEGFLNAW

SEQ ID NO: 809 - protein, 37 aa, synthetic construct
GGFIESAVAKGAMVTLLYAIRPNPKPIIDFIFKVLKK

SEQ ID NO: 810 - protein, 19 aa, synthetic construct
DYWNGVFDGIKSVFNQNKK

SEQ ID NO: 811 - protein, 41 aa, synthetic construct
LGALLGSLGAHLQFNPLLAVAGGALLAGIVISGFTECRLGR

SEQ ID NO: 812 - protein, 28 aa, synthetic construct
LGLAAAIAGGITFLIGIIDGFFRPLSCR

SEQ ID NO: 813 - protein, 18 aa, synthetic construct
GFGKGAIKEGNKDKWKNI

SEQ ID NO: 814 - protein, 26 aa, synthetic construct
SWANYLSRYLKEDYKYFLSDGSKNLL

SEQ ID NO: 815 - protein, 22 aa, synthetic construct
GAGAGLWDAYEESQAKKKKKKK

SEQ ID NO: 816 - protein, 40 aa, synthetic construct
ELMKGLGKGIREFNNAKANIESEVKESMKEIDDKEMKSNK

SEQ ID NO: 817 - protein, 22 aa, synthetic construct
GAGAGLWDAYEESQAKKKKKKK

SEQ ID NO: 818 - protein, 22 aa, synthetic construct
GAGAGLWDAYEESQAKKKKKKK

SEQ ID NO: 819 - protein, 31 aa, synthetic construct
LGKAYKWIANLIESIFYTLNGIPVSDLYYRG

SEQ ID NO: 820 - protein, 42 aa, synthetic construct
SGMTGGGGVVAGSYGGLGGMIVSGMGGLIAGSGNCFGNSGGR

SEQ ID NO: 821 - protein, 22 aa, synthetic construct
VLGAIGAFLAGVLDGYLRPKCN

SEQ ID NO: 822 - protein, 17 aa, synthetic construct
RQMVRHLRRFMRRYGRC

SEQ ID NO: 823 - protein, 31 aa, synthetic construct
AVITTVGGEGALRCRFLDMGLIPKTKVVVKK

SEQ ID NO: 824 - protein, 39 aa, synthetic construct
GGGHAAAAGVRKRTNDVEKFRQEILEWIVMMRKFSGQDK

SEQ ID NO: 825 - protein, 20 aa, synthetic construct
YEKINGVLTRVFTFSCEVVS

SEQ ID NO: 826 - protein, 36 aa, synthetic construct
LDSINGGGKKLWYLLGAGLSFFLGLFSGLVNPVKCN

SEQ ID NO: 827 - protein, 39 aa, synthetic construct
GGGDAAAAGVRKRTNDVEKFRQEILERIVMMRKFFGQDK

SEQ ID NO: 828 - protein, 36 aa, synthetic construct
AEKMNNLWGGGGIIMNTTDVSGRPQCDTRETAMLTL

SEQ ID NO: 829 - protein, 26 aa, synthetic construct
TQVKQFFEKGAKSAMENKTQLMKFLQ

SEQ ID NO: 830 - protein, 27 aa, synthetic construct
ELVKDVVNSLNKWAQSCYAGYKDSPFH

SEQ ID NO: 831 - protein, 26 aa, synthetic construct
AKVKQFFEKGAKSAMENKTQLMKFLQ

SEQ ID NO: 832 - protein, 13 aa, synthetic construct
DAAKDAIKTFTKL

SEQ ID NO: 833 - protein, 13 aa, synthetic construct
GVYNGIKDWFGAC

SEQ ID NO: 834 - protein, 38 aa, synthetic construct
GIAGTGAGAAGGPAGAFVGAHVGVIAGSAVCIGGFLGQ

SEQ ID NO: 835 - protein, 27 aa, synthetic construct
DWGTAIGNIGNNAAANWATGGNAGWNK

SEQ ID NO: 836 - protein, 40 aa, synthetic construct
NMGSAIGGCIGGVLLAAATGPITGGGAAMICVASGISAYL

SEQ ID NO: 837 - protein, 40 aa, synthetic construct; identical to SEQ ID NO: 836

SEQ ID NO: 838 - protein, 40 aa, synthetic construct; identical to SEQ ID NO: 836

SEQ ID NO: 839 - protein, 27 aa, synthetic construct
DWGTAIGNIGNNAAANWATGGNAGWNK

SEQ ID NO: 840 - protein, 38 aa, synthetic construct; identical to SEQ ID NO: 834

SEQ ID NO: 841 - protein, 42 aa, synthetic construct
TNWGSVVGSCVAGGLVGALGGTPISIGAGCLVGAGQDWISQK

SEQ ID NO: 842 - protein, 31 aa, synthetic construct
LGKLYKWITNVIDNIFYTPKGIPCPDDVYRG

SEQ ID NO: 843 - protein, 17 aa, synthetic construct
GYIGGGNHLCKGSAARF

SEQ ID NO: 844 - protein, 20 aa, synthetic construct
AGLGFASSFASVMSCNGVYL

SEQ ID NO: 845 - protein, 20 aa, synthetic construct
IVNGWISSLGGGYFHPGYRG

SEQ ID NO: 846 - protein, 27 aa, synthetic construct
DWGTAIGNIGNNAAANWATGGNAGWNK

SEQ ID NO: 847 - protein, 27 aa, synthetic construct
GAYGAGYAVGKAYAHYKNSQEVPVVAE

SEQ ID NO: 848 - protein, 11 aa, synthetic construct
TAGAAYSGCFD

SEQ ID NO: 849 - protein, 15 aa, synthetic construct
GWLAGAAGVINSCMK

SEQ ID NO: 850 - protein, 21 aa, synthetic construct
GGLGILGGVGGAIYGASESCF

SEQ ID NO: 851 - protein, 22 aa, synthetic construct
QLLGSICGFIDGISKSTNRGQC

SEQ ID NO: 852 - protein, 21 aa, synthetic construct
GGLGILGGVGGAIYGASESCF

SEQ ID NO: 853 - protein, 21 aa, synthetic construct
GGLGILGGVGGAIYGASESCF

SEQ ID NO: 854 - protein, 21 aa, synthetic construct
GGLGILGGVGGAIYGASESCF

SEQ ID NO: 855 - protein, 12 aa, synthetic construct
GDGGGGTAVGHF

SEQ ID NO: 856 - protein, 12 aa, synthetic construct
GDGGGGTAVGHF

SEQ ID NO: 857 - protein, 12 aa, synthetic construct
GDGGGGTAVGHF

SEQ ID NO: 858 - protein, 22 aa, synthetic construct
QGFGAICSFIDGFNGKKYRGKC

SEQ ID NO: 859 - protein, 22 aa, synthetic construct
QGFGAICSFIDGFNGKKYRGKC

SEQ ID NO: 860 - protein, 36 aa, synthetic construct
QRVHAACNELLRGQRAAVEDRSCGDPVELSGSGRRP

SEQ ID NO: 861 - protein, 38 aa, synthetic construct
GCASRGVKLGSRIGPWGAVIGGVGSAAVCGYLAYKASS

SEQ ID NO: 862 - protein, 38 aa, synthetic construct; identical to SEQ ID NO: 861

SEQ ID NO: 863 - protein, 38 aa, synthetic construct; identical to SEQ ID NO: 861

SEQ ID NO: 864 - protein, 38 aa, synthetic construct; identical to SEQ ID NO: 861

SEQ ID NO: 865 - protein, 38 aa, synthetic construct; identical to SEQ ID NO: 861

SEQ ID NO: 866 - protein, 38 aa, synthetic construct; identical to SEQ ID NO: 861

SEQ ID NO: 867 - protein, 23 aa, synthetic construct
WGAAIGGIGGAVVCGYLAYTATS

SEQ ID NO: 868 - protein, 37 aa, synthetic construct
DQLKELYGGGAHANNMSCITGSKSCPNCKEAEREDYN

SEQ ID NO: 869 - protein, 16 aa, synthetic construct
IDDMQKAVEELDAIKC

SEQ ID NO: 870 - protein, 39 aa, synthetic construct
AQGSSIGENYNANVSNGYTESGGGNVACGDGVPATKPTH

SEQ ID NO: 871 - protein, 39 aa, synthetic construct; identical to SEQ ID NO: 870

SEQ ID NO: 872 - protein, 39 aa, synthetic construct; identical to SEQ ID NO: 870

SEQ ID NO: 873 - protein, 27 aa, synthetic construct
ARGAKEAVDRIRISQEVSSDEVAIVCY

SEQ ID NO: 874 - protein, 21 aa, synthetic construct
GHGIGSLAGACINGVCAGDVI

SEQ ID NO: 875 - protein, 15 aa, synthetic construct
MRACYNDYCGASCKK

SEQ ID NO: 876 - protein, 32 aa, synthetic construct
GGCRSIAGAGPDTYLCSPPTSGPKIEPFPIEW

SEQ ID NO: 877 - protein, 32 aa, synthetic construct; identical to SEQ ID NO: 876

SEQ ID NO: 878 - protein, 27 aa, synthetic construct
ADDARHAFEKARDAELHRGFQILSHGD

SEQ ID NO: 879 - protein, 27 aa, synthetic construct
ADEARHAFEKARDAELHRGFQILSHGD

SEQ ID NO: 880 - protein, 27 aa, synthetic construct
ADEARHAYDKARDAELHRGFQILSHGD

SEQ ID NO: 881 - protein, 27 aa, synthetic construct
ADEARHAFEKARDAELHRGFQILSHGD

SEQ ID NO: 882 - protein, 27 aa, synthetic construct
ADDARHAFEKARDAELHRGFQILSHGD

SEQ ID NO: 883 - protein, 28 aa, synthetic construct
HGATTGWSDLGDGIGSMWGGIWNGLNGH

SEQ ID NO: 884 - protein, 26 aa, synthetic construct
SEARAAFDKARDAELHRGFAVLHCSP

SEQ ID NO: 885 - protein, 15 aa, synthetic construct
SWIDDFMKGFKKTFC

SEQ ID NO: 886 - protein, 15 aa, synthetic construct
SWIDDFMKGFKKTFC

SEQ ID NO: 887 - protein, 15 aa, synthetic construct
SWIDDFMKGFKKTFC

SEQ ID NO: 888 - protein, 33 aa, synthetic construct
KDLITVIGGAAAEKPKHQVTVKPDGTIVIVVYR

SEQ ID NO: 889 - protein, 19 aa, synthetic construct
LDSVKGFLDGLTGHKRHYD

SEQ ID NO: 890 - protein, 39 aa, synthetic construct
AAGASCGSSGSSKDCDGTAVCNCRCPIIVHKPVEKPSHE

SEQ ID NO: 891 - protein, 19 aa, synthetic construct
LMGTFAGAFDEYARKRGKH

SEQ ID NO: 892 - protein, 35 aa, synthetic construct
EEYCENLRKIIANNKLSEKALAGWTIGYSTHCVEH

SEQ ID NO: 893 - protein, 36 aa, synthetic construct
LKEYASCAEDAKVKQFFEQGAKSAMENKQQLMKFLQ

SEQ ID NO: 894 - protein, 29 aa, synthetic construct
KPVTGFGEQVITWQDHKLVHKKVSATERL

SEQ ID NO: 895 - protein, 26 aa, synthetic construct
SSVKQFFEKGARSAMDNKQQLMKFLQ

SEQ ID NO: 896 - protein, 11 aa, synthetic construct
LEGYGAKLCEC

SEQ ID NO: 897 - protein, 16 aa, synthetic construct
NGLTGGNAGWHSGGII

SEQ ID NO: 898 - protein, 18 aa, synthetic construct
EAFSAGVHRLANGGNGFW

SEQ ID NO: 899 - protein, 37 aa, synthetic construct
NRLVQVVGGWLHSFALVTYFCMLLGSHSFAAYLQPEL

SEQ ID NO: 900 - protein, 16 aa, synthetic construct
NGLTGGNAGWHSGGII

SEQ ID NO: 901 - protein, 37 aa, synthetic construct
EFGRAAGQTLREFKNATNGMMKEHDETDKKEIEKKEQ

SEQ ID NO: 902 - protein, 37 aa, synthetic construct
EFGRAAGQTLREFKNATNGMMKEHDENDKKEIEKKEQ

SEQ ID NO: 903 - protein, 31 aa, synthetic construct
DFWEEMGKGFSKAGDDISDAWKKARDAVHGN

SEQ ID NO: 904 - protein, 18 aa, synthetic construct
LMGTFAGAFDEYARKRGK

SEQ ID NO: 905 - protein, 28 aa, synthetic construct
RAIDGAIEGATGKDIKTHARDFVRSLLD

SEQ ID NO: 906 - protein, 22 aa, synthetic construct
QFFEKGARSAMENKQQLMKFLQ

SEQ ID NO: 907 - protein, 38 aa, synthetic construct
NGVDGGLGVLSVAGSTKKKRPVEMRDLPPRKIPFFTEA

SEQ ID NO: 908 - protein, 30 aa, synthetic construct
TGGGWWEKLLHETILGKFKITKALELPIQL

SEQ ID NO: 909 - protein, 19 aa, synthetic construct
RAIDGAIEGATGKDIKTHA

SEQ ID NO: 910 - protein, 21 aa, synthetic construct
TLLTGVFGWLKKFQEKPQKHM

SEQ ID NO: 911 - protein, 15 aa, synthetic construct
KLMDSLCRNFNSKKC

SEQ ID NO: 912 - protein, 19 aa, synthetic construct
LMGTFAGAFDEYARKRGKY

SEQ ID NO: 913 - protein, 22 aa, synthetic construct
LRKVVDAIGSIVNLIPFFGKRA

SEQ ID NO: 914 - protein, 39 aa, synthetic construct
ETLAQGIKRVYDSIWPNDTAWYTGKNNKTNIPPYSPYGH

SEQ ID NO: 915 - protein, 34 aa, synthetic construct
AVGAGAGGVIGAIGGLLTECVQWGYNETPQHGWN

SEQ ID NO: 916 - protein, 27 aa, synthetic construct
GGGWLEDLFSPYLKKYKLGKLGQPDLG

SEQ ID NO: 917 - protein, 27 aa, synthetic construct
GGGWLEDLFSPYLKKYKLGKLGQPDLG

SEQ ID NO: 918 - protein, 42 aa, synthetic construct
AGLGNGLKQWNTTGFSEYNIGGGKVNNGKAPTSIPAYNPYGH

SEQ ID NO: 919 - protein, 29 aa, synthetic construct
AAIAKGAAAIGTAYMAGYTLGTHMKKKNP

SEQ ID NO: 920 - protein, 29 aa, synthetic construct
AAIAKGAAAIGTAYMAGYTLGTHMKKKNP

SEQ ID NO: 921 - protein, 33 aa, synthetic construct
SGGGKGVNPASAAPITPMADSLFPVKKEEGTTP

SEQ ID NO: 922 - protein, 29 aa, synthetic construct
AAIAKGAAAIGTAYMAGYTLGTHMKKKNP

SEQ ID NO: 923 - protein, 29 aa, synthetic construct
AAIAKGAAAIGTAYMAGYTLGTHMKKKNP

SEQ ID NO: 924 - protein, 20 aa, synthetic construct
HRLRALFGGGEEGNTLEQTD

SEQ ID NO: 925 - protein, 32 aa, synthetic construct
LAATWGATFGGMLAPISLIGSVPGVLAACSGL

SEQ ID NO: 926 - protein, 32 aa, synthetic construct; identical to SEQ ID NO: 925

SEQ ID NO: 927 - protein, 21 aa, synthetic construct
IIEGGIESIKRLVQVTNLIII

SEQ ID NO: 928 - protein, 32 aa, synthetic construct; identical to SEQ ID NO: 925

SEQ ID NO: 929 - protein, 26 aa, synthetic construct
AITKGMAWGAGALAAGIGTGYLINKH

SEQ ID NO: 930 - protein, 23 aa, synthetic construct
WGAVIGGVGSAAVCGYLAYKASS

SEQ ID NO: 931 - protein, 40 aa, synthetic construct
TAAFTGLMTFAGVSGPVGWVLGTFVGGAWLGASAAAGCLK

SEQ ID NO: 932 - protein, 23 aa, synthetic construct
WGAVIGGVGSAAVCGYLAYKASS

SEQ ID NO: 933 - protein, 12 aa, synthetic construct
DFGRGFVDGFRG

SEQ ID NO: 934 - protein, 12 aa, synthetic construct
DFGRGFVDGFRG

SEQ ID NO: 935 - protein, 35 aa, synthetic construct
FTALLSLLGGAIYVTFQITWTVSHSDDKNKKNDRL

SEQ ID NO: 936 - protein, 16 aa, synthetic construct
NGLTGGNAGWHSGGII

SEQ ID NO: 937 - protein, 42 aa, synthetic construct
VIGGLGGLASGLKFCKLPHPVLTGGCVVGFTVGGAYLGYTAN

SEQ ID NO: 938 - protein, 42 aa, synthetic construct
VIGGLGGLASGLKFCKLPHPVLAGGCVVGFTVGGAYLGYTAN

SEQ ID NO: 939 - protein, 42 aa, synthetic construct; identical to SEQ ID NO: 937

SEQ ID NO: 940 - protein, 42 aa, synthetic construct; identical to SEQ ID NO: 937

SEQ ID NO: 941 - protein, 42 aa, synthetic construct; identical to SEQ ID NO: 938

SEQ ID NO: 942 - protein, 42 aa, synthetic construct; identical to SEQ ID NO: 937

SEQ ID NO: 943 - protein, 42 aa, synthetic construct; identical to SEQ ID NO: 937

SEQ ID NO: 944 - protein, 42 aa, synthetic construct; identical to SEQ ID NO: 938

SEQ ID NO: 945 - protein, 42 aa, synthetic construct
VIGGLGGLASGLKFCKLLHPVLAGGCVVGFTVGGAYLGYTAN

SEQ ID NO: 946 - protein, 42 aa, synthetic construct; identical to SEQ ID NO: 945

SEQ ID NO: 947 - protein, 42 aa, synthetic construct; identical to SEQ ID NO: 938

SEQ ID NO: 948 - protein, 42 aa, synthetic construct; identical to SEQ ID NO: 937

SEQ ID NO: 949 - protein, 42 aa, synthetic construct; identical to SEQ ID NO: 937

SEQ ID NO: 950 - protein, 42 aa, synthetic construct; identical to SEQ ID NO: 937

SEQ ID NO: 951 - protein, 42 aa, synthetic construct; identical to SEQ ID NO: 937

SEQ ID NO: 952 - protein, 20 aa, synthetic construct
GGALLGLAGGAVSYGALCWV

SEQ ID NO: 953 - protein, 20 aa, synthetic construct
GGALLGLAGGAVSYGALCWV

SEQ ID NO: 954 - protein, 36 aa, synthetic construct
DAGHYAGKAVQALGVGIGIAAGIATLFVAKNTNGDK

SEQ ID NO: 955 - protein, 38 aa, synthetic construct
KKLETYAQNCADPELKAILQRDAQAAKQSQQQLMTFLG

SEQ ID NO: 956 - protein, 27 aa, synthetic construct
GGGWLEDLFSPYLKKYKLGKLGQHDLG

SEQ ID NO: 957 - protein, 12 aa, synthetic construct
QFCQSGIHFLST

SEQ ID NO: 958 - protein, 42 aa, synthetic construct
ATVANKLDQYAHQCTDPQLKQMLQQDAQAARNTKQQLMSFLG

SEQ ID NO: 959 - protein, 36 aa, synthetic construct
GGGRGGRDGRGGRGGRGGRGGRGGRGGYYGGYYYDD

SEQ ID NO: 960 - protein, 32 aa, synthetic construct
GGKAVNGFVNGNFSSRISLFCTFTKNDSKINE

SEQ ID NO: 961 - protein, 32 aa, synthetic construct; identical to SEQ ID NO: 960

SEQ ID NO: 962 - protein, 21 aa, synthetic construct
QIGESLGNYIRRKTENKMCDI

SEQ ID NO: 963 - protein, 21 aa, synthetic construct
NIGEAFGSYIRRKSEGKMCDF

SEQ ID NO: 964 - protein, 18 aa, synthetic construct
VGAVYTAGYTLGKMNGRR

SEQ ID NO: 965 - protein, 30 aa, synthetic construct
ELMRGLGSGIKEFKDASKDDDSNDKLEEKK

SEQ ID NO: 966 - protein, 30 aa, synthetic construct
RGGESIATNCAACEGCQPNAKETDVPSQAQ

SEQ ID NO: 967 - protein, 13 aa, synthetic construct
AQEFQQMMNKFPQ

SEQ ID NO: 968 - protein, 41 aa, synthetic construct
KYYTAICATYQAALDLCARVGVDPSTIDVTDNPFPSWGFGA

SEQ ID NO: 969 - protein, 17 aa, synthetic construct
SVFNAGYKFGADLARRR

SEQ ID NO: 970 - protein, 37 aa, synthetic construct
ELMNTAGGSITIATVGAGLAIFLGGRLIGQDLKRKFG

SEQ ID NO: 971 - protein, 42 aa, synthetic construct
GGFSAGFTAGAGIGAAVGGPMAPATALIGGMTGEGVRKSVSA

SEQ ID NO: 972 - protein, 38 aa, synthetic construct
GAAAGALGGSAAFGIGAFGGAILGVNVGAAAGAIRCLA

SEQ ID NO: 973 - protein, 41 aa, synthetic construct
WGKVGAAGVGCGLITWWTGPGAVADGALCAATQFFGEVLKN

SEQ ID NO: 974 - protein, 41 aa, synthetic construct; identical to SEQ ID NO: 973

SEQ ID NO: 975 - protein, 41 aa, synthetic construct; identical to SEQ ID NO: 973

SEQ ID NO: 976 - protein, 25 aa, synthetic construct
AAYGAAATVAGGTLGLGVYNGYRGR

SEQ ID NO: 977 - protein, 25 aa, synthetic construct
AAYGAAATLAGGTLGLGVYNGYRGR

SEQ ID NO: 978 - protein, 25 aa, synthetic construct
AAYGAAATVAGGTLGLGVYNGYRGR

SEQ ID NO: 979 - protein, 19 aa, synthetic construct
VASFAAIISGIATYNAIRK

SEQ ID NO: 980 - protein, 36 aa, synthetic construct
MQEYAECAQDAKVKQFFEKGAKSAMENKQALMDFLR

SEQ ID NO: 981 - protein, 22 aa, synthetic construct
QFFEKGARSAMENKQQLMKFLQ

SEQ ID NO: 982 - protein, 17 aa, synthetic construct
IANGWAGSAAGGYFSGR

SEQ ID NO: 983 - protein, 21 aa, synthetic construct
KLFQEAADSAMKNKQELMKFL

SEQ ID NO: 984 - protein, 21 aa, synthetic construct
KLFQEAADSAMKNKQELMKFL

SEQ ID NO: 985 - protein, 22 aa, synthetic construct
QFFEQGAKSAMENKQQLMKFLQ

SEQ ID NO: 986 - protein, 21 aa, synthetic construct
KLFQEAADSAMKNKQELMKFL

SEQ ID NO: 987 - protein, 40 aa, synthetic construct
NLGSAIGGCIGGVILAAATGPLTGGAAALVCVGSGLSAYL

SEQ ID NO: 988 - protein, 36 aa, synthetic construct
DGVKAAVNKLAHTPYERTVTRHGEKYTVKYDGNPGW

SEQ ID NO: 989 - protein, 21 aa, synthetic construct
RFVQAWADSVDGEKPISLPPQ

SEQ ID NO: 990 - protein, 24 aa, synthetic construct
LIDAGIGVGSAIRRVHDDKLCPLE

SEQ ID NO: 991 - protein, 42 aa, synthetic construct
ATIINKLEQYAQACTDPKLKQMLEKDAQDARNTKQQLMTFLG

SEQ ID NO: 992 - protein, 42 aa, synthetic construct; identical to SEQ ID NO: 991

SEQ ID NO: 993 - protein, 21 aa, synthetic construct
KLFQDAANSAMKNKQDLMKFL

SEQ ID NO: 994 - protein, 42 aa, synthetic construct
LEQIVGGGIEGFGIYIGGATQVKNSEASLIFRLLHHKRHGRN

SEQ ID NO: 995 - protein, 17 aa, synthetic construct
GAIAGGADEYARKRGHH

SEQ ID NO: 996 - protein, 42 aa, synthetic construct
ATIINKLEQYAQTCTDPQLKQMLQKDAQDARNTKQQLMTFLG

SEQ ID NO: 997 - protein, 42 aa, synthetic construct; identical to SEQ ID NO: 996

SEQ ID NO: 998 - protein, 42 aa, synthetic construct; identical to SEQ ID NO: 996

SEQ ID NO: 999 - protein, 18 aa, synthetic construct
GVISNGWGSAGAGFGPQH

SEQ ID NO: 1000 - protein, 42 aa, synthetic construct; identical to SEQ ID NO: 996

SEQ ID NO: 1001 - protein, 42 aa, synthetic construct; identical to SEQ ID NO: 996

SEQ ID NO: 1002 - protein, 42 aa, synthetic construct; identical to SEQ ID NO: 996

SEQ ID NO: 1003 - protein, 40 aa, synthetic construct
SGIHNGADPAAHTVAVPAADSLVMAPFNSDSNDDKGITGK

SEQ ID NO: 1004 - protein, 42 aa, synthetic construct
ATIISKLEQYAQTCTDPQLKQMLQKDAQDARNTKQQLMTFLG

SEQ ID NO: 1005 - protein, 42 aa, synthetic construct; identical to SEQ ID NO: 996

SEQ ID NO: 1006 - protein, 42 aa, synthetic construct; identical to SEQ ID NO: 996

SEQ ID NO: 1007 - protein, 42 aa, synthetic construct; identical to SEQ ID NO: 996

SEQ ID NO: 1008 - protein, 42 aa, synthetic construct; identical to SEQ ID NO: 996

SEQ ID NO: 1009 - protein, 42 aa, synthetic construct; identical to SEQ ID NO: 996

SEQ ID NO: 1010 - protein, 42 aa, synthetic construct
ATIINKLEQYAQACTDPQLKQMLQKDAQDARNTKQQLMTFLG

SEQ ID NO: 1011 - protein, 42 aa, synthetic construct; identical to SEQ ID NO: 1010

SEQ ID NO: 1012 - protein, 42 aa, synthetic construct
ATIINKLEQYAQTCTDPQLKQMLQKDAQDARNTKQQLLTFLG

SEQ ID NO: 1013 - protein, 42 aa, synthetic construct; identical to SEQ ID NO: 996

SEQ ID NO: 1014 - protein, 42 aa, synthetic construct; identical to SEQ ID NO: 996

SEQ ID NO: 1015 - protein, 42 aa, synthetic construct; identical to SEQ ID NO: 996

SEQ ID NO: 1016 - protein, 42 aa, synthetic construct; identical to SEQ ID NO: 996

SEQ ID NO: 1017 - protein, 42 aa, synthetic construct; identical to SEQ ID NO: 996

SEQ ID NO: 1018 - protein, 42 aa, synthetic construct; identical to SEQ ID NO: 996

SEQ ID NO: 1019 - protein, 42 aa, synthetic construct; identical to SEQ ID NO: 996

SEQ ID NO: 1020 - protein, 42 aa, synthetic construct; identical to SEQ ID NO: 996

SEQ ID NO: 1021 - protein, 42 aa, synthetic construct; identical to SEQ ID NO: 996

SEQ ID NO: 1022 - protein, 42 aa, synthetic construct; identical to SEQ ID NO: 996

SEQ ID NO: 1023 - protein, 42 aa, synthetic construct; identical to SEQ ID NO: 996

SEQ ID NO: 1024 - protein, 42 aa, synthetic construct; identical to SEQ ID NO: 996

SEQ ID NO: 1025 - protein, 42 aa, synthetic construct; identical to SEQ ID NO: 996

SEQ ID NO: 1026 - protein, 42 aa, synthetic construct; identical to SEQ ID NO: 996

SEQ ID NO: 1027 - protein, 42 aa, synthetic construct; identical to SEQ ID NO: 996

SEQ ID NO: 1028 - protein, 42 aa, synthetic construct; identical to SEQ ID NO: 996

SEQ ID NO: 1029 - protein, 42 aa, synthetic construct; identical to SEQ ID NO: 996

SEQ ID NO: 1030 - protein, 42 aa, synthetic construct; identical to SEQ ID NO: 996

SEQ ID NO: 1031 - protein, 42 aa, synthetic construct; identical to SEQ ID NO: 996

SEQ ID NO: 1032 - protein, 42 aa, synthetic construct; identical to SEQ ID NO: 996

SEQ ID NO: 1033 - protein, 42 aa, synthetic construct; identical to SEQ ID NO: 996

SEQ ID NO: 1034 - protein, 42 aa, synthetic construct; identical to SEQ ID NO: 996

SEQ ID NO: 1035 - protein, 42 aa, synthetic construct; identical to SEQ ID NO: 1010

SEQ ID NO: 1036 - protein, 42 aa, synthetic construct; identical to SEQ ID NO: 996

SEQ ID NO: 1037 - protein, 42 aa, synthetic construct; identical to SEQ ID NO: 996

SEQ ID NO: 1038 - protein, 42 aa, synthetic construct; identical to SEQ ID NO: 996

SEQ ID NO: 1039 - protein, 42 aa, synthetic construct; identical to SEQ ID NO: 996

SEQ ID NO: 1040 - protein, 42 aa, synthetic construct; identical to SEQ ID NO: 996

SEQ ID NO: 1041 - protein, 42 aa, synthetic construct; identical to SEQ ID NO: 996

SEQ ID NO: 1042 - protein, 42 aa, synthetic construct; identical to SEQ ID NO: 996

SEQ ID NO: 1043 - protein, 42 aa, synthetic construct; identical to SEQ ID NO: 996

SEQ ID NO: 1044 - protein, 42 aa, synthetic construct; identical to SEQ ID NO: 996

SEQ ID NO: 1045 - protein, 42 aa, synthetic construct; identical to SEQ ID NO: 996

SEQ ID NO: 1046 - protein, 42 aa, synthetic construct; identical to SEQ ID NO: 996

SEQ ID NO: 1047 - protein, 42 aa, synthetic construct; identical to SEQ ID NO: 996

SEQ ID NO: 1048 - protein, 42 aa, synthetic construct; identical to SEQ ID NO: 996

SEQ ID NO: 1049 - protein, 42 aa, synthetic construct; identical to SEQ ID NO: 996

SEQ ID NO: 1050 - protein, 42 aa, synthetic construct; identical to SEQ ID NO: 1010

SEQ ID NO: 1051 - protein, 42 aa, synthetic construct; identical to SEQ ID NO: 996

SEQ ID NO: 1052 - protein, 42 aa, synthetic construct; identical to SEQ ID NO: 1010

SEQ ID NO: 1053 - protein, 42 aa, synthetic construct; identical to SEQ ID NO: 996

SEQ ID NO: 1054 - protein, 42 aa, synthetic construct; identical to SEQ ID NO: 996

SEQ ID NO: 1055 - protein, 42 aa, synthetic construct; identical to SEQ ID NO: 996

SEQ ID NO: 1056 - protein, 42 aa, synthetic construct; identical to SEQ ID NO: 996

SEQ ID NO: 1057 - protein, 42 aa, synthetic construct; identical to SEQ ID NO: 996

SEQ ID NO: 1058 - protein, 42 aa, synthetic construct; identical to SEQ ID NO: 996

SEQ ID NO: 1059 - protein, 42 aa, synthetic construct; identical to SEQ ID NO: 996

SEQ ID NO: 1060 - protein, 42 aa, synthetic construct; identical to SEQ ID NO: 996

SEQ ID NO: 1061 - protein, 42 aa, synthetic construct; identical to SEQ ID NO: 996

SEQ ID NO: 1062 - protein, 42 aa, synthetic construct; identical to SEQ ID NO: 996

SEQ ID NO: 1063 - protein, 42 aa, synthetic construct; identical to SEQ ID NO: 996

SEQ ID NO: 1064 - protein, 42 aa, synthetic construct; identical to SEQ ID NO: 996

SEQ ID NO: 1065 - protein, 42 aa, synthetic construct; identical to SEQ ID NO: 996

SEQ ID NO: 1066 - protein, 42 aa, synthetic construct; identical to SEQ ID NO: 996

SEQ ID NO: 1067 - protein, 42 aa, synthetic construct; identical to SEQ ID NO: 1010

SEQ ID NO: 1068 - protein, 42 aa, synthetic construct; identical to SEQ ID NO: 996

SEQ ID NO: 1069 - protein, 42 aa, synthetic construct; identical to SEQ ID NO: 996

SEQ ID NO: 1070 - protein, 42 aa, synthetic construct; identical to SEQ ID NO: 996

SEQ ID NO: 1071 - protein, 42 aa, synthetic construct; identical to SEQ ID NO: 996

SEQ ID NO: 1072 - protein, 42 aa, synthetic construct; identical to SEQ ID NO: 996

SEQ ID NO: 1073 - protein, 42 aa, synthetic construct; identical to SEQ ID NO: 996

SEQ ID NO: 1074 - protein, 42 aa, synthetic construct; identical to SEQ ID NO: 996

SEQ ID NO: 1075 - protein, 42 aa, synthetic construct; identical to SEQ ID NO: 996

SEQ ID NO: 1076 - protein, 42 aa, synthetic construct; identical to SEQ ID NO: 1012

SEQ ID NO: 1077 - protein, 42 aa, synthetic construct; identical to SEQ ID NO: 996

SEQ ID NO: 1078 - protein, 42 aa, synthetic construct; identical to SEQ ID NO: 996

SEQ ID NO: 1079 - protein, 42 aa, synthetic construct; identical to SEQ ID NO: 996

SEQ ID NO: 1080 - protein, 42 aa, synthetic construct; identical to SEQ ID NO: 996

SEQ ID NO: 1081 - protein, 42 aa, synthetic construct; identical to SEQ ID NO: 996

SEQ ID NO: 1082 - protein, 42 aa, synthetic construct; identical to SEQ ID NO: 1010

SEQ ID NO: 1083 - protein, 42 aa, synthetic construct; identical to SEQ ID NO: 996

SEQ ID NO: 1084 - protein, 42 aa, synthetic construct; identical to SEQ ID NO: 996

SEQ ID NO: 1085 - protein, 42 aa, synthetic construct; identical to SEQ ID NO: 996

SEQ ID NO: 1086 - protein, 42 aa, synthetic construct; identical to SEQ ID NO: 996

SEQ ID NO: 1087 - protein, 42 aa, synthetic construct; identical to SEQ ID NO: 996

SEQ ID NO: 1088 - protein, 42 aa, synthetic construct; identical to SEQ ID NO: 996

SEQ ID NO: 1089 - protein, 42 aa, synthetic construct; identical to SEQ ID NO: 996

SEQ ID NO: 1090 - protein, 42 aa, synthetic construct; identical to SEQ ID NO: 1010

SEQ ID NO: 1091 - protein, 42 aa, synthetic construct; identical to SEQ ID NO: 996

SEQ ID NO: 1092 - protein, 42 aa, synthetic construct; identical to SEQ ID NO: 996

SEQ ID NO: 1093 - protein, 42 aa, synthetic construct; identical to SEQ ID NO: 996

SEQ ID NO: 1094 - protein, 42 aa, synthetic construct; identical to SEQ ID NO: 996

SEQ ID NO: 1095 - protein, 42 aa, synthetic construct; identical to SEQ ID NO: 996

SEQ ID NO: 1096 - protein, 42 aa, synthetic construct; identical to SEQ ID NO: 996

SEQ ID NO: 1097 - protein, 42 aa, synthetic construct; identical to SEQ ID NO: 996

SEQ ID NO: 1098 - protein, 42 aa, synthetic construct; identical to SEQ ID NO: 996

SEQ ID NO: 1099 - protein, 42 aa, synthetic construct; identical to SEQ ID NO: 996

SEQ ID NO: 1100 - protein, 42 aa, synthetic construct; identical to SEQ ID NO: 1010

SEQ ID NO: 1101 - protein, 42 aa, synthetic construct; identical to SEQ ID NO: 996

SEQ ID NO: 1102 - protein, 42 aa, synthetic construct; identical to SEQ ID NO: 1010

SEQ ID NO: 1103 - protein, 42 aa, synthetic construct; identical to SEQ ID NO: 996

SEQ ID NO: 1104 - protein, 42 aa, synthetic construct; identical to SEQ ID NO: 996

SEQ ID NO: 1105 - protein, 20 aa, synthetic construct
AGAAGGAIVGGVGYGATCWW

SEQ ID NO: 1106 - protein, 20 aa, synthetic construct
AGAAGGAIVGGVGYGATCWW

SEQ ID NO: 1107 - protein, 20 aa, synthetic construct
AGAAGGAIVGGVGYGATCWW

SEQ ID NO: 1108 - protein, 20 aa, synthetic construct
AGAAGGAIVGGVGYGATCWW

SEQ ID NO: 1109 - protein, 20 aa, synthetic construct
AGAAGGAIVGGVGYGATCWW

SEQ ID NO: 1110 - protein, 20 aa, synthetic construct
AGAAGGAIVGGVGYGATCWW

SEQ ID NO: 1111 - protein, 20 aa, synthetic construct
AGAAGGAIVGGVGYGATCWW

SEQ ID NO: 1112 - protein, 20 aa, synthetic construct
AGAAGGAIVGGVGYGATCWW

SEQ ID NO: 1113 - protein, 20 aa, synthetic construct
AGAAGGAIVGGVGYGATCWW

SEQ ID NO: 1114 - protein, 42 aa, synthetic construct
ALSGAYQAGQQCILAGPQAYLICAAGGAVVGGLIAYGLRPPK

SEQ ID NO: 1115 - protein, 27 aa, synthetic construct
GGGWLEDLFSPYLKKYKLGKLGQPDLG

SEQ ID NO: 1116 - protein, 27 aa, synthetic construct
GGGWLEDLFSPYLKKYKLGKLGQPDLG

SEQ ID NO: 1117 - protein, 29 aa, synthetic construct
AVIGGALGGLGGAGGGCLTGAIGSIWDQW

SEQ ID NO: 1118 - protein, 33 aa, synthetic construct
AAMGGAIGAFGGPVGAIMGAWGGAVGGAMKYSI

SEQ ID NO: 1119 - protein, 20 aa, synthetic construct
AGAAGGAIVGGVGYGATCWW

SEQ ID NO: 1120 - protein, 20 aa, synthetic construct
AGAAGGAIVGGVGYGATCWW

SEQ ID NO: 1121 - protein, 20 aa, synthetic construct
AGAAGGAIVGGVGYGATCWW

SEQ ID NO: 1122 - protein, 20 aa, synthetic construct
AGAAGGAIVGGVGYGATCWW

SEQ ID NO: 1123 - protein, 33 aa, synthetic construct; identical to SEQ ID NO: 1118

SEQ ID NO: 1124 - protein, 13 aa, synthetic construct
AVAGGVYAGYKSR

SEQ ID NO: 1125 - protein, 29 aa, synthetic construct
AIIGGALGGLGGAGGGCLTGAIGSIWDQW

SEQ ID NO: 1126 - protein, 33 aa, synthetic construct; identical to SEQ ID NO: 1118

SEQ ID NO: 1127 - protein, 42 aa, synthetic construct
ATIINKLDQYAQACTDPQLKQMLQKDAQDARNTKQQLMTFLG

SEQ ID NO: 1128 - protein, 42 aa, synthetic construct; identical to SEQ ID NO: 1127

SEQ ID NO: 1129 - protein, 42 aa, synthetic construct; identical to SEQ ID NO: 1127

SEQ ID NO: 1130 - protein, 42 aa, synthetic construct; identical to SEQ ID NO: 1127

SEQ ID NO: 1131 - protein, 42 aa, synthetic construct; identical to SEQ ID NO: 1127

SEQ ID NO: 1132 - protein, 42 aa, synthetic construct; identical to SEQ ID NO: 1127

SEQ ID NO: 1133 - protein, 42 aa, synthetic construct; identical to SEQ ID NO: 1127

SEQ ID NO: 1134 - protein, 42 aa, synthetic construct; identical to SEQ ID NO: 1127

SEQ ID NO: 1135 - protein, 42 aa, synthetic construct; identical to SEQ ID NO: 1127

SEQ ID NO: 1136 - protein, 42 aa, synthetic construct; identical to SEQ ID NO: 1127

SEQ ID NO: 1137 - protein, 42 aa, synthetic construct; identical to SEQ ID NO: 1127

SEQ ID NO: 1138 - protein, 42 aa, synthetic construct; identical to SEQ ID NO: 1127

SEQ ID NO: 1139 - protein, 42 aa, synthetic construct; identical to SEQ ID NO: 1127

SEQ ID NO: 1140 - protein, 42 aa, synthetic construct; identical to SEQ ID NO: 1127

SEQ ID NO: 1141 - protein, 42 aa, synthetic construct; identical to SEQ ID NO: 1127

SEQ ID NO: 1142 - protein, 42 aa, synthetic construct; identical to SEQ ID NO: 1127

SEQ ID NO: 1143 - protein, 42 aa, synthetic construct; identical to SEQ ID NO: 1127

SEQ ID NO: 1144 - protein, 42 aa, synthetic construct; identical to SEQ ID NO: 1127

SEQ ID NO: 1145 - protein, 42 aa, synthetic construct; identical to SEQ ID NO: 1127

SEQ ID NO: 1146 - protein, 33 aa, synthetic construct
SAWGLMATGVGAPVGVAALGYILSTPSLAGCFL

SEQ ID NO: 1147 - protein, 21 aa, synthetic construct
QLFQDAANSAMKNKQDLMKFL

SEQ ID NO: 1148 - protein, 27 aa, synthetic construct
GGGWLEDLFSPYLKKYKLGKLGQPDLG

SEQ ID NO: 1149 - protein, 16 aa, synthetic construct
LIGLVDGYIRPLTCRG

SEQ ID NO: 1150 - protein, 15 aa, synthetic construct
LIGILDGIMRPLACN

SEQ ID NO: 1151 - protein, 16 aa, synthetic construct
SGAKTGWEIANKHKIY

SEQ ID NO: 1152 - protein, 16 aa, synthetic construct
SGAKTGWEIANKHKIY

SEQ ID NO: 1153 - protein, 16 aa, synthetic construct
SGAKTGWEIANKHKIY

SEQ ID NO: 1154 - protein, 41 aa, synthetic construct
AIAGGVGAGMATLGVGGVAGAFAGAIAGGLTCVGGMLFNGK

SEQ ID NO: 1155 - protein, 42 aa, synthetic construct
ATVANKLDQYAQQCTDPQLKQMLQQDAQAARNTKQQLMSFLG

SEQ ID NO: 1156 - protein, 42 aa, synthetic construct
GTIIHKLEQYAQNCTDPQLKSLLQKDIQDAQNTKQQLMSFLG

SEQ ID NO: 1157 - protein, 42 aa, synthetic construct
ATVANKLDQYAQQCTDPQLKQMLQQDAQAARNTRQQLMSFLG

SEQ ID NO: 1158 - protein, 42 aa, synthetic construct; identical to SEQ ID NO: 1155

SEQ ID NO: 1159 - protein, 42 aa, synthetic construct; identical to SEQ ID NO: 1155

SEQ ID NO: 1160 - protein, 42 aa, synthetic construct; identical to SEQ ID NO: 1155

SEQ ID NO: 1161 - protein, 42 aa, synthetic construct
TTIANKLDQYAQQCTDPQLKQILQQDAQAARNTKQQLMSFLG

SEQ ID NO: 1162 - protein, 42 aa, synthetic construct; identical to SEQ ID NO: 1157

SEQ ID NO: 1163 - protein, 42 aa, synthetic construct; identical to SEQ ID NO: 1157

SEQ ID NO: 1164 - protein, 21 aa, synthetic construct
QLFQEAADSALKNKQDLMKFL

SEQ ID NO: 1165 - protein, 21 aa, synthetic construct
KLFQDAADSAMKNKQDLMKFL

SEQ ID NO: 1166 - protein, 21 aa, synthetic construct
QLFQEAADSALKNKQDLMKFL

SEQ ID NO: 1167 - protein, 21 aa, synthetic construct
QLFQEAADSALKNKQDLMKFL

SEQ ID NO: 1168 - protein, 21 aa, synthetic construct
QLFQEAADSALKNKQDLMKFL

SEQ ID NO: 1169 - protein, 36 aa, synthetic construct
WGKVGAAGVGCGLITWWTGPGAVADGALCAATQFFG

SEQ ID NO: 1170 - protein, 37 aa, synthetic construct
KMTAYAQEAGDPAVKQFFQKSAQSAMQCKQQLMTFLQ

SEQ ID NO: 1171 - protein, 34 aa, synthetic construct
DFWRGVGKELGIIWAAAGDAGYCRAHKYGGPGHC

SEQ ID NO: 1172 - protein, 34 aa, synthetic construct; identical to SEQ ID NO: 1171

SEQ ID NO: 1173 - protein, 34 aa, synthetic construct
DFWRGVGKELSIIWAAAGDAGYCRAHKYGGPGHC

SEQ ID NO: 1174 - protein, 34 aa, synthetic construct; identical to SEQ ID NO: 1171

SEQ ID NO: 1175 - protein, 34 aa, synthetic construct; identical to SEQ ID NO: 1171

SEQ ID NO: 1176 - protein, 34 aa, synthetic construct; identical to SEQ ID NO: 1171

SEQ ID NO: 1177 - protein, 34 aa, synthetic construct; identical to SEQ ID NO: 1171

SEQ ID NO: 1178 - protein, 34 aa, synthetic construct; identical to SEQ ID NO: 1171

SEQ ID NO: 1179 - protein, 34 aa, synthetic construct; identical to SEQ ID NO: 1171

SEQ ID NO: 1180 - protein, 34 aa, synthetic construct; identical to SEQ ID NO: 1171

SEQ ID NO: 1181 - protein, 34 aa, synthetic construct; identical to SEQ ID NO: 1171

SEQ ID NO: 1182 - protein, 11 aa, synthetic construct
KVVKWVAGFFE

SEQ ID NO: 1183 - protein, 16 aa, synthetic construct
NGLTGGNAGWHSGGII

SEQ ID NO: 1184 - protein, 16 aa, synthetic construct
NGLTGGNAGWHSGGII

SEQ ID NO: 1185 - protein, 11 aa, synthetic construct
KVVKWVAGFFE

SEQ ID NO: 1186 - protein, 11 aa, synthetic construct
KVVKWVAGFFE

SEQ ID NO: 1187 - protein, 11 aa, synthetic construct
KVVKWVAGFFE

SEQ ID NO: 1188 - protein, 12 aa, synthetic construct
NGLTGGNAGWHN

SEQ ID NO: 1189 - protein, 11 aa, synthetic construct
KVVKWVAGFFE

SEQ ID NO: 1190 - protein, 11 aa, synthetic construct
KVVKWVAGFFE

SEQ ID NO: 1191 - protein, 17 aa, synthetic construct
GAVGGGLTGAATFCQEK

SEQ ID NO: 1192 - protein, 24 aa, synthetic construct
AKQCLQAIGSWGIAGTQGVPLELS

SEQ ID NO: 1193 - protein, 29 aa, synthetic construct
WALLASLASAVVFFIGAFTGYTNPTKCNN

SEQ ID NO: 1194 - protein, 17 aa, synthetic construct
AFVSGILNGIANPKKCN

SEQ ID NO: 1195 - protein, 37 aa, synthetic construct
LHNINGGISAAGILGGIAIAITFIASVIYGYFYPKEN

SEQ ID NO: 1196 - protein, 38 aa, synthetic construct
FLGAFAGAGGLICAAWGGGWMVGKFIHHRLVSDYPTIF

SEQ ID NO: 1197 - protein, 32 aa, synthetic construct
LEQVSGGGAAVVVGGIIVVAAIIWWASAQKAH

SEQ ID NO: 1198 - protein, 33 aa, synthetic construct
EDVLNLIAGGLAIAIFIGGILMMFSTVWTTKKK

SEQ ID NO: 1199 - protein, 18 aa, synthetic construct
GAIGTAVGAAAGAIIACR

SEQ ID NO: 1200 - protein, 23 aa, synthetic construct
WGAAIGGIGGAVVCGYLAYTATS

SEQ ID NO: 1201 - protein, 23 aa, synthetic construct
WGAAIGGIGGAVVCGYLAYTATS

SEQ ID NO: 1202 - protein, 23 aa, synthetic construct
WGAAIGGIGGAVVCGYLAYTATS

SEQ ID NO: 1203 - protein, 23 aa, synthetic construct
WGAAIGGIGGAVVCGYLAYTATS

SEQ ID NO: 1204 - protein, 27 aa, synthetic construct
YGKILGPWGAAIGGAVVCGYLAYTATS

SEQ ID NO: 1205 - protein, 25 aa, synthetic construct
GLWGAAIGGIGGAVVCGYLAYTATS

SEQ ID NO: 1206 - protein, 23 aa, synthetic construct
WGAAIGGIGGAVVCGYLAYTATS

SEQ ID NO: 1207 - protein, 23 aa, synthetic construct
WGAAIGGIGGAVVCGYLAYTATS

SEQ ID NO: 1208 - protein, 23 aa, synthetic construct
WGAAIGGIGGAVVCGYLAYTATS

SEQ ID NO: 1209 - protein, 23 aa, synthetic construct
WGAAIGGIGGAVVCGYLAYTATS

SEQ ID NO: 1210 - protein, 23 aa, synthetic construct
WGAAIGGIGGAVVCGYLAYTATS

SEQ ID NO: 1211 - protein, 25 aa, synthetic construct
GLWGAAIGGIGGAVVCGYLAYTATS

SEQ ID NO: 1212 - protein, 23 aa, synthetic construct
WGAAIGGIGGAVVCGYLAYTATS

SEQ ID NO: 1213 - protein, 23 aa, synthetic construct
WGAAIGGIGGAVVCGYLAYTATS

SEQ ID NO: 1214 - protein, 23 aa, synthetic construct
WGAAIGGIGGAVVCGYLAYTATS

SEQ ID NO: 1215 - protein, 19 aa, synthetic construct
WDKVGAGLGAVGIGLALYY

SEQ ID NO: 1216 - protein, 16 aa, synthetic construct
VAGGLVGAGQDWISQK

SEQ ID NO: 1217 - protein, 40 aa, synthetic construct
VAKKLETYAQNCADPELKEILQRDAQAAKQSQQQLMTFLG

SEQ ID NO: 1218 - protein, 12 aa, synthetic construct
GGYTHGGGGGRH

SEQ ID NO: 1219 - protein, 24 aa, synthetic construct
LSLLTGGLSDLIEGCINNQIRENQ

SEQ ID NO: 1220 - protein, 38 aa, synthetic construct
SFFGRAWKWIKEHVGIFDYGETLETSGSAIGVKVGVDL

SEQ ID NO: 1221 - protein, 21 aa, synthetic construct
EFGKGFLNSFAASHNNMEGDN

SEQ ID NO: 1222 - protein, 18 aa, synthetic construct
QICREVYGTGGHCTAATP

SEQ ID NO: 1223 - protein, 36 aa, synthetic construct
CEDGAQFGGSGNCPIDASLECANNGGAAYSVETGCS

SEQ ID NO: 1224 - protein, 22 aa, synthetic construct
LMGTVYTFIEKAPFPRLGWTLL

SEQ ID NO: 1225 - protein, 17 aa, synthetic construct
SVFNAGYKFGADLARRH

SEQ ID NO: 1226 - protein, 29 aa, synthetic construct
GGDWWSEILNFNRSQKYKHTSVGSNNIIV

SEQ ID NO: 1227 - protein, 29 aa, synthetic construct
GGDWWSEILNFNRSQKYKHTSVGSNNIIV

SEQ ID NO: 1228 - protein, 29 aa, synthetic construct
GGDWWSEILNFNRSQKYKHTSVGSNNIIV

SEQ ID NO: 1229 - protein, 29 aa, synthetic construct
GGDWWSEILNFNRSQKYKHTSVGSNNIIV

SEQ ID NO: 1230 - protein, 29 aa, synthetic construct
GGDWWSEILNFNRSQKYKHTSVGSNNIIV

SEQ ID NO: 1231 - protein, 31 aa, synthetic construct
AVIGGAVGAVAGAVGGVFVKDDPKVGIHVMY

SEQ ID NO: 1232 - protein, 23 aa, synthetic construct
ANHANGLFRRVLVGRMTGAGQVG

SEQ ID NO: 1233 - protein, 32 aa, synthetic construct; identical to SEQ ID NO: 925

SEQ ID NO: 1234 - protein, 30 aa, synthetic construct
TGGGWWKDLLHRFNVIEQNNTKGFNQPIQL

SEQ ID NO: 1235 - protein, 37 aa, synthetic construct
VSGGIGPLAAIALVKLGGAAVAGVGFAIAYGASKLRK

SEQ ID NO: 1236 - protein, 17 aa, synthetic construct
GYIGGGNHLCKGSAARF

SEQ ID NO: 1237 - protein, 17 aa, synthetic construct
GYIGGGNHLCKGSAARF

SEQ ID NO: 1238 - protein, 17 aa, synthetic construct
GYIGGGNHLCKGSAARF

SEQ ID NO: 1239 - protein, 17 aa, synthetic construct
GYIGGGNHLCKGSAARF

SEQ ID NO: 1240 - protein, 42 aa, synthetic construct
ATIINKLEQYAQTCTDPELKQMLQKDAQDARNTKQQLMTFLG

SEQ ID NO: 1241 - protein, 42 aa, synthetic construct; identical to SEQ ID NO: 1240

SEQ ID NO: 1242 - protein, 42 aa, synthetic construct
ATIINKLEQYAQVCTDPQLKQMLEKDAQDARNTKQQLMTFLG

SEQ ID NO: 1243 - protein, 42 aa, synthetic construct; identical to SEQ ID NO: 1240

SEQ ID NO: 1244 - protein, 42 aa, synthetic construct; identical to SEQ ID NO: 1240

SEQ ID NO: 1245 - protein, 42 aa, synthetic construct; identical to SEQ ID NO: 1240

SEQ ID NO: 1246 - protein, 42 aa, synthetic construct; identical to SEQ ID NO: 1240

SEQ ID NO: 1247 - protein, 42 aa, synthetic construct
ATIINKLEQYAQACTDPQLKQMLEKDAQDARNTKQQLMTFLG

SEQ ID NO: 1248 - protein, 18 aa, synthetic construct
SGYIGGGNHLCKGSAARF

SEQ ID NO: 1249 - protein, 42 aa, synthetic construct; identical to SEQ ID NO: 1240

SEQ ID NO: 1250 - protein, 42 aa, synthetic construct; identical to SEQ ID NO: 1247

SEQ ID NO: 1251 - protein, 42 aa, synthetic construct; identical to SEQ ID NO: 1240

SEQ ID NO: 1252 - protein, 12 aa, synthetic construct
TGFYNGYRNAGR

SEQ ID NO: 1253 - protein, 42 aa, synthetic construct; identical to SEQ ID NO: 1240

SEQ ID NO: 1254 - protein, 42 aa, synthetic construct; identical to SEQ ID NO: 1240

SEQ ID NO: 1255 - protein, 42 aa, synthetic construct; identical to SEQ ID NO: 1240

SEQ ID NO: 1256 - protein, 42 aa, synthetic construct; identical to SEQ ID NO: 1247

SEQ ID NO: 1257 - protein, 42 aa, synthetic construct; identical to SEQ ID NO: 1247

SEQ ID NO: 1258 - protein, 42 aa, synthetic construct; identical to SEQ ID NO: 1240

SEQ ID NO: 1259 - protein, 42 aa, synthetic construct; identical to SEQ ID NO: 1240

SEQ ID NO: 1260 - protein, 42 aa, synthetic construct; identical to SEQ ID NO: 1240

SEQ ID NO: 1261 - protein, 42 aa, synthetic construct; identical to SEQ ID NO: 1240

SEQ ID NO: 1262 - protein, 42 aa, synthetic construct; identical to SEQ ID NO: 1247

SEQ ID NO: 1263 - protein, 42 aa, synthetic construct; identical to SEQ ID NO: 1240

SEQ ID NO: 1264 - protein, 42 aa, synthetic construct; identical to SEQ ID NO: 1240

SEQ ID NO: 1265 - protein, 42 aa, synthetic construct; identical to SEQ ID NO: 1240

SEQ ID NO: 1266 - protein, 17 aa, synthetic construct
GYIGGGNHLCKGSAARF

SEQ ID NO: 1267 - protein, 17 aa, synthetic construct
GYIGGGNHLCKGSAARF

SEQ ID NO: 1268 - protein, 17 aa, synthetic construct
GYIGGGNHLCKGSAARF

SEQ ID NO: 1269 - protein, 17 aa, synthetic construct
GYIGGGNHLCKGSAARF

SEQ ID NO: 1270 - protein, 17 aa, synthetic construct
GYIGGGNHLCKGSAARF

SEQ ID NO: 1271 - protein, 17 aa, synthetic construct
GYIGGGNHLCKGSAARF

SEQ ID NO: 1272 - protein, 17 aa, synthetic construct
GYIGGGNHLCKGSAARF

SEQ ID NO: 1273 - protein, 17 aa, synthetic construct
GYIGGGNHLCKGSAARF

SEQ ID NO: 1274 - protein, 27 aa, synthetic construct
AAGWKACSSIYGMPFVDCQRSSGPCSR

SEQ ID NO: 1275 - protein, 15 aa, synthetic construct
GGLGGAVAYGATCWW

SEQ ID NO: 1276 - protein, 17 aa, synthetic construct
GYIGGGNHLCKGSAARF

SEQ ID NO: 1277 - protein, 17 aa, synthetic construct
GYIGGGNHLCKGSAARF

SEQ ID NO: 1278 - protein, 17 aa, synthetic construct
GYIGGGNHLCKGSPARF

SEQ ID NO: 1279 - protein, 17 aa, synthetic construct
GYIGGGNHLCKGSAARF

SEQ ID NO: 1280 - protein, 21 aa, synthetic construct
KLFQDAADSAMKNKQDLMRFL

SEQ ID NO: 1281 - protein, 42 aa, synthetic construct
ATIINKLDQYAQACTDPELKQMLQKDAQDARNTKQQLMTFLG

SEQ ID NO: 1282 - protein, 42 aa, synthetic construct; identical to SEQ ID NO: 1281

SEQ ID NO: 1283 - protein, 42 aa, synthetic construct; identical to SEQ ID NO: 1281

SEQ ID NO: 1284 - protein, 42 aa, synthetic construct; identical to SEQ ID NO: 1281

SEQ ID NO: 1285 - protein, 42 aa, synthetic construct; identical to SEQ ID NO: 1281

SEQ ID NO: 1286 - protein, 42 aa, synthetic construct; identical to SEQ ID NO: 1281

SEQ ID NO: 1287 - protein, 42 aa, synthetic construct; identical to SEQ ID NO: 1281

SEQ ID NO: 1288 - protein, 42 aa, synthetic construct; identical to SEQ ID NO: 1281

SEQ ID NO: 1289 - protein, 42 aa, synthetic construct; identical to SEQ ID NO: 1281

SEQ ID NO: 1290 - protein, 36 aa, synthetic construct
MQDYAQKAEDMEVKQFFQKSAQSAQQTKQQLMQFLQ

SEQ ID NO: 1291 - protein, 42 aa, synthetic construct
IIGGLGGLASGLKFCKLPHLVLTGECVVGFTVGGAYLGYTAN

SEQ ID NO: 1292 - protein, 36 aa, synthetic construct
MDEYARQATDAQVKQFFQKSAQSAADCKNQLMQYLQ

SEQ ID NO: 1293 - protein, 21 aa, synthetic construct
SITNFWKKYFNHDSSTVNRRH

SEQ ID NO: 1294 - protein, 21 aa, synthetic construct
SITNFWKKYFNHDSSTVNRRH

SEQ ID NO: 1295 - protein, 20 aa, synthetic construct
AVASLLGGGGHINITIHRQN

SEQ ID NO: 1296 - protein, 25 aa, synthetic construct
AEIKKLFQDAADSAMKNKQDLMKFL

SEQ ID NO: 1297 - protein, 21 aa, synthetic construct
KLFQDAADSAMKNKQELMRFL

SEQ ID NO: 1298 - protein, 21 aa, synthetic construct
KLFQDAADSAMKNKQELMRFL

SEQ ID NO: 1299 - protein, 21 aa, synthetic construct
KLFQDAADSAMKNKQELMRFL

SEQ ID NO: 1300 - protein, 25 aa, synthetic construct
AEIKKLFQDAADSAMKNKQDLMKFL

SEQ ID NO: 1301 - protein, 21 aa, synthetic construct
KLFQDAADSAMKNKQELMRFL

SEQ ID NO: 1302 - protein, 13 aa, synthetic construct
VINEFVKGFHHAW

SEQ ID NO: 1303 - protein, 13 aa, synthetic construct
VINEFVKGFHHAW

SEQ ID NO: 1304 - protein, 26 aa, synthetic construct
GWLEDLLKHFSGYNSLTKGDSNNTLG

SEQ ID NO: 1305 - protein, 42 aa, synthetic construct
AGAAAGAAVMGGTAIALGAAAGPVGFAAGVGMAILTRPGHAR

SEQ ID NO: 1306 - protein, 26 aa, synthetic construct
GWLEDLLKHFSGYNSLTKGDSNNTLG

SEQ ID NO: 1307 - protein, 33 aa, synthetic construct
AGFVEWLRQLFTPAVPDEPDRWVHEELNHNAQY

SEQ ID NO: 1308 - protein, 34 aa, synthetic construct; identical to SEQ ID NO: 1173

SEQ ID NO: 1309 - protein, 24 aa, synthetic construct
WAGAGAAGFSGGIAVGLNRVNRNN

SEQ ID NO: 1310 - protein, 33 aa, synthetic construct
GQTAQQVVNQLNGVTGFHAYNHNPYGSGGTPND

SEQ ID NO: 1311 - protein, 33 aa, synthetic construct; identical to SEQ ID NO: 1310

SEQ ID NO: 1312 - protein, 20 aa, synthetic construct
KTFKEIVSRIEQRIRQNESL

SEQ ID NO: 1313 - protein, 19 aa, synthetic construct
HLLAGLWDWFKRHDPYKQA

SEQ ID NO: 1314 - protein, 19 aa, synthetic construct
HLLAGLWDWFKRHDPYKQA

SEQ ID NO: 1315 - protein, 19 aa, synthetic construct
HLLAGLWDWFKRHDPYKQA

SEQ ID NO: 1316 - protein, 21 aa, synthetic construct
KLFQDAAQSAMKNKQDLMKFL

SEQ ID NO: 1317 - protein, 21 aa, synthetic construct
KLFQDAAQSAMKNKQDLMKFL

SEQ ID NO: 1318 - protein, 22 aa, synthetic construct
SLATEWSTILTHATRRGSGIGY

SEQ ID NO: 1319 - protein, 17 aa, synthetic construct
FDLYRGGAHQGSYTLCK

SEQ ID NO: 1320 - protein, 29 aa, synthetic construct
TLVKELYEFAKELYDFGRGFIKGWQDATR

SEQ ID NO: 1321 - protein, 42 aa, synthetic construct
EELENIAGGFGHPTMDDPRYKEQMRINTMLKRGMKMDEIQKR

SEQ ID NO: 1322 - protein, 40 aa, synthetic construct
QKGMSQVRGGVQEESLKDDKKLEPDWGNTNYCESTRPVRR

SEQ ID NO: 1323 - protein, 13 aa, synthetic construct
SGPAWLSRYLHKR

SEQ ID NO: 1324 - protein, 22 aa, synthetic construct
QTGPEYGDTFLDGDNDRLQDES

SEQ ID NO: 1325 - protein, 28 aa, synthetic construct
WKIATAAVGSIGLVFAAGVGLGYLANRP

SEQ ID NO: 1326 - protein, 12 aa, synthetic construct
EVGKSAAERARR

SEQ ID NO: 1327 - protein, 34 aa, synthetic construct
TLAGELAQLGAKALQSLWELGKMVSDTQSSIAKK

SEQ ID NO: 1328 - protein, 31 aa, synthetic construct
GCWDGYYEAAQKDNSCGSFGGGTSRGYGASR

SEQ ID NO: 1329 - protein, 31 aa, synthetic construct; identical to SEQ ID NO: 1328

SEQ ID NO: 1330 - protein, 22 aa, synthetic construct
KYVKNYLDFIKKAIDIFRPMPI

SEQ ID NO: 1331 - protein, 28 aa, synthetic construct
VEKAVQRGVDASRLRDQVEAYMEDERKK

SEQ ID NO: 1332 - protein, 28 aa, synthetic construct
VEKAVQRGVDASRLRDQVEAYMEDERKK

SEQ ID NO: 1333 - protein, 21 aa, synthetic construct
KLFQDAADSAMKNRQDLMKFL

SEQ ID NO: 1334 - protein, 21 aa, synthetic construct
KLFQDAADSAMKNRQDLMKFL

SEQ ID NO: 1335 - protein, 39 aa, synthetic construct
NYIARVFSAGSSIINNVVTIVDTVKKINSFVPDMGSTKK

SEQ ID NO: 1336 - protein, 42 aa, synthetic construct
GNVAKKLEAYAASCTDPELKAILERDAQAAKQAQQDLISYLN

SEQ ID NO: 1337 - protein, 25 aa, synthetic construct
LAALGGAVFAFGYEVGKDMAARDAR

SEQ ID NO: 1338 - protein, 30 aa, synthetic construct
TGGGWWEELLHETILSKFKITKALELPIQL

SEQ ID NO: 1339 - protein, 30 aa, synthetic construct; identical to SEQ ID NO: 1338

SEQ ID NO: 1340 - protein, 30 aa, synthetic construct; identical to SEQ ID NO: 1338

SEQ ID NO: 1341 - protein, 30 aa, synthetic construct; identical to SEQ ID NO: 1338

SEQ ID NO: 1342 - protein, 30 aa, synthetic construct; identical to SEQ ID NO: 1338

SEQ ID NO: 1343 - protein, 30 aa, synthetic construct; identical to SEQ ID NO: 1338

SEQ ID NO: 1344 - protein, 30 aa, synthetic construct; identical to SEQ ID NO: 1338

SEQ ID NO: 1345 - protein, 28 aa, synthetic construct
KVAEAIIKAITHPPTGPGPGPTVPRRMS

SEQ ID NO: 1346 - protein, 24 aa, synthetic construct
GWAGRIAGKVAGGVRSLADGIANG

SEQ ID NO: 1347 - protein, 41 aa, synthetic construct
GGEPVSDGYEEVYYDQGGVTHVYHCRNGNCVYIRSMEGMIA

SEQ ID NO: 1348 - protein, 21 aa, synthetic construct
KLFQDAADSAMKNKQELMKFL

SEQ ID NO: 1349 - protein, 21 aa, synthetic construct
KLFQDAADSAMKNKQELMKFL

SEQ ID NO: 1350 - protein, 17 aa, synthetic construct
GYIGGGNHLCKGSAARF

SEQ ID NO: 1351 - protein, 32 aa, synthetic construct
RNGARIAGRLVGIALMGPLGGIYYCGKDMGWW

SEQ ID NO: 1352 - protein, 32 aa, synthetic construct; identical to SEQ ID NO: 1351

SEQ ID NO: 1353 - protein, 30 aa, synthetic construct
GVIGRLIKAAANATIAMAEDLIDRWRGPNA

SEQ ID NO: 1354 - protein, 25 aa, synthetic construct
KYLSSVIAEMTGLEPAVGIITLTYQ

SEQ ID NO: 1355 - protein, 25 aa, synthetic construct
KYLSSVIAEMTGLEPAVGIITLTYQ

SEQ ID NO: 1356 - protein, 21 aa, synthetic construct
KLFQDAADSAMKNKQTLMKFL

SEQ ID NO: 1357 - protein, 27 aa, synthetic construct
GRELGHKIGKAFYGACAMYGVYVLFCL

SEQ ID NO: 1358 - protein, 30 aa, synthetic construct
TGGGWWEDFLHEISLSKFKITKALELPIQL

SEQ ID NO: 1359 - protein, 25 aa, synthetic construct
NAVNAMVAFADGVKEGVQAIKDLKS

SEQ ID NO: 1360 - protein, 17 aa, synthetic construct
GYIGGGNHLCKGSAARF

SEQ ID NO: 1361 - protein, 21 aa, synthetic construct
KLFQDAADSAMKNKQELMKFL

SEQ ID NO: 1362 - protein, 22 aa, synthetic construct
KLFQDAADSAMKNKQDLMKFLS

SEQ ID NO: 1363 - protein, 22 aa, synthetic construct
KLFQDAADSAMKNKQDLMKFLS

SEQ ID NO: 1364 - protein, 13 aa, synthetic construct
VINEFVKGFRQAW

SEQ ID NO: 1365 - protein, 13 aa, synthetic construct
VINEFVKGFRQAW

SEQ ID NO: 1366 - protein, 22 aa, synthetic construct
AAIGGLYKAGEAIGKGLYHLFN

SEQ ID NO: 1367 - protein, 18 aa, synthetic construct
SAFGAGYKFGSDLARRGR

SEQ ID NO: 1368 - protein, 29 aa, synthetic construct
GAAAVVTTGGAALFAASFILGSIGTARSC

SEQ ID NO: 1369 - protein, 29 aa, synthetic construct
ALTGLAAGVAAGLNPLVGGLATAACFLLN

SEQ ID NO: 1370 - protein, 29 aa, synthetic construct
IADKFVDAFGDYSTNSCRKNHRQWFCIQV

SEQ ID NO: 1371 - protein, 36 aa, synthetic construct
GLGAIARSWAQGGFVAPAMSLSIDVLNELKRKENLP

SEQ ID NO: 1372 - protein, 36 aa, synthetic construct
GLGAIARSWAQGGFATPAMSLGIDVLNELKRKENLP

SEQ ID NO: 1373 - protein, 38 aa, synthetic construct
AGGRAVSAIVDGRAPGTVDDWGINIAIFEGRISGPYIV

SEQ ID NO: 1374 - protein, 42 aa, synthetic construct
RAIVNLIGNGIATVVVAKSENEFDDEKYKRVVEEMKREKIAG

SEQ ID NO: 1375 - protein, 42 aa, synthetic construct; identical to SEQ ID NO: 1374

SEQ ID NO: 1376 - protein, 42 aa, synthetic construct; identical to SEQ ID NO: 1374

SEQ ID NO: 1377 - protein, 42 aa, synthetic construct; identical to SEQ ID NO: 1374

SEQ ID NO: 1378 - protein, 42 aa, synthetic construct
LSGAAGAVEAAMSCAPAGPYVIAACAVGGAIYGGFWGYVFGS

SEQ ID NO: 1379 - protein, 42 aa, synthetic construct; identical to SEQ ID NO: 1378

SEQ ID NO: 1380 - protein, 27 aa, synthetic construct
GGGNGVIKTISHECHMNTWQFIFTCCS

SEQ ID NO: 1381 - protein, 40 aa, synthetic construct
IANKLEAYAQNCTDPQLVSILQQDAQDAKAAQQKLMTFLG

SEQ ID NO: 1382 - protein, 25 aa, synthetic construct
GIVGTLITFFAGVVDGYLRPLKCEE

SEQ ID NO: 1383 - protein, 28 aa, synthetic construct
GFWDGYYEVAQRDSSFGGGSSRGHGATR

SEQ ID NO: 1384 - protein, 18 aa, synthetic construct
DAFNSFMKGFNQGYNKKS

SEQ ID NO: 1385 - protein, 36 aa, synthetic construct; identical to SEQ ID NO: 1371

SEQ ID NO: 1386 - protein, 22 aa, synthetic construct
SLATEWSTILTHATRRAYGIGY

SEQ ID NO: 1387 - protein, 24 aa, synthetic construct
CAKLAGAGITAGIAVAGIYYSQKK

SEQ ID NO: 1388 - protein, 27 aa, synthetic construct
VGAGFKAALDGVFVYQWYKKLKENGWI

SEQ ID NO: 1389 - protein, 25 aa, synthetic construct
GVLNAAVQIFNAGYKFGSDFARRGR

SEQ ID NO: 1390 - protein, 25 aa, synthetic construct
GVLNAAVQIFNAGYKFGSDFARRGR

SEQ ID NO: 1391 - protein, 25 aa, synthetic construct
GVLNAAVQIFNAGYKFGSDFARRGR

SEQ ID NO: 1392 - protein, 25 aa, synthetic construct
GVLNAAVQIFNAGYKFGSDLARRGR

SEQ ID NO: 1393 - protein, 25 aa, synthetic construct
GVLNAAVQIFNAGYKFGSDLARRGR

SEQ ID NO: 1394 - protein, 25 aa, synthetic construct
GVLNAAVQIFNAGYKFGSDFARRGR

SEQ ID NO: 1395 - protein, 25 aa, synthetic construct
GVLNAAVQIFSAGYKFGSDLARRGR

SEQ ID NO: 1396 - protein, 25 aa, synthetic construct
GVLNAAVQIFNAGYKFGSDFARRGR

SEQ ID NO: 1397 - protein, 25 aa, synthetic construct
GVLNAAVQIFNAGYKFGSDFARRGR

SEQ ID NO: 1398 - protein, 37 aa, synthetic construct
KVFNAIARIFSGASSIINNVNVIYQSFLTLGDLLKSF

SEQ ID NO: 1399 - protein, 25 aa, synthetic construct
GVLNAAVQIFNAGYKFGSDLARRGR

SEQ ID NO: 1400 - protein, 25 aa, synthetic construct
GVLNAAVQIFNAGYKFGSDFARRGR

SEQ ID NO: 1401 - protein, 16 aa, synthetic construct
NVLNGMLGAIGRPPIT

SEQ ID NO: 1402 - protein, 17 aa, synthetic construct
SVFNAGYKFGADLARRH

SEQ ID NO: 1403 - protein, 15 aa, synthetic construct
GLVDGFIGIFRGLGH

SEQ ID NO: 1404 - protein, 15 aa, synthetic construct
AAGAAVGIAAGAAAN

SEQ ID NO: 1405 - protein, 36 aa, synthetic construct
WGVFKSVIGTFGPSAPHPEACIPKSSWNPVPPVPCP

SEQ ID NO: 1406 - protein, 34 aa, synthetic construct
EDVATVIQSLDDQIQQMTINLNETSAKLRSQLGI

SEQ ID NO: 1407 - protein, 16 aa, synthetic construct
SGAKTGWEIANKYKIY

SEQ ID NO: 1408 - protein, 39 aa, synthetic construct
EGGSQYCEHLQQMTRGEEAQGWSNEKWDEWAEAWDTHCW

SEQ ID NO: 1409 - protein, 14 aa, synthetic construct
GFWGGAATGAATFC

SEQ ID NO: 1410 - protein, 39 aa, synthetic construct
GVIGAVATTVGGVVNTVGTVVGNTVKFGLTQLFTILGSL

SEQ ID NO: 1411 - protein, 41 aa, synthetic construct
GALGNALNGLGTWANMMNGGGFVNQWQVYANKGKINQYRPY

SEQ ID NO: 1412 - protein, 36 aa, synthetic construct
NYIARIFSGASSIINNVNVIYQSFVSLNSLLGSFGK

SEQ ID NO: 1413 - protein, 36 aa, synthetic construct; identical to SEQ ID NO: 1412

SEQ ID NO: 1414 - protein, 29 aa, synthetic construct
AVATGGIGLYAAAVGFAVAPASWGYSCFT

SEQ ID NO: 1415 - protein, 36 aa, synthetic construct
AGGAIFGGIAGLGVGALLGGYGAGVMITICCDEMNN

SEQ ID NO: 1416 - protein, 22 aa, synthetic construct
GGGWIDGIKKIINLDKLNFLKL

SEQ ID NO: 1417 - protein, 17 aa, synthetic construct
GVTAVAAGLGIGYLVNR

SEQ ID NO: 1418 - protein, 21 aa, synthetic construct
GIFEDAVKWCDKLFSTGRHFA

SEQ ID NO: 1419 - protein, 12 aa, synthetic construct
GEGMDPVAGGFA

SEQ ID NO: 1420 - protein, 13 aa, synthetic construct
NGPAWLNRYLTKR

SEQ ID NO: 1421 - protein, 29 aa, synthetic construct
LVSGGIALGSRCGYPLFADGEFYVTHRDC

SEQ ID NO: 1422 - protein, 12 aa, synthetic construct
GKDGIAGGKSVL

SEQ ID NO: 1423 - protein, 38 aa, synthetic construct
AGYGAAFGTAFGPGVGTAVGAAVGLGLGAVGIIGCGGN

SEQ ID NO: 1424 - protein, 17 aa, synthetic construct
NWGTQGHAGWHTGGVAP

SEQ ID NO: 1425 - protein, 35 aa, synthetic construct
QFVAGGIGAAAGGVAGGAIYDYGGTAEFGKNRTLS

SEQ ID NO: 1426 - protein, 35 aa, synthetic construct; identical to SEQ ID NO: 1425

SEQ ID NO: 1427 - protein, 16 aa, synthetic construct
GGATSGWSLADKSHSL

SEQ ID NO: 1428 - protein, 16 aa, synthetic construct
GGATSGWSLADKSHSL

SEQ ID NO: 1429 - protein, 16 aa, synthetic construct
GGATSGWSLADKSHSL

SEQ ID NO: 1430 - protein, 16 aa, synthetic construct
GGATSGWSLADKSHSL

SEQ ID NO: 1431 - protein, 16 aa, synthetic construct
GGATSGWSLADKSHSL

SEQ ID NO: 1432 - protein, 16 aa, synthetic construct
GGATSGWSLADKSHSL

SEQ ID NO: 1433 - protein, 16 aa, synthetic construct
GGATSGWSLADKSHSL

SEQ ID NO: 1434 - protein, 16 aa, synthetic construct
GGATSGWSLADKSHSL

SEQ ID NO: 1435 - protein, 16 aa, synthetic construct
GGATSGWSLADKSHSL

SEQ ID NO: 1436 - protein, 16 aa, synthetic construct
GGATSGWSLADKSHSL

SEQ ID NO: 1437 - protein, 16 aa, synthetic construct
GGATSGWSLADKSHSL

SEQ ID NO: 1438 - protein, 16 aa, synthetic construct
GGATSGWSLADKSHSL

SEQ ID NO: 1439 - protein, 29 aa, synthetic construct
AVFGSAAGPVGSLVGAIIGGGVYWLIDQF

SEQ ID NO: 1440 - protein, 16 aa, synthetic construct
GGATSGWSLADKSHSL

SEQ ID NO: 1441 - protein, 16 aa, synthetic construct
GGATSGWSLADKSHSL

SEQ ID NO: 1442 - protein, 18 aa, synthetic construct
GGDVVKTLTRLFNQKYLW

SEQ ID NO: 1443 - protein, 30 aa, synthetic construct
DALSEAGKNIRDDWREIRDDWREIREILKD

SEQ ID NO: 1444 - protein, 24 aa, synthetic construct
NILHSIGNAFSSAYHGLVDGWNNH

SEQ ID NO: 1445 - protein, 26 aa, synthetic construct
DYINGFLKGFGGQRTLPTKDYNIPQA

SEQ ID NO: 1446 - protein, 26 aa, synthetic construct
DYINGFLKGFGGQRTLPTKDYNIPQA

SEQ ID NO: 1447 - protein, 26 aa, synthetic construct
DYINGFLKGFGGQRTLPTKDYNIPQA

SEQ ID NO: 1448 - protein, 26 aa, synthetic construct
DYINGFLKGFGGQRTLPTKDYNIPQA

SEQ ID NO: 1449 - protein, 26 aa, synthetic construct
DYINGFLKGFGGQRTLPTKDYNIPQV

SEQ ID NO: 1450 - protein, 26 aa, synthetic construct
DYINGFLKGFGGQRTLPTKDYNIPQV

SEQ ID NO: 1451 - protein, 19 aa, synthetic construct
VLAGGGGFAAGYGLSRWLG

SEQ ID NO: 1452 - protein, 18 aa, synthetic construct
IDSFKKGADKANREWDNK

SEQ ID NO: 1453 - protein, 14 aa, synthetic construct
TDAGEVGTRGEATA

SEQ ID NO: 1454 - protein, 42 aa, synthetic construct
RAIVNLIGDGIATVVVAKSENEFDDEKYKRVVEEMKREKIAG

SEQ ID NO: 1455 - protein, 32 aa, synthetic construct
LEDLEQVAGGYSLREKKKVETTAISDDTISRI

SEQ ID NO: 1456 - protein, 25 aa, synthetic construct
QIKSFFEDAVTSSMDNKQTLMKFLQ

SEQ ID NO: 1457 - protein, 23 aa, synthetic construct
YKKVAKKIEDYVDNGPLISVGMM

SEQ ID NO: 1458 - protein, 32 aa, synthetic construct
WSTMSRACGGVGDDEWSTMSDSCKGGEKVIMR

SEQ ID NO: 1459 - protein, 29 aa, synthetic construct
ELLGGAGHGVNTISAECRWNSLQAIFSCC

SEQ ID NO: 1460 - protein, 29 aa, synthetic construct
ELLGGAGHGVNTISAECRWNSLQAIFSCC

SEQ ID NO: 1461 - protein, 29 aa, synthetic construct
ELLGGAGHGVNTISAECRWNSLQAIFSCC

SEQ ID NO: 1462 - protein, 29 aa, synthetic construct
ELLGGAGHGVNTISAECRWNSLQAIFSCC

SEQ ID NO: 1463 - protein, 29 aa, synthetic construct
ELLGGAGHGVNTISAECRWNSLQAIFSCC

SEQ ID NO: 1464 - protein, 20 aa, synthetic construct
MGQGALNSYRDAWKYGFRAG

SEQ ID NO: 1465 - protein, 18 aa, synthetic construct
EIYDSIVDFVKRGIKALL

SEQ ID NO: 1466 - protein, 15 aa, synthetic construct
SNFSDAYSSFYNATH

SEQ ID NO: 1467 - protein, 35 aa, synthetic construct
SPFEEVISHAAKHGSLAELLDMVQTEIAKMAKEAA

SEQ ID NO: 1468 - protein, 23 aa, synthetic construct
ALISAGVTTLAGAAALGFYNGSK

SEQ ID NO: 1469 - protein, 23 aa, synthetic construct
ALISAGVTTLAGAAALGFYNGSK

SEQ ID NO: 1470 - protein, 23 aa, synthetic construct
ALISAGVTTLAGAAALGFYNGSK

SEQ ID NO: 1471 - protein, 23 aa, synthetic construct
ALISAGVTTLAGAAALGFYNGSK

SEQ ID NO: 1472 - protein, 36 aa, synthetic construct
LDGVVGGLATYYEMNGNWYIVGHDKSGKATGVVRVT

SEQ ID NO: 1473 - protein, 34 aa, synthetic construct
HKVADTYNAIAKTTQESGFMASPVGAGSKGAGGK

SEQ ID NO: 1474 - protein, 32 aa, synthetic construct
GGGYGLWAALGGVITFLISAIEGFLNPIECGK

SEQ ID NO: 1475 - protein, 39 aa, synthetic construct; identical to SEQ ID NO: 1408

SEQ ID NO: 1476 - protein, 38 aa, synthetic construct
YNKMEKILEKLSPEELDSIKGGTWVLTDKGWVWIEGAI

SEQ ID NO: 1477 - protein, 38 aa, synthetic construct; identical to SEQ ID NO: 1476

SEQ ID NO: 1478 - protein, 38 aa, synthetic construct; identical to SEQ ID NO: 1476

SEQ ID NO: 1479 - protein, 38 aa, synthetic construct; identical to SEQ ID NO: 1476

SEQ ID NO: 1480 - protein, 38 aa, synthetic construct; identical to SEQ ID NO: 1476

SEQ ID NO: 1481 - protein, 38 aa, synthetic construct; identical to SEQ ID NO: 1476

SEQ ID NO: 1482 - protein, 40 aa, synthetic construct
DQLGDVVGGGTTNTCNNVPAQELEAIPTIGSCQACSCNGG

SEQ ID NO: 1483 - protein, 39 aa, synthetic construct
DQLGDVVGGGTTNTCNGTPPAQELEAIPTINSCQACSCN

SEQ ID NO: 1484 - protein, 38 aa, synthetic construct
ELLRAVVGGVADDEVIKLSQELNVYIVEVQRKLVERES

SEQ ID NO: 1485 - protein, 28 aa, synthetic construct
AEAARAISDCQQEGFEGPAWLTRYLNKR

SEQ ID NO: 1486 - protein, 42 aa, synthetic construct
LAGAAGAVEAAMSCAPAGPYVIAACAVGGAIYGGFWGYVFGS

SEQ ID NO: 1487 - protein, 42 aa, synthetic construct; identical to SEQ ID NO: 1486

SEQ ID NO: 1488 - protein, 27 aa, synthetic construct
GRELGHKIGKAFYGACAMYGVYVLFCL

SEQ ID NO: 1489 - protein, 30 aa, synthetic construct
TGGGWWEDLLHEIIFSKFKITKALELPIQL

SEQ ID NO: 1490 - protein, 23 aa, synthetic construct
QGGENWAKTLADWYIGFRRGLGF

SEQ ID NO: 1491 - protein, 23 aa, synthetic construct
QGGESWVKTLADWYIGFRRGFGF

SEQ ID NO: 1492 - protein, 23 aa, synthetic construct
QGGESWVKTLADWYIGFRRGFGF

SEQ ID NO: 1493 - protein, 23 aa, synthetic construct
QGGESWVKTLADWYIGFRRGFGF

SEQ ID NO: 1494 - protein, 23 aa, synthetic construct
LATDAFNHADQLIDGFKQGWQRP

SEQ ID NO: 1495 - protein, 27 aa, synthetic construct
GRELGHKIGKAFYGACAMYGVYVLFCL

SEQ ID NO: 1496 - protein, 40 aa, synthetic construct
TAAGSIITGVGAAVVFGSGPVGAAVGLAGALILSNPSKVS

SEQ ID NO: 1497 - protein, 40 aa, synthetic construct; identical to SEQ ID NO: 1496

SEQ ID NO: 1498 - protein, 23 aa, synthetic construct
QGGENWVKTLADWYIGFRRGFGF

SEQ ID NO: 1499 - protein, 31 aa, synthetic construct
TFWEKLIEYIQSIPGYDPMVEYQSRTYEVYM

SEQ ID NO: 1500 - protein, 23 aa, synthetic construct
QGGESWAKTLADWYLGFRRGLGF

SEQ ID NO: 1501 - protein, 27 aa, synthetic construct
GRELGHKIGKAFYGACAMYGVYVLFCL

SEQ ID NO: 1502 - protein, 27 aa, synthetic construct
GRELGHKIGKAFYGACAMYGVYVLFCL

SEQ ID NO: 1503 - protein, 18 aa, synthetic construct
TTLDAAAHQALEGWVSIP

SEQ ID NO: 1504 - protein, 22 aa, synthetic construct
SLATEWSTILTHATRRAYGIGY

SEQ ID NO: 1505 - protein, 29 aa, synthetic construct
TLIKDLYEFAKELWDFGRGFADGWRDATR

SEQ ID NO: 1506 - protein, 19 aa, synthetic construct
SGVEGILDKVKARAQAYMK

SEQ ID NO: 1507 - protein, 27 aa, synthetic construct
ATLTGGGGGIPNPIRVQPDGDVSVDFG

SEQ ID NO: 1508 - protein, 27 aa, synthetic construct
ATLTGGGGGIPNPIRVQPDGDVSVDFG

SEQ ID NO: 1509 - protein, 27 aa, synthetic construct
ATLTGGGGGIPNPIRVQPDGDVSVDFG

SEQ ID NO: 1510 - protein, 27 aa, synthetic construct
ATLTGGGGGIPNPIRVQPDGDVSVDFG

SEQ ID NO: 1511 - protein, 13 aa, synthetic construct
SGPAWLNRYLNKR

SEQ ID NO: 1512 - protein, 42 aa, synthetic construct
GGLAVWEDGYSRWLYYREFAPYMRQGALNSYIDAWKYGFRAG

SEQ ID NO: 1513 - protein, 42 aa, synthetic construct; identical to SEQ ID NO: 1512

SEQ ID NO: 1514 - protein, 42 aa, synthetic construct; identical to SEQ ID NO: 1512

SEQ ID NO: 1515 - protein, 42 aa, synthetic construct; identical to SEQ ID NO: 1512

SEQ ID NO: 1516 - protein, 23 aa, synthetic construct
AFGAAVGAVVGSDAATKFLKLFA

SEQ ID NO: 1517 - protein, 17 aa, synthetic construct
HIVDAVVSFGEGFLNAI

SEQ ID NO: 1518 - protein, 42 aa, synthetic construct; identical to SEQ ID NO: 1512

SEQ ID NO: 1519 - protein, 42 aa, synthetic construct; identical to SEQ ID NO: 1512

SEQ ID NO: 1520 - protein, 42 aa, synthetic construct; identical to SEQ ID NO: 1512

SEQ ID NO: 1521 - protein, 42 aa, synthetic construct; identical to SEQ ID NO: 1512

SEQ ID NO: 1522 - protein, 34 aa, synthetic construct
EGGNQMAEDMGSMMGEAVGRAIKMFLFCAAIYAL

SEQ ID NO: 1523 - protein, 13 aa, synthetic construct
SGPAWLNRYLNKR

SEQ ID NO: 1524 - protein, 13 aa, synthetic construct
SGPAWLNRYLNKR

SEQ ID NO: 1525 - protein, 16 aa, synthetic construct
SGAKTGWEIANKYKIY

SEQ ID NO: 1526 - protein, 41 aa, synthetic construct
GICATAGAIIGGAFGAVPGALGGAVLGAQYCGGTWVIIRTH

SEQ ID NO: 1527 - protein, 18 aa, synthetic construct
GAVAAGVGLVAAAKELFS

SEQ ID NO: 1528 - protein, 16 aa, synthetic construct
SGAKTGWEIANKYKIY

SEQ ID NO: 1529 - protein, 16 aa, synthetic construct
SGAKTGWEIANKYKIY

SEQ ID NO: 1530 - protein, 16 aa, synthetic construct
SGAKTGWEIANKYKIY

SEQ ID NO: 1531 - protein, 16 aa, synthetic construct
SGAKTGWEIANKYKIY

SEQ ID NO: 1532 - protein, 16 aa, synthetic construct
SGAKTGWEIANKYKIY

SEQ ID NO: 1533 - protein, 16 aa, synthetic construct
SGAKTGWEIANKYKIY

SEQ ID NO: 1534 - protein, 27 aa, synthetic construct
GGGDGVFRTISHECHMNTWMFIFTCCS

SEQ ID NO: 1535 - protein, 28 aa, synthetic construct
AAIEGGFNPLKAIGDAAHDFAKGFHRGW

SEQ ID NO: 1536 - protein, 37 aa, synthetic construct
GGAASGFTYCASNGVWHPYILAGCAGVGAVGSVVFPH

SEQ ID NO: 1537 - protein, 35 aa, synthetic construct
ECVAGGVEFLSYRELVLKDIKKQQQRRVDILVSRM

SEQ ID NO: 1538 - protein, 41 aa, synthetic construct
GVLATAAGKIAYCIGYGALNRGCDISGNPYPWDGFISATVR

SEQ ID NO: 1539 - protein, 26 aa, synthetic construct
KVAGAVVTIGGALYGAGYAIGKALRP

SEQ ID NO: 1540 - protein, 22 aa, synthetic construct
IAGSAFAGLSAGVAIGISRNKR

SEQ ID NO: 1541 - protein, 13 aa, synthetic construct
LGAGLYQVGKNLK

SEQ ID NO: 1542 - protein, 22 aa, synthetic construct
IAGSAFAGLSAGVAIGISRNKR

SEQ ID NO: 1543 - protein, 13 aa, synthetic construct
LGAGLYQVGKNLK

SEQ ID NO: 1544 - protein, 13 aa, synthetic construct
LGAGLYQVGKNLK

SEQ ID NO: 1545 - protein, 36 aa, synthetic construct
NYIARVFSAGSSIVNNINVIVTTFQTLSTFSLTAKN

SEQ ID NO: 1546 - protein, 22 aa, synthetic construct
IAGSAFAGLSAGVAIGISRNKR

SEQ ID NO: 1547 - protein, 22 aa, synthetic construct
IAGSAFAGLSAGVAIGISRNKR

SEQ ID NO: 1548 - protein, 22 aa, synthetic construct
IAGSAFAGLSAGVAIGISRNKR

SEQ ID NO: 1549 - protein, 41 aa, synthetic construct
GICATAGAVIGGALGSVPGALGGAVLGAQYCGGTWVILRTH

SEQ ID NO: 1550 - protein, 23 aa, synthetic construct
QAINDFVDGFNETPCSCEEIKEN

SEQ ID NO: 1551 - protein, 18 aa, synthetic construct
RFGKGVVEGYSEAEAEDK

SEQ ID NO: 1552 - protein, 20 aa, synthetic construct
GGFRSYGRQLDGQRRNNTQS

SEQ ID NO: 1553 - protein, 27 aa, synthetic construct
GIIDAVYDFGKGFVDGFKAAYARDRDK

SEQ ID NO: 1554 - protein, 20 aa, synthetic construct
MGQGALNSYRDAWKYGFRAG

SEQ ID NO: 1555 - protein, 20 aa, synthetic construct
MGQGALNSYRDAWKYGLRAG

SEQ ID NO: 1556 - protein, 24 aa, synthetic construct
AEAVVNGGKAIANAGIWIWNQLVD

SEQ ID NO: 1557 - protein, 37 aa, synthetic construct
SGLTAGYSLVRRTPALPAVATGLAISVITDAILSMTI

SEQ ID NO: 1558 - protein, 37 aa, synthetic construct; identical to SEQ ID NO: 1557

SEQ ID NO: 1559 - protein, 42 aa, synthetic construct
LGRGLGCGRGCGGCTPASNPSGPSSPSAPSAPSTSSASSVLV

SEQ ID NO: 1560 - protein, 14 aa, synthetic construct
DFLKGIKAGWDSYN

SEQ ID NO: 1561 - protein, 42 aa, synthetic construct
TGYEFFAGYISDVFYDEDGNGQVNSDAEAGSYISVIADPPKP

SEQ ID NO: 1562 - protein, 29 aa, synthetic construct
ELLGGAGHGVNTISAECRWNSLQAIFSCC

SEQ ID NO: 1563 - protein, 29 aa, synthetic construct
ELLGGAGHGVNTISAECRWNSLQAIFSCC

SEQ ID NO: 1564 - protein, 29 aa, synthetic construct
ELLGGAGHGVNTISAECRWNSLQAIFSCC

SEQ ID NO: 1565 - protein, 31 aa, synthetic construct
GAGRAIYEGVRTVSPAVRIGEWIAGGRYPWQ

SEQ ID NO: 1566 - protein, 14 aa, synthetic construct
VNSWASGVASFAAY

SEQ ID NO: 1567 - protein, 13 aa, synthetic construct
LGAGLYQVGKNLK

SEQ ID NO: 1568 - protein, 42 aa, synthetic construct
GGRPGESVGESVPDCAVRDLTAGPPEMVAHQHRTEPGCAGGQ

SEQ ID NO: 1569 - protein, 41 aa, synthetic construct
GMAEGAIGGALAGGPIGAGIGIGLGQIGGSIYCIGNILGGK

SEQ ID NO: 1570 - protein, 17 aa, synthetic construct
IAGGVTAVGTAIWGAFK

SEQ ID NO: 1571 - protein, 26 aa, synthetic construct
AAANALVAAADTIASHPVGGPRGGYN

SEQ ID NO: 1572 - protein, 37 aa, synthetic construct; identical to SEQ ID NO: 1536

SEQ ID NO: 1573 - protein, 19 aa, synthetic construct
GGAGGLLELGAAAYIMAYC

SEQ ID NO: 1574 - protein, 42 aa, synthetic construct
GGLAVWEDGYSRWLYYREFAPYMRQGALNSYIDAWKYGFRTG

SEQ ID NO: 1575 - protein, 42 aa, synthetic construct; identical to SEQ ID NO: 1574

SEQ ID NO: 1576 - protein, 21 aa, synthetic construct
GIFEDAVKWCDKLFSTGRHFA

SEQ ID NO: 1577 - protein, 21 aa, synthetic construct
GIFEDAVKWCDKLFSTGRHFA

SEQ ID NO: 1578 - protein, 42 aa, synthetic construct; identical to SEQ ID NO: 1512

SEQ ID NO: 1579 - protein, 42 aa, synthetic construct; identical to SEQ ID NO: 1574

SEQ ID NO: 1580 - protein, 20 aa, synthetic construct
MGQGALNSYRDAWKYGFRAG

SEQ ID NO: 1581 - protein, 20 aa, synthetic construct
IGQGALNSYRDAWKYGFRAG

SEQ ID NO: 1582 - protein, 23 aa, synthetic construct
ISGGGAIGSGMVSGMLLAKPLNH

SEQ ID NO: 1583 - protein, 42 aa, synthetic construct; identical to SEQ ID NO: 1574

SEQ ID NO: 1584 - protein, 42 aa, synthetic construct; identical to SEQ ID NO: 1574

SEQ ID NO: 1585 - protein, 42 aa, synthetic construct; identical to SEQ ID NO: 1574

SEQ ID NO: 1586 - protein, 42 aa, synthetic construct; identical to SEQ ID NO: 1574

SEQ ID NO: 1587 - protein, 27 aa, synthetic construct
GGAEGINSFFSTAWNNDPVVKWVRSIE

SEQ ID NO: 1588 - protein, 16 aa, synthetic construct
EMIRQGIDDFKEGWNE

SEQ ID NO: 1589 - protein, 14 aa, synthetic construct
YMGWGNSGGNGAWG

SEQ ID NO: 1590 - protein, 23 aa, synthetic construct
LATDAFNHADQLIDGFKQGWERP

SEQ ID NO: 1591 - protein, 24 aa, synthetic construct
LATDAFNHADQLIDGFKQGWERPW

SEQ ID NO: 1592 - protein, 38 aa, synthetic construct
VEEYCATLTNMMDGEYAKTEWTAEQWTNAWNAYSKHCK

SEQ ID NO: 1593 - protein, 40 aa, synthetic construct; identical to SEQ ID NO: 987

SEQ ID NO: 1594 - protein, 38 aa, synthetic construct
QVYGGGFDEGKSVGYQIGKTISGAIALYGFYVLVAALV

SEQ ID NO: 1595 - protein, 12 aa, synthetic construct
AGYTIGKDIAKR

SEQ ID NO: 1596 - protein, 42 aa, synthetic construct
GKGAVTGALTGAGFGGAPGAILGAVYGAPFGALDYVISDRLK

SEQ ID NO: 1597 - protein, 42 aa, synthetic construct; identical to SEQ ID NO: 1596

SEQ ID NO: 1598 - protein, 32 aa, synthetic construct
SGGLAVWEDGYSRWLYYREFAPYMRQGHLILI

SEQ ID NO: 1599 - protein, 24 aa, synthetic construct
IGAGIVAGLGGGFFGGYALARIFG

SEQ ID NO: 1600 - protein, 24 aa, synthetic construct
IGAGIVAGLGGGFFGGYALARIFG

SEQ ID NO: 1601 - protein, 42 aa, synthetic construct
SALGTGGALMAAANGARWGAVGGVAGMAVGAAAGFAYYYYFS

SEQ ID NO: 1602 - protein, 38 aa, synthetic construct
YNKMEKILEKLSPEELDSIKGGTWVLIDTGWVWIEGTI

SEQ ID NO: 1603 - protein, 38 aa, synthetic construct
YNKMEKILEKLSPEELDSIKGGTWVLTDTGWVWIEGTI

SEQ ID NO: 1604 - protein, 24 aa, synthetic construct
SSADSALQSVNRITNALHLIYLNI

SEQ ID NO: 1605 - protein, 12 aa, synthetic construct
WEEWCGLYDKYC

SEQ ID NO: 1606 - protein, 14 aa, synthetic construct
KWCDKLFSTGRHFA

SEQ ID NO: 1607 - protein, 21 aa, synthetic construct
GIFEDAVKWCDKLFSTGRHFA

SEQ ID NO: 1608 - protein, 14 aa, synthetic construct
KGLTSGIADGWNKR

SEQ ID NO: 1609 - protein, 41 aa, synthetic construct
GVLATAAGKIAYCIGYGALNRGCDINGNPYPWDGFISATVR

SEQ ID NO: 1610 - protein, 32 aa, synthetic construct
KALQALADAYAKDGATNRQWDEWCDAYEAHGC

SEQ ID NO: 1611 - protein, 37 aa, synthetic construct
LSKVSGGASQMTHSETVKGTGGSGQGVDVVYDQSVDW

SEQ ID NO: 1612 - protein, 40 aa, synthetic construct
GGGGGGGGSFSLSDLPGPQTTPYYGDAYQYRFAGRHWGTN

SEQ ID NO: 1613 - protein, 26 aa, synthetic construct
KVAGAVVTIGGALYGAGYAIGKALRP

SEQ ID NO: 1614 - protein, 26 aa, synthetic construct
KVAGAVVTIGGALYGAGYAIGKALRP

SEQ ID NO: 1615 - protein, 37 aa, synthetic construct
GLIGDLLGTVGQIVTPLPIVGPILGGILGNIGGKFPL

SEQ ID NO: 1616 - protein, 34 aa, synthetic construct
GILSSVAGLVKDTWGTLYSTGRDFGRSVVNAAMP

SEQ ID NO: 1617 - protein, 19 aa, synthetic construct
SLGNNIGGSLEGLFKSLTS

SEQ ID NO: 1618 - protein, 12 aa, synthetic construct
IIDFGQGFLDAF

SEQ ID NO: 1619 - protein, 30 aa, synthetic construct
VQAATGIVSHVPGLVTGVTGVSVNAGRAGL

SEQ ID NO: 1620 - protein, 27 aa, synthetic construct
GGGDGVFRTISHECAMNTWMFIFTCCS

SEQ ID NO: 1621 - protein, 14 aa, synthetic construct
KWCNKLFGTGRHVA

SEQ ID NO: 1622 - protein, 14 aa, synthetic construct
KWCNKLFGTGRHVA

SEQ ID NO: 1623 - protein, 14 aa, synthetic construct
KWCNKLFGTGRHVA

SEQ ID NO: 1624 - protein, 41 aa, synthetic construct
DDMEKVAGGLLDLKCVNNNVPLCGCSQTSTEKLQQATKDQG

SEQ ID NO: 1625 - protein, 41 aa, synthetic construct
EGGTDWVKCYAGTIGSAILGFGAMGPIGYWAGAGVGYASFC

SEQ ID NO: 1626 - protein, 28 aa, synthetic construct
TIGSALVGSAGGPVGYRGGALVGYATFC

SEQ ID NO: 1627 - protein, 27 aa, synthetic construct
KVIGGVAGVIAASGAAGIAAGYYANRP

SEQ ID NO: 1628 - protein, 23 aa, synthetic construct
GVGIVAGLGGGFFGGYTLARLFG

SEQ ID NO: 1629 - protein, 27 aa, synthetic construct
KVIGGVAGVIAASGAAGIAAGYYANRP

SEQ ID NO: 1630 - protein, 27 aa, synthetic construct
KVIGGVAGVIAASGAAGIAAGYYANRP

SEQ ID NO: 1631 - protein, 42 aa, synthetic construct
CDTAYSKMTDYASQAEDPEIKKLFQDAADSAMQNRQELMKFL

SEQ ID NO: 1632 - protein, 35 aa, synthetic construct
GWVGAAIGAISGGLGYAGSKVGDKMSERTTSTTDE

SEQ ID NO: 1633 - protein, 19 aa, synthetic construct
SIGNNIGGSLKGLFDSLSR

SEQ ID NO: 1634 - protein, 20 aa, synthetic construct
MGQGALNSYRDAWKYGFRAG

SEQ ID NO: 1635 - protein, 37 aa, synthetic construct
LEEVNGGGAGAVIAVVVVICVIAFAVGVYNGYQDNKK

SEQ ID NO: 1636 - protein, 40 aa, synthetic construct
ALAASMADKIAQWGAAADRMTKGDNAASGIDISRAMAGGR

SEQ ID NO: 1637 - protein, 26 aa, synthetic construct
DYLKDLYESAGAAVAAGAAAGAAAIR

SEQ ID NO: 1638 - protein, 12 aa, synthetic construct
TTLSQFFSKLFG

SEQ ID NO: 1639 - protein, 16 aa, synthetic construct
VSAGAGIAAGYYANRP

SEQ ID NO: 1640 - protein, 34 aa, synthetic construct
AAIGSLVAPGAGTWAGGILGAQYCTAIWGIARSL

SEQ ID NO: 1641 - protein, 34 aa, synthetic construct; identical to SEQ ID NO: 1640

SEQ ID NO: 1642 - protein, 34 aa, synthetic construct; identical to SEQ ID NO: 1640

SEQ ID NO: 1643 - protein, 27 aa, synthetic construct
KIGAAVAGGAAALFAGGAALGYYANRP

SEQ ID NO: 1644 - protein, 27 aa, synthetic construct
KLIGGVAALATASAGAGIAAGYYANRP

SEQ ID NO: 1645 - protein, 27 aa, synthetic construct
KIGAAVAGGAAALFAGGAALGYYANRP

SEQ ID NO: 1646 - protein, 16 aa, synthetic construct
VSAGAGIAAGYYANRP

SEQ ID NO: 1647 - protein, 23 aa, synthetic construct
AAKIAGGITAAGIAGYNYVTGRW

SEQ ID NO: 1648 - protein, 27 aa, synthetic construct
KLIGGVAALATASAGAGIAAGYYANRP

SEQ ID NO: 1649 - protein, 16 aa, synthetic construct
VSAGAGIAAGYYANRP

SEQ ID NO: 1650 - protein, 27 aa, synthetic construct
KIGAAVAGGAAALFAGGAALGYYANRP

SEQ ID NO: 1651 - protein, 16 aa, synthetic construct
VSAGAGIAAGYYANRP

SEQ ID NO: 1652 - protein, 42 aa, synthetic construct
FLSSVGGAAEGVYYCAGVGALTPYTVVGCAGIGAVVGAFFPH

SEQ ID NO: 1653 - protein, 15 aa, synthetic construct
GCGVVGAGLGYAFPH

SEQ ID NO: 1654 - protein, 22 aa, synthetic construct
AGVSAGGGCASAGLSLIGSACH

SEQ ID NO: 1655 - protein, 19 aa, synthetic construct
VGHALSAGVAVGFAALATN

SEQ ID NO: 1656 - protein, 19 aa, synthetic construct
VGHALSAGVAVGFAALATN

SEQ ID NO: 1657 - protein, 38 aa, synthetic construct
SVLTGVVNAVANGISAGIKVINMDQKETYTGKYGSVTF

SEQ ID NO: 1658 - protein, 14 aa, synthetic construct
AMAVGGAAAGSFCR

SEQ ID NO: 1659 - protein, 14 aa, synthetic construct
AMAVGGAAAGSFCR

SEQ ID NO: 1660 - protein, 14 aa, synthetic construct
KWCDKLFSTGRHFA

SEQ ID NO: 1661 - protein, 21 aa, synthetic construct
GIFEDAVKWCDKLFSTGRHFA

SEQ ID NO: 1662 - protein, 14 aa, synthetic construct
AMAVGGAAAGSFCR

SEQ ID NO: 1663 - protein, 34 aa, synthetic construct
AGAAAFYGGASAASAIGTTIGGIATTVGTATTVF

SEQ ID NO: 1664 - protein, 21 aa, synthetic construct
GIFEDAVKWCDKLFSTGRHFA

SEQ ID NO: 1665 - protein, 23 aa, synthetic construct
SDMAGLIGGAITITNTAQETLKS

SEQ ID NO: 1666 - protein, 26 aa, synthetic construct
IIGSMGGPWGALGGGVTGAALCYLND

SEQ ID NO: 1667 - protein, 32 aa, synthetic construct
LNSVGGGYEDAYKNYFDDKNPLKPMFEDYVKS

SEQ ID NO: 1668 - protein, 30 aa, synthetic construct
KRACQGICAALAMSPGTFIALAGAAVLTKN

SEQ ID NO: 1669 - protein, 21 aa, synthetic construct
AAMAVGGSIVSGYLVGTGLRP

SEQ ID NO: 1670 - protein, 19 aa, synthetic construct
GGPAGMAAGCAVGVVESFF

SEQ ID NO: 1671 - protein, 23 aa, synthetic construct
YKKVANYLREIDPEDFILGGPQL

SEQ ID NO: 1672 - protein, 34 aa, synthetic construct
SHGGSDATSGASDASGHAGDASPAADATSGASSY

SEQ ID NO: 1673 - protein, 16 aa, synthetic construct
VAVGATIGGGVALLGC

SEQ ID NO: 1674 - protein, 31 aa, synthetic construct
LEEINGGIAGLIIGCIGIAAICYYFGDHSAR

SEQ ID NO: 1675 - protein, 16 aa, synthetic construct
VAVGATIGGGVALLGC

SEQ ID NO: 1676 - protein, 12 aa, synthetic construct
EGLQAGWEAAGK

SEQ ID NO: 1677 - protein, 25 aa, synthetic construct
SAYQAGYATGRAIAIAAVAATFAAL

SEQ ID NO: 1678 - protein, 31 aa, synthetic construct
EYISGGFNLFGAAAASPALSPTLASASPLLF

SEQ ID NO: 1679 - protein, 27 aa, synthetic construct
KVGAAVAGGATALFAGGAALGYYANRP

SEQ ID NO: 1680 - protein, 39 aa, synthetic construct
EELMNVGGGALEAAPASTGSAGGKVTSGGDWEVNVGIDW

SEQ ID NO: 1681 - protein, 16 aa, synthetic construct
VAVGATIGGGVALLGC

SEQ ID NO: 1682 - protein, 34 aa, synthetic construct
EELEGVAGGAIGQQSGSMGDLWFACYIGEACKGE

SEQ ID NO: 1683 - protein, 22 aa, synthetic construct
SGGNNFGDLLRQWWWSVFRGDD

SEQ ID NO: 1684 - protein, 27 aa, synthetic construct
KLIGGVAALATTSAGVGVAAGYYANRP

SEQ ID NO: 1685 - protein, 27 aa, synthetic construct
KVGAAVVGGATALFAGGAALGYYANRP

SEQ ID NO: 1686 - protein, 27 aa, synthetic construct
KVGAAVVGGATALFAGGATLGYYANRP

SEQ ID NO: 1687 - protein, 23 aa, synthetic construct
AAKIAGGITAVGIAGYNYVTGRW

SEQ ID NO: 1688 - protein, 27 aa, synthetic construct
KIGAAVAGGAAALFAGGAALGYYANRP

SEQ ID NO: 1689 - protein, 23 aa, synthetic construct
AAVAGGAALFAGGAALGYYANRP

SEQ ID NO: 1690 - protein, 27 aa, synthetic construct
KIGAAVAGGAAALFAGGAALGYYANRP

SEQ ID NO: 1691 - protein, 27 aa, synthetic construct
KLIGGVAALATASAGAGIAAGYYANRP

SEQ ID NO: 1692 - protein, 27 aa, synthetic construct
KIGAAVAGGAAALFAGGAALGYYANRP

SEQ ID NO: 1693 - protein, 27 aa, synthetic construct
KVGGAVVGGATALFAGGAALGYYANRP

SEQ ID NO: 1694 - protein, 19 aa, synthetic construct
GGGIFEDIYHWLHLLPEPR

SEQ ID NO: 1695 - protein, 19 aa, synthetic construct
GGGIFEDIYHWLHLLPEPR

SEQ ID NO: 1696 - protein, 32 aa, synthetic construct
SQGESAAGDGYAIKCCNNKGKQEQQKTVDPTT

SEQ ID NO: 1697 - protein, 19 aa, synthetic construct
GGGIFEDIYHWLHLLPEPR

SEQ ID NO: 1698 - protein, 19 aa, synthetic construct
GGGIFEDIYHWLHLLPEPR

SEQ ID NO: 1699 - protein, 20 aa, synthetic construct
GGGIYDKINKFLSNSKHYFI

SEQ ID NO: 1700 - protein, 35 aa, synthetic construct
GQHVGSILTTADGDPVAVYVDGVLQNSVTDGFYHG

SEQ ID NO: 1701 - protein, 32 aa, synthetic construct; identical to SEQ ID NO: 1696

SEQ ID NO: 1702 - protein, 32 aa, synthetic construct; identical to SEQ ID NO: 1696

SEQ ID NO: 1703 - protein, 32 aa, synthetic construct; identical to SEQ ID NO: 1696

SEQ ID NO: 1704 - protein, 22 aa, synthetic construct
AGEVCTLAGAMIGSLVAPRAGT

SEQ ID NO: 1705 - protein, 22 aa, synthetic construct
AGEVCTLAGAMIGSLVAPRSGT

SEQ ID NO: 1706 - protein, 33 aa, synthetic construct
NGGGIWTEVFKALIASYEMASDFKEGWDSVPDL

SEQ ID NO: 1707 - protein, 42 aa, synthetic construct
GGGSHGGGYYSSVGGGSYGGGYCGGGCGGYGYGFGFWGGSFS

SEQ ID NO: 1708 - protein, 42 aa, synthetic construct; identical to SEQ ID NO: 1707

SEQ ID NO: 1709 - protein, 42 aa, synthetic construct; identical to SEQ ID NO: 1707

SEQ ID NO: 1710 - protein, 19 aa, synthetic construct
GIYQVGEAAGKTFYYITHP

SEQ ID NO: 1711 - protein, 19 aa, synthetic construct
GIYQVGEAAGKTFYYITHP

SEQ ID NO: 1712 - protein, 19 aa, synthetic construct
GIYQAGEAAGKTFYYITHP

SEQ ID NO: 1713 - protein, 19 aa, synthetic construct
GIYQVGEAAGKTFYYITHP

SEQ ID NO: 1714 - protein, 19 aa, synthetic construct
GGGIFEDIYHWLHLLPDPR

SEQ ID NO: 1715 - protein, 19 aa, synthetic construct
GIYQVGEAAGKTFYYITHP

SEQ ID NO: 1716 - protein, 19 aa, synthetic construct
GIYQAGEAAGKTFYYITHP

SEQ ID NO: 1717 - protein, 37 aa, synthetic construct
VGVADAIVRAVNIVYETARPTLQSLPLLGPILADDEQ

SEQ ID NO: 1718 - protein, 40 aa, synthetic construct
GASEGFSAAAGGTAFFIGPYAIGTGAVGAAIGGGVALLGC

SEQ ID NO: 1719 - protein, 37 aa, synthetic construct
VAATALAPATAVGAGVILAGVAAVSAFDAFQSYQMAQ

SEQ ID NO: 1720 - protein, 40 aa, synthetic construct; identical to SEQ ID NO: 1718

SEQ ID NO: 1721 - protein, 40 aa, synthetic construct; identical to SEQ ID NO: 1718

SEQ ID NO: 1722 - protein, 37 aa, synthetic construct; identical to SEQ ID NO: 1719

SEQ ID NO: 1723 - protein, 37 aa, synthetic construct
AAATALAPVTAVGAGVILVGVAGASAIDAFQTYQMAQ

SEQ ID NO: 1724 - protein, 40 aa, synthetic construct; identical to SEQ ID NO: 1718

SEQ ID NO: 1725 - protein, 40 aa, synthetic construct; identical to SEQ ID NO: 1718

SEQ ID NO: 1726 - protein, 31 aa, synthetic construct
LGAGFAVMAAGIVGTSLAGAAMIVSICTPVL

SEQ ID NO: 1727 - protein, 31 aa, synthetic construct
LGAGFAVMAAGIVGTSLAGAAMIVSICTPIL

SEQ ID NO: 1728 - protein, 29 aa, synthetic construct
EAVYEFGSGFAENIGPAFERAFNNIKYQN

SEQ ID NO: 1729 - protein, 30 aa, synthetic construct
VLKSMNAVASAFQFIALDLNDVSTTRAVEN

SEQ ID NO: 1730 - protein, 30 aa, synthetic construct
VQAATGIVSHVPGLVTGITGVSVNAGRAGL

SEQ ID NO: 1731 - protein, 42 aa, synthetic construct
EQLEEVAGGGSHKQIEEHFEEDQNAAVPGNTTIDRGPEPQVI

SEQ ID NO: 1732 - protein, 31 aa, synthetic construct
VGDVAGTAQALAAETPASQLAQLVTVHTFGI

SEQ ID NO: 1733 - protein, 19 aa, synthetic construct
GIYQVGEAAGKTFYYITHP

SEQ ID NO: 1734 - protein, 21 aa, synthetic construct
AGGNVVTAFATEDSIALNIDK

SEQ ID NO: 1735 - protein, 34 aa, synthetic construct
AVCAVGGAVLGGPAGFLGGAFVGAILATIVASQE

SEQ ID NO: 1736 - protein, 13 aa, synthetic construct
WDGWNKAFDNACL

SEQ ID NO: 1737 - protein, 21 aa, synthetic construct
AGLAAAGAVLAGGVAVYEAWH

SEQ ID NO: 1738 - protein, 21 aa, synthetic construct
AGLAAAGAVLAGGVAVYEAWH

SEQ ID NO: 1739 - protein, 28 aa, synthetic construct
AGEVCTLAGAMIGSLVAPRAGTWAGGVL

SEQ ID NO: 1740 - protein, 42 aa, synthetic construct
FLSSVGGAAEGVYYCAGVGALTPYTVVGCAGIGAVVGSFFPQ

SEQ ID NO: 1741 - protein, 23 aa, synthetic construct
AAKIAGGITAAGIAGYNYVTGRW

SEQ ID NO: 1742 - protein, 23 aa, synthetic construct
AAKIAGGITAAGIAGYNYVTSRW

SEQ ID NO: 1743 - protein, 23 aa, synthetic construct
AAKIAGGITAAGIAGYNYVTGRW

SEQ ID NO: 1744 - protein, 23 aa, synthetic construct
AAKIAGGITAAGIAGYNYVTSRW

SEQ ID NO: 1745 - protein, 23 aa, synthetic construct
AAKIAGGITAAGIAGYNYVTSRW

SEQ ID NO: 1746 - protein, 23 aa, synthetic construct
AAKIAGGITAAGVAGYNYVTGRW

SEQ ID NO: 1747 - protein, 23 aa, synthetic construct
AAKIAGGITAAGIAGYNYVTSRW

SEQ ID NO: 1748 - protein, 23 aa, synthetic construct
AAKIAGGITAAGIAGYNYVTGRW

SEQ ID NO: 1749 - protein, 23 aa, synthetic construct
AAKIAGGITAAGIAGYNYVTSRW

SEQ ID NO: 1750 - protein, 28 aa, synthetic construct
AGEVCTLAGAMIGSLVAPRAGTWAGGVL

SEQ ID NO: 1751 - protein, 23 aa, synthetic construct
AAKIAGGITAAGIAGYNYVTSRW

SEQ ID NO: 1752 - protein, 23 aa, synthetic construct
AAKIAGGITAAGIAGYNYVTSRW

SEQ ID NO: 1753 - protein, 23 aa, synthetic construct
AYADELTRLAAALDVDIKTLLLQ

SEQ ID NO: 1754 - protein, 19 aa, synthetic construct
EAADALLAVLDEIKRRAAA

SEQ ID NO: 1755 - protein, 18 aa, synthetic construct
AMVKDGWKLIETASENAY

SEQ ID NO: 1756 - protein, 19 aa, synthetic construct
SLGNNIGGSLAGIFDSLKS

SEQ ID NO: 1757 - protein, 18 aa, synthetic construct
DFGNGVVDGFKDGLNAWK

SEQ ID NO: 1758 - protein, 26 aa, synthetic construct
TIVKSIVTLAETAMDYQASLSTSDKK

SEQ ID NO: 1759 - protein, 19 aa, synthetic construct
SLGNNIGGSLEGLFQSLTS

SEQ ID NO: 1760 - protein, 22 aa, synthetic construct
AVGAAIGYGASAAIEGIEGWFE

SEQ ID NO: 1761 - protein, 19 aa, synthetic construct
GIYQVGEAAGKTFYYITHP

SEQ ID NO: 1762 - protein, 19 aa, synthetic construct
GIYQVGEAAGKTFYYITHP

SEQ ID NO: 1763 - protein, 19 aa, synthetic construct
GIYQAGEAAGKTFYYITHP

SEQ ID NO: 1764 - protein, 19 aa, synthetic construct
GIYQAGEAAGKTFYYITHP

SEQ ID NO: 1765 - protein, 31 aa, synthetic construct
VGDVVGTVQSVAAVVPVAAATGLVSVQTTGI

SEQ ID NO: 1766 - protein, 13 aa, synthetic construct
NIDSFGQKFIQSF

SEQ ID NO: 1767 - protein, 24 aa, synthetic construct
ALLGSVGGPWGSAAGGAVGAGFFC

SEQ ID NO: 1768 - protein, 28 aa, synthetic construct
TIGSALVGSAGGPVGYWGGALVGYATFC

SEQ ID NO: 1769 - protein, 40 aa, synthetic construct
NIGSAIGGCLGGMLIAAAGGPITGGAAAFVCVASGIAAYQ

SEQ ID NO: 1770 - protein, 28 aa, synthetic construct
TIGSALVGSAGGPVGYWGGALVGYATFC

SEQ ID NO: 1771 - protein, 40 aa, synthetic construct; identical to SEQ ID NO: 1769

SEQ ID NO: 1772 - protein, 28 aa, synthetic construct
TIGSALVGSAGGPVGYWGGALVGYATFC

SEQ ID NO: 1773 - protein, 16 aa, synthetic construct
GGAGGAVAYGATCWWS

SEQ ID NO: 1774 - protein, 40 aa, synthetic construct; identical to SEQ ID NO: 1769

SEQ ID NO: 1775 - protein, 28 aa, synthetic construct
TIGSALVGSAGGPVGYWGGALVGYATFC

SEQ ID NO: 1776 - protein, 40 aa, synthetic construct; identical to SEQ ID NO: 1769

SEQ ID NO: 1777 - protein, 28 aa, synthetic construct
TIGSALVGSAGGPVGYWGGALVGYATFC

SEQ ID NO: 1778 - protein, 40 aa, synthetic construct; identical to SEQ ID NO: 1769

SEQ ID NO: 1779 - protein, 40 aa, synthetic construct; identical to SEQ ID NO: 1769

SEQ ID NO: 1780 - protein, 28 aa, synthetic construct
TIGSALVGSAGGPVGYWGGALVGYATFC

SEQ ID NO: 1781 - protein, 40 aa, synthetic construct; identical to SEQ ID NO: 1769

SEQ ID NO: 1782 - protein, 28 aa, synthetic construct
TIGSALVGSAGGPVGYWGGALVGYATFC

SEQ ID NO: 1783 - protein, 33 aa, synthetic construct
AAAAIYAGCAAGGFVAGVGVTVGTVKFLNWIGA

SEQ ID NO: 1784 - protein, 37 aa, synthetic construct
GFLKDLVNGVADFTPAGPVNRTMQRAGGPTIGDYVVG

SEQ ID NO: 1785 - protein, 13 aa, synthetic construct
WDGWNEAFDNACL

SEQ ID NO: 1786 - protein, 23 aa, synthetic construct
KAIAGGAALIGSGLAAGYFLGGD

SEQ ID NO: 1787 - protein, 35 aa, synthetic construct
LESVAGGITDLTTEASCMCCAAGATNQTPGFEEQR

SEQ ID NO: 1788 - protein, 13 aa, synthetic construct
NIDSFGEKFVQSF

SEQ ID NO: 1789 - protein, 39 aa, synthetic construct
DFVESCGKVVGRGLEAVVDEANESAREDFESGTWSSRSM

SEQ ID NO: 1790 - protein, 21 aa, synthetic construct
SGGGGGYDVVENYTNNFLKRG

SEQ ID NO: 1791 - protein, 42 aa, synthetic construct
ASYLAMGTAALGGLACTTPVGAVLYLGAEVCAGAAVIYYGAN

SEQ ID NO: 1792 - protein, 19 aa, synthetic construct
GIYQVGEAAGKTFYYITHP

SEQ ID NO: 1793 - protein, 23 aa, synthetic construct
VGSAVAGGLAGYGLYKALDWALN

SEQ ID NO: 1794 - protein, 19 aa, synthetic construct
GIYQVGEAAGKTFYYITHP

SEQ ID NO: 1795 - protein, 19 aa, synthetic construct
GIYQVGEAAGKTFYYITHP

SEQ ID NO: 1796 - protein, 40 aa, synthetic construct
NIGSAIGECLGGMLIAAAGGPITGGAAAFVCVASGIAAYQ

SEQ ID NO: 1797 - protein, 37 aa, synthetic construct; identical to SEQ ID NO: 1723

SEQ ID NO: 1798 - protein, 41 aa, synthetic construct
GAGTGGLGGAAAGSAVPVIGTGIGGAIGGVSGGLTGAATFC

SEQ ID NO: 1799 - protein, 37 aa, synthetic construct
GAVSGVVGQAGSLVSGLVGSVLGAVGGLVGGVTGALR

SEQ ID NO: 1800 - protein, 38 aa, synthetic construct
AEQMQTVIGGGGGWSIIWEDPFDPRANDPTPPPPPGNG

SEQ ID NO: 1801 - protein, 30 aa, synthetic construct
SLLAAMINDAQNNPDDFNAGVDAAPNPYNL

SEQ ID NO: 1802 - protein, 34 aa, synthetic construct
MDTVAGGAQALGFTDGNCLATITADCTPWIGICS

SEQ ID NO: 1803 - protein, 34 aa, synthetic construct; identical to SEQ ID NO: 1802

SEQ ID NO: 1804 - protein, 20 aa, synthetic construct
GIGAALIGAGGAIIASDDSI

SEQ ID NO: 1805 - protein, 20 aa, synthetic construct
GAGAGYGIAAGVDAIEGWFD

SEQ ID NO: 1806 - protein, 42 aa, synthetic construct; identical to SEQ ID NO: 1791

SEQ ID NO: 1807 - protein, 34 aa, synthetic construct
AGAVGAASGALGALPGAFTTLESDVTGLEGSLLN

SEQ ID NO: 1808 - protein, 34 aa, synthetic construct
GGVMESMNDVLKWLNGHSGSVTINNDSVYEIDFG

SEQ ID NO: 1809 - protein, 34 aa, synthetic construct
MDTVAGGAQALGFTDGNCLATITADCTDWLPICG

SEQ ID NO: 1810 - protein, 31 aa, synthetic construct
IGDVAGTAQSLAPQLPVSQFAGLVTVQSTDV

SEQ ID NO: 1811 - protein, 37 aa, synthetic construct
LELVAGGCSSWTWSLLSGLICLGYQTAKHLDNCSCPG

SEQ ID NO: 1812 - protein, 37 aa, synthetic construct
NQLSSLFATGLAGIISGTQASEQQVNGFSFKDVNAGN

SEQ ID NO: 1813 - protein, 41 aa, synthetic construct
EQLESIFGGVKAEQPGLLLAYYWEYSEGGLFASPVADYGQS

SEQ ID NO: 1814 - protein, 38 aa, synthetic construct
GGGFWKDVVNVAVGFTPLGPINRLSGALGGPTAGDLVE

SEQ ID NO: 1815 - protein, 23 aa, synthetic construct
AYADELVQLAAALDVDVETLLLQ

SEQ ID NO: 1816 - protein, 38 aa, synthetic construct
SYGGSYGGSYGGSNSSYYGGSYSGGGYYYAAVGYGYSC

SEQ ID NO: 1817 - protein, 38 aa, synthetic construct; identical to SEQ ID NO: 1816

SEQ ID NO: 1818 - protein, 16 aa, synthetic construct
AGASAIDAFQTYQMAQ

SEQ ID NO: 1819 - protein, 20 aa, synthetic construct
GVIATGAALGAAWEYFTDDE

SEQ ID NO: 1820 - protein, 34 aa, synthetic construct
YNEAINVGNQALKETDIFVAVSTQNLQTVFGGDI

SEQ ID NO: 1821 - protein, 13 aa, synthetic construct
NIDSFGEKFIQSF

SEQ ID NO: 1822 - protein, 35 aa, synthetic construct
AGAATVGGLIGGPAGAFVGGVYGGVAYAIGVAADM

SEQ ID NO: 1823 - protein, 29 aa, synthetic construct
GLGAAAGQAAGQIICPITNNGNSSEDGDG

SEQ ID NO: 1824 - protein, 36 aa, synthetic construct
GAGTVLGGLAGAGLGPGGIAAGISAGNMIAGMACSM

SEQ ID NO: 1825 - protein, 22 aa, synthetic construct
KAIAGGTALVGSGLTAGYFLGG

SEQ ID NO: 1826 - protein, 22 aa, synthetic construct
KAIAGGTALVGSGLAAGYFLGG

SEQ ID NO: 1827 - protein, 22 aa, synthetic construct
KAIAGGTALVGSGLAAGYFLGG

SEQ ID NO: 1828 - protein, 34 aa, synthetic construct
NDALNALDGLVNNPNLNTTAGNSNQQPILFNPYA

SEQ ID NO: 1829 - protein, 19 aa, synthetic construct
GVWAAATTGGAALIQSAGY

SEQ ID NO: 1830 - protein, 23 aa, synthetic construct
KVIAGGTALIGSGLAAGYFLGGD

SEQ ID NO: 1831 - protein, 13 aa, synthetic construct
QVGSFARGVWDAF

SEQ ID NO: 1832 - protein, 22 aa, synthetic construct
KAIAGGTALVGSGLAAGYFLGG

SEQ ID NO: 1833 - protein, 29 aa, synthetic construct
ETLETVVGGYSSDDGSGEPSEGPSCNTLC

SEQ ID NO: 1834 - protein, 16 aa, synthetic construct
STICTRMTGYGNGIIP

SEQ ID NO: 1835 - protein, 19 aa, synthetic construct
AGIAALGTGIATTGSLVTD

SEQ ID NO: 1836 - protein, 12 aa, synthetic construct
AVGAGAGALSDY

SEQ ID NO: 1837 - protein, 33 aa, synthetic construct
MDTVAGGAQALGFTDGNCLATITADCTPWIWGC

SEQ ID NO: 1838 - protein, 16 aa, synthetic construct
STICTRMTGYGNGIIP

SEQ ID NO: 1839 - protein, 29 aa, synthetic construct
GIGAAAGQAAGQIICPMTNNGNSSEDGDG

SEQ ID NO: 1840 - protein, 23 aa, synthetic construct
KAIAGGTALIGSGLAAGYFLGGD

SEQ ID NO: 1841 - protein, 23 aa, synthetic construct
KAIAGGTALIGSGLAAGYFLGGD

SEQ ID NO: 1842 - protein, 23 aa, synthetic construct
KAIAGGTALIGSGLAAGYFLGGD

SEQ ID NO: 1843 - protein, 23 aa, synthetic construct
KAIAGGTALVGSGLAAGYFLGGD

SEQ ID NO: 1844 - protein, 23 aa, synthetic construct
KAIAGGTALIGSGLAAGYFLGGD

SEQ ID NO: 1845 - protein, 23 aa, synthetic construct
KAIAGGTALIGSGLAAGYFLGGD

SEQ ID NO: 1846 - protein, 23 aa, synthetic construct
KAIAGGTALIGSGLAAGYFLGGD

SEQ ID NO: 1847 - protein, 23 aa, synthetic construct
KAIAGGTALVGSGLAAGYFLGGD

SEQ ID NO: 1848 - protein, 23 aa, synthetic construct
KAIAGGTALIGSGLAAGYFLGGD

SEQ ID NO: 1849 - protein, 23 aa, synthetic construct
KAIAGGTALIGSGLAAGYFLGGD

SEQ ID NO: 1850 - protein, 23 aa, synthetic construct
KAIAGGTALIGSGLAAGYFLGGD

SEQ ID NO: 1851 - protein, 23 aa, synthetic construct
KAIAGGTALIGSGLAAGYFLGGD

SEQ ID NO: 1852 - protein, 23 aa, synthetic construct
KAIAAGAALVGSGLAAGYFLGGD

SEQ ID NO: 1853 - protein, 23 aa, synthetic construct
KAIAGGTALIGSGLAAGYFLGGD

SEQ ID NO: 1854 - protein, 23 aa, synthetic construct
KVIAGGTALIGSGLAAGYFLGGD

SEQ ID NO: 1855 - protein, 23 aa, synthetic construct
KAIAGGTALIGSGWAAGYFLGGD

SEQ ID NO: 1856 - protein, 23 aa, synthetic construct
KAIAGGTALIGSGLAAGYFLGGD

SEQ ID NO: 1857 - protein, 23 aa, synthetic construct
KAIAGGTALIGSGLAAGYFLGGD

SEQ ID NO: 1858 - protein, 23 aa, synthetic construct
KAIAGGTALIGSGLAAGYFLGGD

SEQ ID NO: 1859 - protein, 23 aa, synthetic construct
KAIAGGTALIGSGLAAGYFLGGD

SEQ ID NO: 1860 - protein, 23 aa, synthetic construct
KAIAGGTALIGSGLAAGYFLGGD

SEQ ID NO: 1861 - protein, 23 aa, synthetic construct
KAIAGGTALIGSGLAAGYFLGGD

SEQ ID NO: 1862 - protein, 23 aa, synthetic construct
KAIAGGTALIGSGLAAGYFLGGD

SEQ ID NO: 1863 - protein, 23 aa, synthetic construct
KAIAGGTALIGSGLAAGYFLGGD

SEQ ID NO: 1864 - protein, 23 aa, synthetic construct
KAIAGGTALIGSGLAAGYFLGGD

SEQ ID NO: 1865 - protein, 23 aa, synthetic construct
KVIAGGTALIGSGLAAGYFLGGD

SEQ ID NO: 1866 - protein, 23 aa, synthetic construct
KAIAGGTALIGSGLAAGYFLGGD

SEQ ID NO: 1867 - protein, 14 aa, synthetic construct
FSSVSNAVAEVAAE

SEQ ID NO: 1868 - protein, 23 aa, synthetic construct
KAIAGGTALVGSGLAAGYFLGGD

SEQ ID NO: 1869 - protein, 21 aa, synthetic construct
AGGVLGGAGGTVAYGATCWWS

SEQ ID NO: 1870 - protein, 21 aa, synthetic construct
AGGVLGGAGGTVAYGATCWWS

SEQ ID NO: 1871 - protein, 35 aa, synthetic construct
GCAATGVGIGGAAGSVPGAVIGGAVGTIVGIMIAF

SEQ ID NO: 1872 - protein, 22 aa, synthetic construct
GVGAVAGAALAGAVVGVTALVG

SEQ ID NO: 1873 - protein, 25 aa, synthetic construct
GGLGAVGGPWGAVGGVLVGAALYCF

SEQ ID NO: 1874 - protein, 25 aa, synthetic construct
GGLGAVGGPWGAVGGVLVGAALYCF

SEQ ID NO: 1875 - protein, 25 aa, synthetic construct
GGLGAVGGPWGAVGGVLVGAALYCF

SEQ ID NO: 1876 - protein, 25 aa, synthetic construct
GGLGAVGGPWGAVGGVLVGAALYCF

SEQ ID NO: 1877 - protein, 25 aa, synthetic construct
AAQVAGGAGAVFLAGMGIGAAVAAQ

SEQ ID NO: 1878 - protein, 36 aa, synthetic construct
ALVGAGVALAAGAGVALAGAGALAAAILLPLYQNPD

SEQ ID NO: 1879 - protein, 14 aa, synthetic construct
GAVTGLASGLTAGL

SEQ ID NO: 1880 - protein, 14 aa, synthetic construct
GAVTGLASGLTAGL

SEQ ID NO: 1881 - protein, 34 aa, synthetic construct
VSGAVGTATGLVDGATGVNTSGVVGTATGLVSGL

SEQ ID NO: 1882 - protein, 28 aa, synthetic construct
GLVDTAVGTVEGVTGLNTAPVTNLVAGL

SEQ ID NO: 1883 - protein, 20 aa, synthetic construct
GAIATGAALGAAWEYFTDDE

SEQ ID NO: 1884 - protein, 20 aa, synthetic construct
GAIATGAALGAAWEYFTDDE